(19) United States
(12) Patent Application Publication
(10) Pub. No.: US 2025/0006370 A1
(43) Pub. Date: Jan. 2, 2025

(54) Systems and Methods for Analysis of Patient-Reported Outcome Data

(71) Applicant: AMPEL BioSolutions, LLC, Charlottesville, VA (US)

(72) Inventors: Peter E. LIPSKY, Charlottesville, VA (US); Amrie C. GRAMMER, Charlottesville, VA (US); Kate OWEN, Charlottesville, VA (US); Kristy BELL, Charlottesville, VA (US); John M. ZENT, Charlottesville, VA (US)

(21) Appl. No.: 18/709,696

(22) PCT Filed: Oct. 11, 2022
(86) PCT No.: PCT/US2022/046337

§ 371 Date: May 13, 2024

Related U.S. Application Data
(60) Provisional application No. 63/254,775, filed on Oct. 12, 2021.

(51) Int. Cl. G16H 50/20; G16H 10/60; G16H 20/10; G16H 80/00

(52) U.S. Cl. CPC G16H 50/20; G16H 10/60; G16H 20/10; G16H 80/00

(57) ABSTRACT

Disclosed are computer-implemented methods, systems, and media for analyzing patient-reported outcome (PRO) data. A method for determining a method of evaluation and treatment for patients may include receiving PRO data; validating the PRO data; inputting the PRO data to a first machine learning model; generating, using the first machine learning model, based on the PRO data. i) a score indicative of at least one of an activity of the patient's disease, an effectiveness of current treatment, or severity of the patient's reactions, and/or ii) an inference indicative of the disease state of the patient; and generating, recommending, and/or selecting based on the score and/or the inference, an action item, a second method of evaluation, and/or a second method of treatment for the patient.

This application claims priority to U.S. Provisional Patent Application No. 63/254,775, filed Oct. 12, 2021, which is incorporated in full herein by reference.

### BACKGROUND OF THE INVENTION

Patient-reported outcomes (PROs) represent the impact of a disease or condition on an individual patient without interpretation by a Health Care Professional (HCP). Although documenting the patient's experience with an illness, this information does not always correlate with that collected by the HCP and is not always integrated into the health care plan. Some data analysis may lack repeated collection of PRO information in the patient's environment with a validated mobile device and comprehensive analysis of the resultant information, may lack an ability to inform HCPs of changes in the health status of an individual, and may lack an ability provide useful information to fully evaluate patient status more accurately and make therapeutic decisions more effectively.

### SUMMARY OF THE INVENTION

In some embodiments, mobile device technology may be used to collect PRO data in an electronic format (ePRO). In particular, applications executed by mobile devices may present questionnaires with questions regarding a patient's health. The applications may receive patient inputs that indicate the patient's perception of their own health. In addition, mobile devices may collect biometric data (e.g., sleep data, breathing data, body temperature data, heart rate data, etc.), device motion data (e.g., indicative of activity, steps, etc.), and/or geo-location data from patients. The PRO and/or other data may be used assess a patient's health. In particular, the PRO and/or other data may be used to generate a score representative of a health care professional's (HCP's) evaluation of the patient. For example, the PRO and/or other data may be used to generate a physician's assessment score in an automated fashion, such as by using machine learning that inputs the PRO and/or biometric data, including ePRO and paper-administered PRO data, and generates a score that estimates what a physician would generate if a physician were analyzing the patient's data. For example, to determine agreement between PRO information collected in different formats, intra-class correlation coefficients (ICCs), paired t-tests, and Band-Altman plots may be evaluated, along with compliance and Cronbach's alpha as a measure of survey reliability.

In some embodiments, a method for determining a method of evaluation and treatment of patients may include receiving, by a first device, PRO data from at least one other device, the PRO data including ePRO data and/or paper-administered data, indicating patient reactions to their health, and/or patient reactions to a method of treatment using a pharmaceutical or biological treatment. The first device may validate the PRO data according to regulatory requirements. The first device may input the PRO data, along with any collected device and/or biometric data received from at least one other device, to a machine learning model. The machine learning model may include one or multiple layers that analyze the input data, and that may be trained to generate health scores indicative of what a healthcare professional would generate to assess a patient's health and/or the effectiveness of a current healthcare treatment and/or severity a patient's reactions to a treatment. The machine learning model may generate the score based on the inputs, and if trained, based on training data. A second machine learning model or a layer of the machine learning model may generate, based on the score, a method of evaluation and/or treatment for a patient. The method of evaluation and/or treatment for the patient as generated by the machine learning model or a layer of the machine learning model may be a different method of evaluation and/or treatment for the patient than the method of evaluation and/or treatment for the patient being evaluated using the PRO data (e.g., if the score indicates a low effectiveness or high activity of the patient's disease/condition), or may be the same method of evaluation and/or treatment for the patient as the method of evaluation and/or treatment for the patient being evaluated using the PRO data (e.g., when the score indicates a high effectiveness of the current method of evaluation and/or treatment or a low activity of the patient's disease/condition).

In some embodiments, a method for predicting the Physician's Global Assessment Score may include receiving, by a first device, PRO data from at least one other device, the PRO data including ePRO data and/or paper-administered data, indicating patient reactions to their health, and/or patient reactions to a method of treatment using a pharmaceutical or biological treatment. The first device may validate the PRO data according to regulatory requirements. The first device may receive documents with patient data from patient visits to a doctor. The first device may input the PRO data and the documents to a first machine learning model, which may generate, based on the PRO data and the documents, a prediction of a Physician's Global Assessment Score indicative of a patient's status or the severity of the patient's reactions. A second machine learning model or layer of the first machine learning model may generate, a prediction of the Physician's Global Assessment Score and a second method of treatment for the patient. The method of evaluation and/or treatment for the patient as generated by the machine learning model or a layer of the machine learning model may be a different method of evaluation and/or treatment for the patient than the method of evaluation and/or treatment for the patient being evaluated using the PRO data (e.g., if the Physician's Global Assessment Score indicates a low effectiveness or high activity of the patient's disease/condition), or may be the same method of evaluation and/or treatment for the patient as the method of evaluation and/or treatment for the patient being evaluated using the PRO data (e.g., when the Physician's Global Assessment Score indicates a high effectiveness of the current method of evaluation and/or treatment or a low activity of the patient's disease/condition).

In some embodiments, a method of treatment for patients having Lupus may include receiving, by a first device, PRO data from a second device, the PRO data indicative of a patient's reactions to a method of treatment for Lupus using a pharmaceutical or biological treatment. The first device may validate the patient reported outcome data according to regulatory requirements. The first device may receive documents with data from patient visits to a doctor. The first device may input the patient reported outcome data and the documents to a first machine learning model, which may generate, based on the PRO data and the documents, a prediction of the Systematic Lupus Erythematosus Disease Activity Index (SLEDAI) Score indicative of activity of the patient's disease and reactions. A second machine learning model or layer of the first machine learning model may generate, based on the prediction of the SLEDAI Score, a method of evaluation and/or treatment for the patient. The method of evaluation and/or treatment for the patient as generated by the machine learning model or a layer of the machine learning model may be a different method of evaluation and/or treatment for the patient than the method of evaluation and/or treatment for the patient being evaluated using the PRO data (e.g., if the SLEDAI Score indicates a low effectiveness or high activity of the patient's disease/condition), or may be the same method of evaluation and/or treatment for the patient as the method of evaluation and/or treatment for the patient being evaluated using the PRO data (e.g., when the SLEDAI Score indicates a high effectiveness of the current method of evaluation and/or treatment or a low activity of the patient's disease/condition).

In some embodiments, to validate PRO data, a device may verify the equivalence of paper and electronic administration methods for patient surveys. ICC values may be used to verify that the equivalence of paper and electronic administration methods for patient surveys improves as more surveys are taken and provided. Bland-Altman plots may be used to identify any bias between paper and ePRO data. To account for any discrepancies between paper and ePRO data, a machine learning model may adjust weights used to evaluate the paper and ePRO data. For example, a higher weight may be used for data with higher reliability.

In some embodiments, a machine learning model used to generate predicted health scores may be trained to generate the scores so that no adjustment is needed to a current method of treatment a certain percentage of the time (e.g., 80% or some other number). For example, when the health score indicates that a method of treatment should be changed (e.g., the score is above a threshold that indicates severity of a condition or patient's reactions), the machine learning model may adjust the method of treatment. Therefore, the machine learning model may be trained to generate a score that is below the threshold value a certain percentage of the time. For example, if a Physician's Global Assessment Score indicates that 80% of the time the physician would not change the patient's current method of treatment, the training data may train the machine learning model to generate a score that would satisfy the threshold 80% of the time. In this manner, the input data to the machine learning model would result in a score that does not satisfy the threshold 20% of the time. To achieve the adjustment, the machine learning model may adjust the weight of certain input data up or down. For example, to increase the rate at which the score results in a change in treatment, the weights of certain data may be decreased to produce a lower score (or increased to produce a higher score, depending on the thresholds used). In this manner, one patient's data may result in a change in treatment, while another patient's data may not result in a change in treatment, based on the evaluation criteria used by the machine learning model to generate and evaluate a score.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a system for analyzing patient-reported outcome (PRO) data according to embodiments described herein;

FIG. 2 shows a non-limiting example of application-based PRO data collection using the systems and methods described herein;

FIG. 3 shows a non-limiting example of PRO data compared to physician measures using the systems and methods described herein;

FIGS. 4A and B show non-limiting examples of work flow for analyzing PRO data according to embodiments described herein;

FIG. 5 shows a non-limiting example of a machine learning work flow for analyzing PRO data according to embodiments described herein;

FIGS. 6A-H show non-limiting examples of unsupervised clustering for analyzing PRO data according to embodiments described herein;

FIG. 7 shows non-limiting examples of curves for analyzing PRO data according to embodiments described herein;

FIG. 8 shows a block diagram illustrating an example of a computing device or computer system upon which any of one or more techniques (e.g., methods) may be performed according to embodiments described herein.

FIGS. 9A-C show patient compliance with application (app)-based electronic patient-reported outcome (ePRO) completion. FIG. 9A: Boxplots showing the compliance summary according to the survey schedule. Each point represents the compliance of one of the 62 patients. Asterisks (*) indicate a significant (P<0.05) difference between surveys according to a Wilcoxon signed rank test with a Bonferroni P value adjustment. PtGA, Fatigue, Pain, and Morning stiffness data are from daily survey; FSS and FACIT-F data are from weekday survey; and SF-36, PROMIS-29, SLAQ, and LupusPRO data are from weekly survey. FIG. 9B: Each bar represents a group of subjects based on self-reported ancestry. Differences in the mean rank of compliance across ancestries were insignificant for each survey type (Kruskal-Wallis analysis of variance; P>0.05). For each PRO, the thinner bars from left to right represent, African, Asian, and European and, and Hispanic Ancestry, the total is represented by the thick bar. FIG. 9C: Mean compliance is detailed weekly for each questionnaire during the 24-week trial and 2 weeks of baseline measures. For each ePRO, P values from a Wilcoxon signed rank test compare compliance between the 2-week baseline and weeks 23-24. At-2 week, lines from the top to the bottom are, PROMIS-29, SLAQ, morning stiffness, (PtGA, Fatigue, Pain), FSS, FACIT-F, SF-36, and Lupus PRO.

FIG. 10 shows distribution of electronic patient-reported outcome (ePRO) and paper PRO results for each survey. Boxplots showing the distribution and mean scores (blue text) for the indicated instrument. For multidomain PROs (Medical Outcomes Short Form 36 [SF-36], Patient Reported Outcome Measurement Information System [PROMIS-29], LuPRO), each point indicates an individual domain score for a particular patient.

FIG. 11 shows correlation between collection methods. Scatter plots showing the correlation between scores recorded by both collection methods. Pearson coefficients (R²) are shown in each plot. For multidomain patient-reported outcomes (PROs) (Medical Outcomes Short Form 36 [SF-36], Patient Reported Outcome Measurement Information System [PROMIS-29], LuPRO), each point indicates an individual domain score for a particular patient.

FIG. 12 shows Bland-Altman plots to assess agreement between collection methods. The difference between electronic patient-reported outcome [ePRO] score and the paper PRO score (ePRO−paper PRO) and the average of the patient global assessment (PtGA) scores (ePRO score+paper PRO score divided by 2) are represented on the y-axis and the x-axis, respectively. The top and bottom lines (e.g., along x-axis) represent the 95% confidence interval; the mean difference is in red text. For multidomain PROs (Medical Outcomes Short Form 36 [SF-36], Patient Reported Outcome Measurement Information System [PROMIS-29], LuPRO), each point indicates an individual domain score for a particular patient.

FIG. 13A-C show agreement between collection methods for the Functional Assessment of Chronic Illness Therapy-Fatigue Scale (FACIT-F) survey at selected timepoints. FIG. 13A: Boxplots showing the distribution and mean scores (blue text) for each administration method at baseline and at months 1, 3, and 6. FIG. 13B: Scatter plots showing the correlation between scores recorded by both collection methods. Pearson coefficients (R²) are shown in each plot. FIG. 13C: Bland-Atman plots were used to assess the agreement between each collection method. The difference between the electronic patient-reported outcome (ePRO) score and the paper PRO score (ePRO−paper PRO score) and the average (ePRO score+paper PRO score divided by 2) of the FACIT-F scores are represented on the y-axis and the x-axis, respectively. The top and bottom lines (e.g., along x-axis) represent the 95% confidence interval; the mean difference is in red text.

### DETAILED DESCRIPTION OF THE INVENTION

Patient-reported outcomes (PROs) data refer to a reported status of a patient's health conditions as reported by the patient without interpretation and/or supervision by a healthcare professional or other party. PROs may measure a person's perception of their own health, and may be reported by patients using questionnaires. For example, PROs may measure a patient's pain levels, fatigue, stiffness/flexibility, physical health, mental health, and the like. PRO data may be useful to healthcare product development, such as pharmaceutical and biologics product development, as patients may provide PROs that may be used to evaluate the safety and efficacy of products, for example.

Electronic Patient-reported outcomes (ePRO) data allow for real-time collection and assessment of PRO data in a patient's regular environment, as patient's may be able to report their status without needing to be in the presence of a healthcare professional. The collection of ePro data in clinical trials, for example, may offer advantages over some traditional paper-based methods. For example, ePROs are not location-dependent, may be conducted in an unsupervised manner, and allow for accurate and real-time reporting of symptoms. The real-time nature of ePRO data reduces the chances of intermittently collected data and of impatient patient recall.

In some embodiments, mobile device technology may be used to collect PRO data in an electronic format (ePRO). In particular, applications executed by mobile devices may present questionnaires with questions regarding a patient's health. The applications may receive patient inputs that indicate the patient's perception of their own health. In addition, mobile devices may collect biometric data (e.g., sleep data, breathing data, body temperature data, heart rate data, etc.), device motion data (e.g., indicative of activity, steps, etc.), and/or geo-location data from patients. The PRO and/or other data may be used assess a patient's health. In particular, the PRO and/or other data may be used to generate a score, and/or ii) an inference indicative of the disease state of the patient. In certain embodiments, the score is representative of a healthcare professional's evaluation of the patient. For example, the PRO and/or other data may be used to generate a physician's assessment score in an automated fashion, such as by using machine learning that inputs the PRO and/or biometric data, including ePRO and paper-administered PRO data, and generates a score that estimates what a physician would generate if a physician were analyzing the patient's data. For example, based on the inputs, such as the PRO and/or biometric data, including ePRO and/or paper-administered PRO data, the machine learning model may generate the inference indicative of the disease state of the patient. The inference can be whether the input data, such as ePRO data, is indicative of the patient having active disease or not having active disease. A patient having active disease may need further evaluation, consultation and/or treatment by a healthcare professional such as a physician. For example, to determine agreement between PRO information collected in different formats, intra-class correlation coefficients (ICCs), paired t-tests, and Band-Altman plots may be evaluated, along with compliance and Cronbach's alpha as a measure of survey reliability.

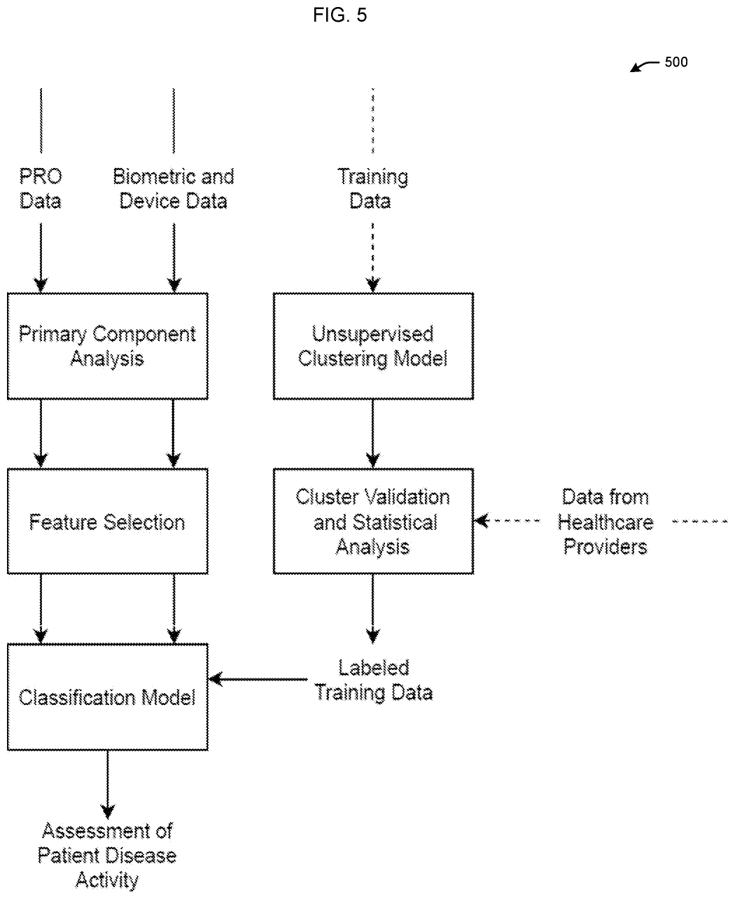

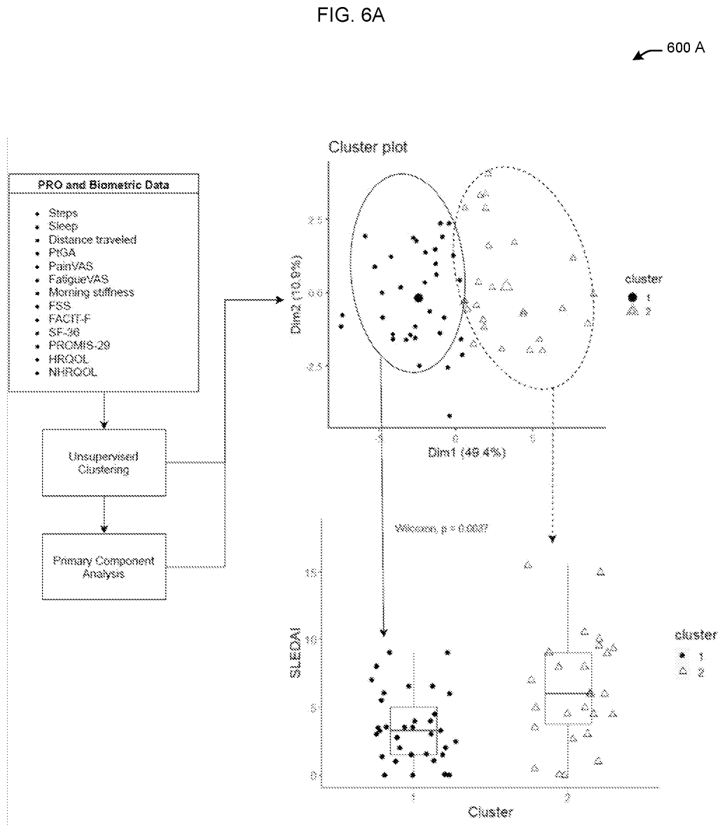

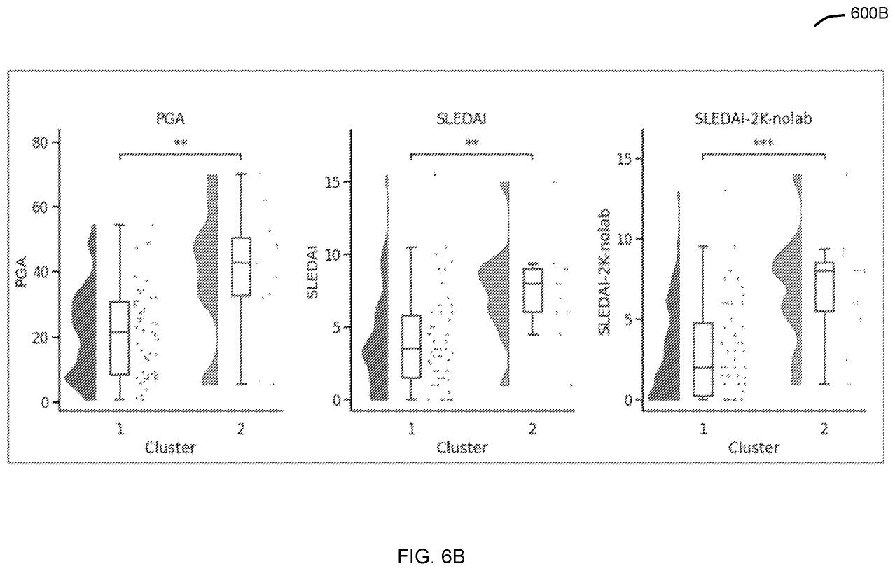

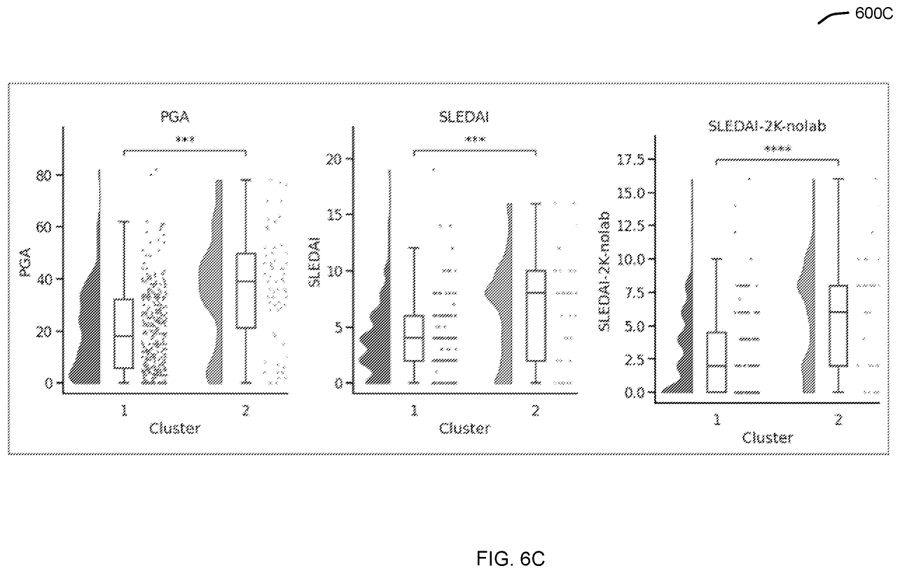

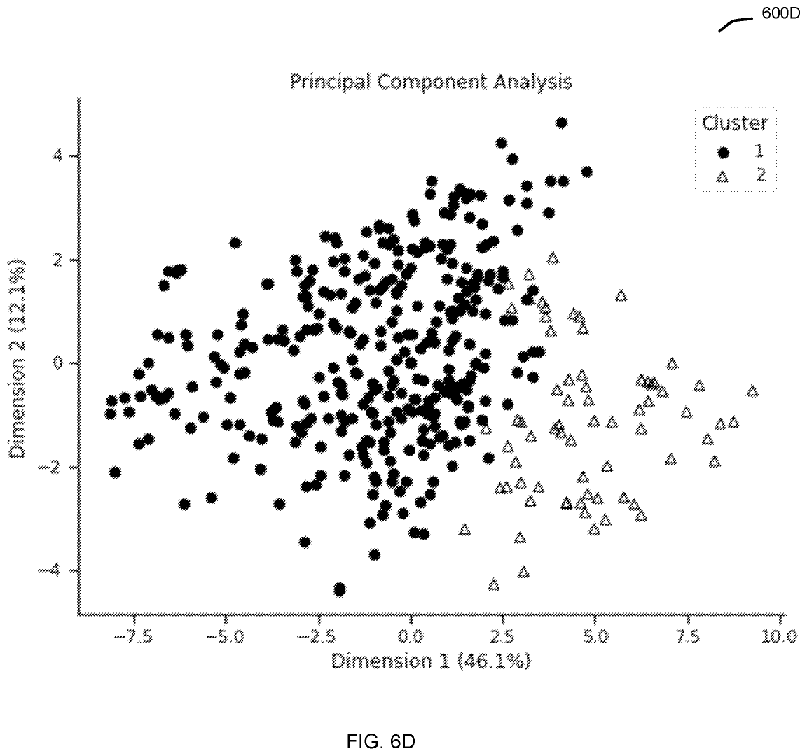

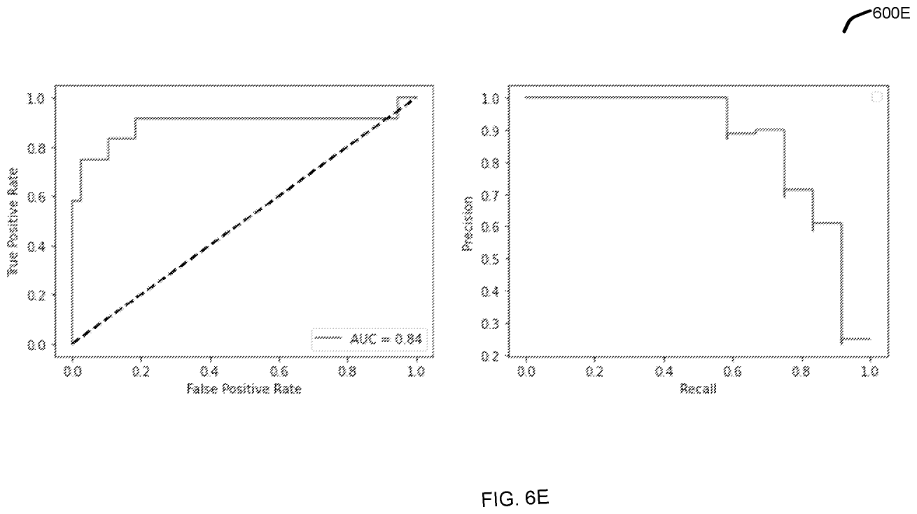

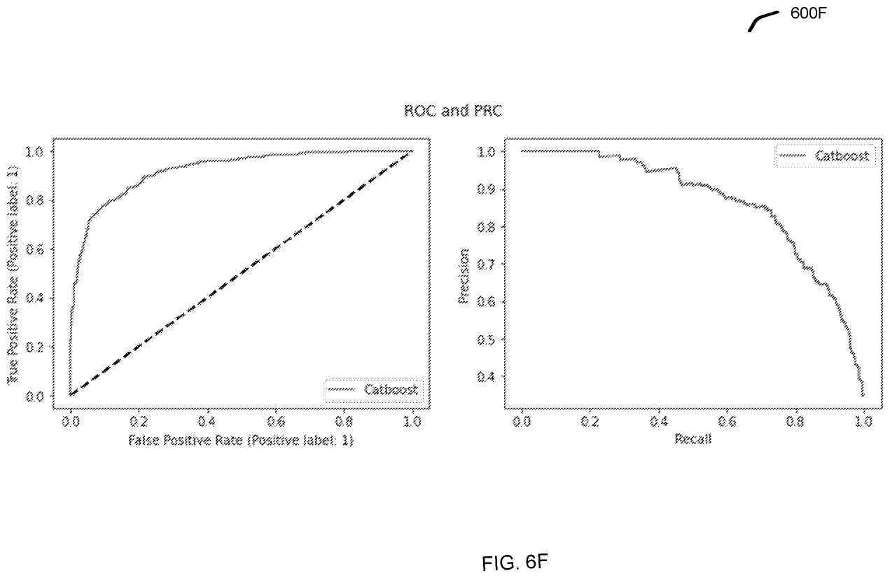

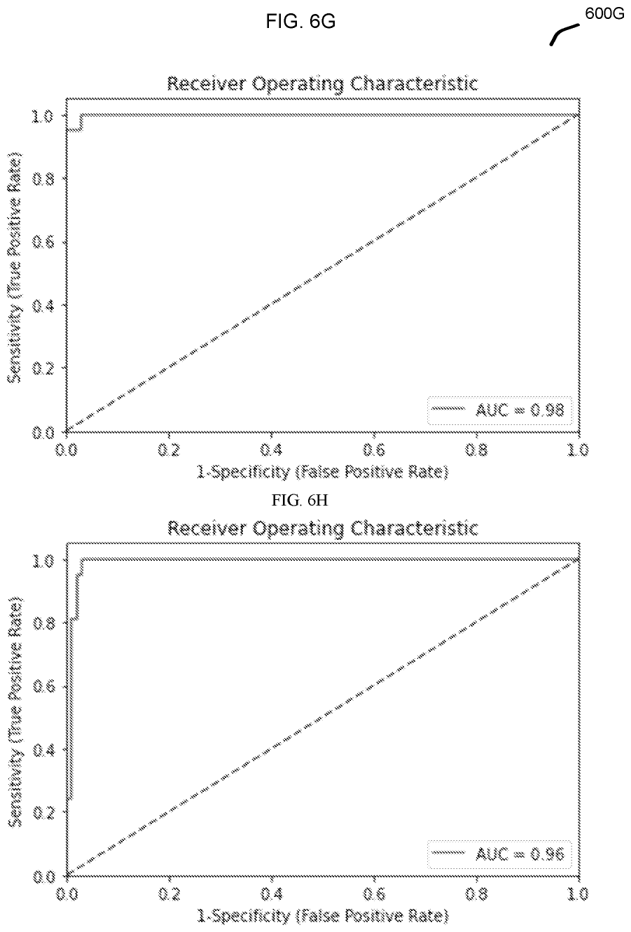

In some embodiments, a method of evaluation and/or treatment of a disease in a patient may include receiving, by a first device, PRO data from at least one other device, the PRO data including ePRO data and/or paper-administered data, indicating the patient's reactions i) to the patient's health, ii) to a method of evaluation, such as evaluation of the disease of the patient, and/or iii) to a method of treatment, such as treatment of the disease of the patient, using a pharmaceutical or biological treatment. In certain embodiments, the PRO data received is indicative of the patient's reactions to the patient's health. The patient's reactions to the patient's health may indicate patient's perception of their own health. In certain embodiments, the PRO data is ePRO data. ePRO can be collected in electronic format. The first device may validate the PRO data according to regulatory requirements. The first device may input the PRO data, optionally along with collected device and/or biometric data of the patient received from at least one other device, to a first machine learning model. In certain embodiments, the biometric data comprises sleep data, breathing data, body temperature data, heart rate data, or any combination thereof, of the patient. In certain embodiments, the device data comprises accelerometer data indicative of activity of the patient. In certain embodiments, the first device input the PRO data to the first machine learning model. The first machine learning model may include one or multiple layers that analyze the input data, and that may be trained to generate i) a score and/or ii) an inference indicative of the disease state of the patient. The score can be indicative of patient's health, what a healthcare professional would generate to assess the patient's health, the effectiveness of a current healthcare treatment, and/or severity the patient's reactions to the treatment. In certain embodiments, the first machine learning model is trained to generate the inference indicative of the disease state of the patient. The first device can contain a processor. The processor may receive the PRO data, and the optional device and/or biometric data; validate the PRO data; and input the PRO and optional data (e.g., if received) to the first machine learning model. The first machine learning model may generate the score and/or the inference, based on the input data (e.g., PRO data from, and optionally the device and/or biometric data from the at least one other device), and if trained, based on training data. The input data may optionally include data from the patient's visits to a doctor. The machine learning model can be trained using the training data. The training data can comprise PRO data, and optionally device and biometric data, from a plurality of reference patients. The training data may optionally include data from the reference patients' visit to doctors. In certain embodiments, the machine learning model is trained using a method described in the examples shown in FIG. 5 and/or FIGS. 6A-H. The first machine learning model can generate the score and/or the inference by comparing the input data with the training data. In certain embodiments, the first machine learning model is trained using the training data to generate the inference. The disease state of the patient can be the patient having the active disease, or not having active disease. The inference generated can be whether the input data, such as ePRO data, is indicative of the patient having active disease, or not having active disease. In certain embodiments, the score can be indicative of the patient having active disease or not having active disease. The method can classify the patient as having active disease, or not having active disease, based on the score and/or inference. The inference and the score can be a classification parameter, and the method can classify the patient based on the classification parameter, for example the inference generated can be that the input data, such as ePRO data, is indicative of the patient having active disease, and the method classify the patient having active disease. In certain embodiments, the score can be compared with a threshold score to classify the patient. A patient having active disease may need further evaluation, consultation and/or treatment by a healthcare professional, such as a physician. In certain embodiments, the method comprises training the first machine learning model to generate the score based on a percentage of scores associated with adjusting the first method of evaluation or treatment. The method can generate, recommend, and/or select based on the score and/or the inference, i) an action item, ii) a method of evaluation, and/or iii) a method of treatment for the patient. In certain embodiments, a second machine learning model or a layer of the first machine learning model generate, recommend, and/or select the action item, the method of evaluation, and/or the method of treatment for the patient. In certain embodiments, the action item, the method of evaluation, and/or the method of treatment for the patient, is generated, recommended, and/or selected based on a comparison of the score to a threshold score. The method of evaluation for the patient as generated, recommended and/or selected by the method may be a different method of evaluation for the patient than the method of evaluation for the patient being evaluated using the PRO data (e.g., if the score indicates a low effectiveness or high activity of the patient's disease/condition, e.g., patient having active disease), or may be the same method of evaluation for the patient as the method of evaluation for the patient being evaluated using the PRO data (e.g., when the score indicates a high effectiveness of the current method of evaluation and/or treatment or a low activity of the patient's disease/condition, e.g., patient not having the active disease). The method of treatment for the patient as generated, recommended and/or selected by the method may be a different method of treatment for the patient than the method of treatment for the patient being evaluated using the PRO data (e.g., if the score indicates a low effectiveness or high activity of the patient's disease/condition, e.g., patient having active disease), or may be the same method of treatment for the patient as the method of treatment for the patient being evaluated using the PRO data (e.g., when the score indicates a high effectiveness of the current method of evaluation and/or treatment or a low activity of the patient's disease/condition, e.g., patient not having the active disease). In this manner, generating a new/different method of evaluation and/or treatment for the patient may be optional. In certain embodiments, for a patient classified as having the active disease, the action item generated, recommended and/or selected, can include scheduling an appointment, visit, and/or consultation for the patient with a healthcare professional, such as a physician. The appointment, visit, and/or consultation can be between the healthcare professional, and the patient and/or a party responsible for the patient, and can be in-person, online, telephonic (e.g., telemedicine), and/or the like appointment, visit, and/or consultation. In certain embodiments, for a patient classified as having the active disease, the action item generated, recommended and/or selected, can include sending the patient's classification, score and/or inference to a healthcare professional, with provisions for requirement of immediate attention. For a patient classified as having the active disease, the method of evaluation generated, recommended and/or selected can include further test for the disease. The further test can include one or more tests, such as one or more genetic, blood, and/or laboratory tests, for determining type of the disease, such as endotype of the disease the patient has; severity of the disease in the patient; and/or suitable treatment of the disease for the patient. The further test may be performed by a healthcare professional. The treatment (e.g., the treatment generated, recommended and/or selected by the method) can be one or more treatments of the disease. In certain embodiments, the treatment can include a pharmaceutical composition. In certain embodiments, the treatment can treat, reduce severity of, and/or reduce risk of having the disease, such as active disease. In certain embodiments, method includes performing the action item, performing the method of evaluation, and/or administering the treatment to the patient. In certain embodiments, a healthcare professional based on the score, inference, classification, and/or action item may generate, recommend, select, and/or perform the method of evaluation, and/or the method of treatment. In certain embodiments, the method includes performing the action item. In certain embodiments, method includes performing the method of evaluation. In certain embodiments, method includes administering the treatment (e.g., the treatment generated, recommended and/or selected by the method) to the patient.

The disease may be a disease or condition in which evaluation of PRO data can indicate the presence of and/or the progression of disease. It is understood that PRO data may be reported by an individual monitoring the patient, for example, a parent or caregiver. The disease or condition may be selected from, e.g., an inflammatory (autoimmune) condition as described herein, a cardiovascular disease or condition, a neurological disease or condition, a developmental disease or disorder, a degenerative disease or disorder, a kidney disease or condition, and/or a cancer. In related embodiments, the PRO data received may be any known to those of skill in the art relating to the disease or condition, including but not limited to data obtained using a general wellness questionnaire (e.g., PROMIS-29 or SF-36). The cardiovascular disease or condition may be heart attack, stroke, coronary heart disease, cerebrovascular disease, peripheral arterial disease (PAD), rheumatic heart disease, congenital heart disease, deep vein thrombosis (DVT), heart arrythmia, heart failure, hypertensive heart disease, valvular heart disease, carditis, pericarditis, aortic aneurysm, venous thrombosis, thromboembolic disease, cardiomyopathy, myocardial infarction, coronary atherosclerosis, angina, pulmonary embolism, heart cancer, or any other disease or condition that compromises heart function. In certain embodiment, the PRO data received may be any known to those of skill in the art relating to the cardiovascular disease or condition, including but not limited to data obtained using a general wellness questionnaire. In some embodiments, the PRO data received in relation to the cardiovascular disease or condition is selected from data obtained using a questionnaire selected from one or more of the Seattle Angina Questionnaire (SAQ)-7, the Rose Dyspnea Scale, HRQOL and Patient Health Questionnaire-2, SF-12, SF-36, Euro-Qol-5D (EQ-5D), PROMIS-Global Health, Kansas City Cardiomyopathy Questionnaire (KCCQ), and Minnesota Living with Heart Failure Questionnaire (MLHFQ), Fatigue VAS, Pain VAS, PtGA, FSS, FACIT-F, Morning Stiffness, PROMIS-29, and SF-36. Instruments for obtaining patient data relating to cardiovascular health are known in the art and described in the literature, e.g., by R. Kornowski, 2021, European Heart Journal-Quality of Care and Clinical Outcomes, 0:1-9, https://doi.org/10.1093/ehjqcco/qcab051, and A. A. Kelkar, 2016, Am Coll Cardiol HF 2016;4:165-75, each incorporated herein by reference in its entirety. A neurological disease or condition may be any that compromises neurological function, e.g., by affecting the brain, spinal cord, and/or nerves. For example, the neurological disease or condition may be autism, acute spinal cord injury, Alzheimer's disease, amyotrophic lateral sclerosis (als), ataxia, Bell's palsy, brain tumors, cerebral aneurysm, epilepsy and seizures, Guillain-Barré syndrome, headache, head injury, lumbar disk disease (herniated disk), multiple sclerosis, muscular dystrophy, neurocutaneous syndromes, Parkinson's disease, a psychological disorder (e.g., anxiety, depression, bipolar disorder, schizophrenia, PTSD, suicidal ideation), stroke (brain attack), concussion, cluster headaches, tension headaches, or migraine headaches. In certain embodiments, the PRO data received may be any known to those of skill in the art relating to the neurological disease or condition, including but not limited to data obtained using a general wellness questionnaire (e.g., PROMIS-29 or SF-36). In these embodiments as well as the others described herein, when evaluated over time, a change or trend in disease severity as indicated by the score and/or inference may predict or indicate the onset or presence of a disease event, e.g., a neurological event. For example, a psychological break or other neurological disease event may be predicted or detected and an appropriate intervention applied by a healthcare provider. In some embodiments, the PRO data received in relation to the kidney disease or condition is selected from data obtained using a questionnaire selected from one or more of Neuro-QOL, Patient Health Questionnaire (PHQ-9), Epworth Sleepiness Scale (ESS), Epworth Sleepiness Scale for Children and Adolescents (ESS-CHAD), Generalised Anxiety Disorder Assessment (GAD-7), and Seizure Severity Questionnaire (SSQ). A developmental disease or disorder may be any that arises and potentially progresses during development, including but not limited to those set forth herein. A degenerative disease or disorder may be any that progresses over time, including but not limited to those set forth herein. In at least these embodiments, the PRO data may be provided by an individual caring for the patient. A kidney disease or condition may be any that compromises kidney function, e.g., chronic kidney disease (CKD), polycystic kidney disease (PKD), acute kidney injury (AKI), kidney stones, kidney infection, kidney cancer, or kidney cysts. In certain embodiments, the PRO data received may be any known to those of skill in the art relating to the kidney disease or condition, including but not limited to data obtained using a general wellness questionnaire (e.g., PROMIS-29 or SF-36). In some embodiments, the PRO data received in relation to the kidney disease or condition is selected from data obtained using a questionnaire selected from one or more of Dialysis Symptom Index (DSI), Palliative Care Outcome Scale-Renal Version (IPOS-Renal), Kidney Disease Quality of Life Instrument (KDQOL-SF), PRO-Kid, and any other known instrument, e.g., as evaluated by E. M. van der Willik, et al., 2019, BMC Nephrology 20:344, incorporated herein by reference in its entirety. A cancer may be any cancer, e.g., a solid tumor or a blood cancer (e.g., leukemia, lymphoma, myeloma). A cancer may be selected from but not limited to cancer of the lung, liver, pancreas, brain, ovary, lymph node, skin, breast, GI tract (e.g., esophageal, colon, stomach, ileum, duodenum, rectum, anus), uterus, cervix, bladder, prostate, head and neck, and bone. In certain embodiments, the PRO data received may be any known to those of skill in the art relating to the cancer, including but not limited to data obtained using a general wellness questionnaire (e.g., PROMIS-29 or SF-36). In some embodiments, the PRO data received in relation to the cancer is selected from data obtained using a questionnaire selected from one or more of FACT-G, PRO-CTCAE, PATIENTVIEWPOINT, PATIENT CARE MONITOR (PCM), ADVANCED SYMPTOM MANAGEMENT SYSTEM (ASyMS), SYMPTOM TRACKING AND REPORTING (STAR) PROSTATECTOMY PROJECT, TELL US, and any other known instrument, e.g., as described by A. V. Bennett, et al., 2012, CA Cancer Journal for Clinicians 62:336-347, incorporated herein by reference in its entirety.

In some embodiments, the disease is an autoimmune disease. Non-limiting examples of the autoimmune disease can include lupus, lupus nephritis, rheumatoid arthritis, Sjögren syndrome, inflammatory bowel disease (e.g., Crohn's disease, ulcerative colitis), vitiligo, polymyositis, pemphigus, autoimmune hepatitis, hypopituitarism, myocarditis, autoimmune skin diseases (e.g., atopic dermatitis, psoriasis, scleroderma), autoimmune vasculitis, Addison's disease, celiac disease, dermatomyositis, Graves disease, Hashimoto's thyroiditis, multiple sclerosis, myasthenia gravis, pernicious anemia, reactive arthritis, and type I diabetes. In certain embodiment, the PRO data received may be any known to those of skill in the art relating to the autoimmune disease, including but not limited to data obtained using a general wellness questionnaire. In certain embodiments, the PRO data received in relation to autoimmune disease includes one or more of SLAQ data, HRQOL data, Non-HRQOL data, Fatigue VAS data, Pain VAS data, PtGA data, FSS data, FACIT-F data, Morning Stiffness data, PROMIS-29 data, and SF-36 data. In certain embodiments, the PRO data received in relation to autoimmune disease includes one or more of Fatigue VAS data, Pain VAS data, PtGA data, FSS data, FACIT-F data, Morning Stiffness data, PROMIS-29 data, and SF-36 data. PRO data can include patient's response to the PRO questionnaires, for example FSS data includes patient's response to the FSS questionnaires. In certain embodiments, PROMIS-29 data can include Fatigue data, Sleep disturbance data, Depression data, Anxiety data, Pain Intensity data, Pain interference data, Satisfaction with social role data, physical function data, or any combination thereof. In certain embodiments, SF-36 data can include vitality, bodily pain, general health, mental health, physical function, role emotional, role physical, social function or any combination thereof. For each disease or disorder, the evaluation, such as the score and/or inference may indicate, suggest and/or predict the presence or absence of the disease or condition. In the known (previously identified) presence of the disease or condition, the score and/or inference may indicate, suggest and/or predict more severe disease or less severe disease. In certain embodiments, the evaluation, such as the score and/or inference may indicate, suggest and/or predict the presence or absence of disease flare or pre-flare state, such as autoimmune disease flare, in the patient. In certain embodiments, a patient classified as having active disease suggest or indicate that the patient has the disease. In certain embodiments, a patient classified as not having active disease suggest or indicate that the patient does not have the disease. In certain embodiments, a patient classified as having active disease suggest or indicate that the patient has more severe disease state. In certain embodiments, a patient classified as not having active disease suggest or indicate that the patient has less severe disease state. In certain embodiments, a patient classified as having active disease suggest or indicate that the patient is in a pre-flare state, or experiencing a disease flare. In certain embodiments, a patient classified as not having active disease suggest or indicate that the patient is not in a pre-flare state, or not experiencing a disease flare. A patient in a pre-flare state may experience disease flare in 1 day, 2 days, 3 days, 4 days, 5 days, 6 days, 1 week, 2 weeks, 3 weeks, in a month, or any time or range therebetween.

The suggestion or identification of a more severe disease state in such a patient may indicate that the patient could benefit from prompt intervention, e.g., treatment with a drug, a change in treatment, and/or further evaluation. In some embodiments, in the context of a drug or treatment clinical trial, the suggestion or identification of a more severe or less severe disease state in such a patient may indicate the level of effectiveness of the drug for the particular patient. For example, the suggestion or identification of a more severe disease state in a patient may indicate that the drug is not effective in treating the disease or disorder. Conversely, the suggestion or identification of a less severe disease state in a patient may indicate that the drug is effective in treating the disease or disorder. In some embodiments, the patient has no prior diagnosis of the disease or condition. The suggestion or identification of a disease or condition in such a patient may indicate that the patient could benefit from prompt intervention, e.g., treatment with a drug and/or further evaluation.

In some embodiments, a patient (previously diagnosed with, suspected of having, or never diagnosed with, the disease or disorder) that is evaluated for a cardiovascular disease or condition and identified as having severe disease may be a patient currently experiencing a cardiac event, or likely to experience a cardiac event, such as in 1-72 hours. In some embodiments, the patient is identified as likely to experience a cardiac event in 1 hour, 3 hours, 6 hours, 12 hours, 24 hours, 2 days, 5 days, 1 week, 2 weeks, 1 month, 3 months or 6 months, or any time or range there between. Non-limiting examples of a cardiac event can include heart failure, heart attack, arrhythmia, valve disease, high blood pressure, chest pain or discomfort, and shortness of breath.

In some embodiments, a patient (previously diagnosed with, suspected of having, or never diagnosed with, the disease or disorder) that is evaluated for a neurological disease or condition and identified as having severe disease may be a patient currently experiencing a neurological event, or likely to experience a neurological event, such as in 1-72 hours. In some embodiments, the patient is identified as likely to experience a cardiac event in 1 hour, 3 hours, 6 hours, 12 hours, 24 hours, 2 days, 5 days, 1 week, 2 weeks, 1 month, 3 months or 6 months, or any time or range there between. Non-limiting examples of a neurological event can include seizure, a psychological break, or suicide. In some embodiments the neurological event may be the onset of a neurological condition, e.g., a diagnosis of autism spectrum disorder. In these embodiments the evaluation results may be conveyed to a healthcare provider who may recommend any known intervention for preventing or mitigating the neurological event.

In some embodiments, a patient (previously diagnosed with, suspected of having, or never diagnosed with, the disease or disorder) that is evaluated for a kidney disease or condition and identified as having severe disease may be a patient currently experiencing a kidney event, or likely to experience a kidney event or have kidney damage. In some embodiments, the patient is identified as likely to experience a kidney event in 1-72 hour, 1 hour, 3 hours, 6 hours, 12 hours, 24 hours, 2 days, 5 days, 1 week, 2 weeks, 1 month, 3 months or 6 months, or any time or range therebetween. Non-limiting examples of a kidney event can include kidney failure, e.g., occurrence of a GFR of below 15 milliliters per minute, or reduced kidney function, e.g., occurrence of a GFR of below 60 milliliters per minute, from 15 to 60 milliliters per minute, or any range or value therebetween. In some embodiments, the patient is identified as likely to have kidney damage, e.g., occurrence of Albumin to Creatinine Ratio of greater than 30 mg/g (A1), 30-300 mg/g (A2), or greater than 300 mg/g (A3).

In some embodiments, a patient (previously diagnosed with, suspected of having, or never diagnosed with the cancer) that is evaluated for a cancer and identified as having severe disease may be a patient currently experiencing cancer proliferation, including but not limited to growth of a new cancer, enlargement or increased number of tumors in an existing cancer, or metastasis.

A patient having autoimmune disease may be classified to suggest or indicate or predict flare status. In some embodiments, a patient (previously diagnosed with, suspected of having, or never diagnosed with, the disease or disorder) that is evaluated for an autoimmune disease or condition and classified as having active disease may be a patient in a pre-flare state, or experiencing a disease flare. In certain embodiments, the disease is an autoimmune disease, and the classification of a patient as having active disease suggests or indicates that the patient is in a pre-flare state, or experiencing a disease flare, and the classification of a patient as not having active disease suggests or indicates that the patient is not in a pre-flare state, or not experiencing a disease flare.

In certain embodiments, the patient is suspected of having the disease. In certain embodiments, the patient is asymptomatic of the disease. The first machine learning model can generate the score and/or inference using linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof. The algorithm of the first machine learning model can be a machine learning classifier mentioned in this paragraph. The machine learning classifier (e.g., linear regression, LOG, Ridge regression, Lasso regression, EN regression, SVM, GBM, kNN, GLM, NB classifier, neural network, a RF, deep learning algorithm, LDA, DTREE, ADB, CART, and/or hierarchical clustering) can be trained to obtain the first machine learning model. In some embodiments, the first machine learning model, is trained (e.g., obtained by training) using a supervised machine learning algorithm or an unsupervised machine learning algorithm, e.g., the classifier can be a supervised machine learning algorithm or an unsupervised machine learning algorithm. In certain embodiments, the first machine learning model generate the score and/or inference using linear regression. In certain embodiments, the first machine learning model generate the score and/or inference using LOG. In certain embodiments, the first machine learning model generate the score and/or inference using Ridge regression. In certain embodiments, the first machine learning model generate the score and/or inference using Lasso regression. In certain embodiments, the first machine learning model generate the score and/or inference using EN regression. In certain embodiments, the first machine learning model generate the score and/or inference using SVM. In certain embodiments, the first machine learning model generate the score and/or inference using GBM. In certain embodiments, the first machine learning model generate the score and/or inference using kNN. In certain embodiments, the first machine learning model generate the score and/or inference using GLM. In certain embodiments, the first machine learning model generate the score and/or inference using NB classifier. In certain embodiments, the first machine learning model generate the score and/or inference using neural network. In certain embodiments, the first machine learning model can generate the score and/or inference using RF. In certain embodiments, the first machine learning model generate the score and/or inference using deep learning algorithm. In certain embodiments, the first machine learning model generate the score and/or inference using LDA. In certain embodiments, the first machine learning model can generate the score and/or inference using DTREE. In certain embodiments, the first machine learning model generate the score and/or inference using ADB. In certain embodiments, the first machine learning model generate the score and/or inference using CART. In certain embodiments, the first machine learning model generate the score and/or inference using hierarchical clustering.

The first machine learning model can have a receiver operating characteristic (ROC) curve with an Area-Under-Curve (AUC) of at least about 0.7, at least about 0.75, at least about 0.8, at least about 0.85, at least about 0.9, at least about 0.91, at least about 0.92, at least about 0.93, at least about 0.94, at least about 0.95, at least about 0.96, at least about 0.97, at least about 0.98, at least about 0.99, or more than about 0.99. In certain embodiments, the first machine learning model has a ROC curve with an AUC of about 0.8 to about 1. In certain embodiments, the first machine learning model has a ROC curve with an AUC of about 0.8 to about 0.85, about 0.8 to about 0.9, about 0.8 to about 0.92, about 0.8 to about 0.93, about 0.8 to about 0.94, about 0.8 to about 0.95, about 0.8 to about 0.96, about 0.8 to about 0.97, about 0.8 to about 0.98, about 0.8 to about 0.99, about 0.8 to about 1, about 0.85 to about 0.9, about 0.85 to about 0.92, about 0.85 to about 0.93, about 0.85 to about 0.94, about 0.85 to about 0.95, about 0.85 to about 0.96, about 0.85 to about 0.97, about 0.85 to about 0.98, about 0.85 to about 0.99, about 0.85 to about 1, about 0.9 to about 0.92, about 0.9 to about 0.93, about 0.9 to about 0.94, about 0.9 to about 0.95, about 0.9 to about 0.96, about 0.9 to about 0.97, about 0.9 to about 0.98, about 0.9 to about 0.99, about 0.9 to about 1, about 0.92 to about 0.93, about 0.92 to about 0.94, about 0.92 to about 0.95, about 0.92 to about 0.96, about 0.92 to about 0.97, about 0.92 to about 0.98, about 0.92 to about 0.99, about 0.92 to about 1, about 0.93 to about 0.94, about 0.93 to about 0.95, about 0.93 to about 0.96, about 0.93 to about 0.97, about 0.93 to about 0.98, about 0.93 to about 0.99, about 0.93 to about 1, about 0.94 to about 0.95, about 0.94 to about 0.96, about 0.94 to about 0.97, about 0.94 to about 0.98, about 0.94 to about 0.99, about 0.94 to about 1, about 0.95 to about 0.96, about 0.95 to about 0.97, about 0.95 to about 0.98, about 0.95 to about 0.99, about 0.95 to about 1, about 0.96 to about 0.97, about 0.96 to about 0.98, about 0.96 to about 0.99, about 0.96 to about 1, about 0.97 to about 0.98, about 0.97 to about 0.99, about 0.97 to about 1, about 0.98 to about 0.99, about 0.98 to about 1, or about 0.99 to about 1. In certain embodiments, the first machine learning model has a ROC curve with an AUC of about 0.8, about 0.85, about 0.9, about 0.92, about 0.93, about 0.94, about 0.95, about 0.96, about 0.97, about 0.98, about 0.99, or about 1. In certain embodiments, the first machine learning model has a ROC curve with an AUC of at least about 0.8, about 0.85, about 0.9, about 0.92, about 0.93, about 0.94, about 0.95, about 0.96, about 0.97, about 0.98, or about 0.99. In certain embodiments, the first machine learning model has a ROC curve with an AUC of about 0.6 to about 1. In certain embodiments, the first machine learning model has a ROC curve with an AUC of about 0.6 to about 0.65, about 0.6 to about 0.7, about 0.6 to about 0.75, about 0.6 to about 0.8, about 0.6 to about 0.85, about 0.6 to about 0.9, about 0.6 to about 0.95, about 0.6 to about 1, about 0.65 to about 0.7, about 0.65 to about 0.75, about 0.65 to about 0.8, about 0.65 to about 0.85, about 0.65 to about 0.9, about 0.65 to about 0.95, about 0.65 to about 1, about 0.7 to about 0.75, about 0.7 to about 0.8, about 0.7 to about 0.85, about 0.7 to about 0.9, about 0.7 to about 0.95, about 0.7 to about 1, about 0.75 to about 0.8, about 0.75 to about 0.85, about 0.75 to about 0.9, about 0.75 to about 0.95, about 0.75 to about 1, about 0.8 to about 0.85, about 0.8 to about 0.9, about 0.8 to about 0.95, about 0.8 to about 1, about 0.85 to about 0.9, about 0.85 to about 0.95, about 0.85 to about 1, about 0.9 to about 0.95, about 0.9 to about 1, or about 0.95 to about 1. In certain embodiments, the first machine learning model has a ROC curve with an AUC of about 0.6, about 0.65, about 0.7, about 0.75, about 0.8, about 0.85, about 0.9, about 0.95, or about 1. In certain embodiments, the first machine learning model has a ROC curve with an AUC of at least about 0.6, about 0.65, about 0.7, about 0.75, about 0.8, about 0.85, about 0.9, or about 0.95.

The method can classify whether the patient has active disease, or does not have active disease with an accuracy of about 80% to about 100%. The method can classify whether the patient has active disease, or does not have active disease with an accuracy of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active disease, or does not have active disease with an accuracy of about 80%, about 85%, about 90%, about 92%, about 93%, about 94%, about 95%, about 96%, about 97%, about 98%, about 99%, or about 100%. The method can classify whether the patient has active disease, or does not have active disease with an accuracy of at least about 80%, about 85%, about 90%, about 92%, about 93%, about 94%, about 95%, about 96%, about 97%, about 98%, or about 99%. In certain embodiments, the method classify whether the patient has active disease, or does not have active disease with an accuracy of about 60% to about 100%. In certain embodiments, the method classify whether the patient has active disease, or does not have active disease with an accuracy of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active disease, or does not have active disease with an accuracy of about 60%, about 65%, about 70%, about 75%, about 80%, about 85%, about 90%, about 95%, or about 100%. In certain embodiments, the method classify whether the patient has active disease, or does not have active disease with an accuracy of at least about 60%, about 65%, about 70%, about 75%, about 80%, about 85%, about 90%, or about 95%.

The method can classify whether the patient has active disease, or does not have active disease with a sensitivity of about 80% to about 100%. The method can classify whether the patient has active disease, or does not have active disease with a sensitivity of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active disease, or does not have active disease with a sensitivity of about 80%, about 85%, about 90%, about 92%, about 93%, about 94%, about 95%, about 96%, about 97%, about 98%, about 99%, or about 100%. The method can classify whether the patient has active disease, or does not have active disease with a sensitivity of at least about 80%, about 85%, about 90%, about 92%, about 93%, about 94%, about 95%, about 96%, about 97%, about 98%, or about 99%. In certain embodiments, the method classify whether the patient has active disease, or does not have active disease with a sensitivity of about 60% to about 100%. In certain embodiments, the method classify whether the patient has active disease, or does not have active disease with a sensitivity of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active disease, or does not have active disease with a sensitivity of about 60%, about 65%, about 70%, about 75%, about 80%, about 85%, about 90%, about 95%, or about 100%. In certain embodiments, the method classify whether the patient has active disease, or does not have active disease with a sensitivity of at least about 60%, about 65%, about 70%, about 75%, about 80%, about 85%, about 90%, or about 95%.

The method can classify whether the patient has active disease, or does not have active disease with a specificity of about 80% to about 100%. The method can classify whether the patient has active disease, or does not have active disease with a specificity of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with a specificity of about 80%, about 85%, about 90%, about 92%, about 93%, about 94%, about 95%, about 96%, about 97%, about 98%, about 99%, or about 100%. The method can classify whether the patient has active disease, or does not have active disease with a specificity of at least about 80%, about 85%, about 90%, about 92%, about 93%, about 94%, about 95%, about 96%, about 97%, about 98%, or about 99%. In certain embodiments, the method classify whether the patient has active disease, or does not have active disease with a specificity of about 60% to about 100%. In certain embodiments, the method classify whether the patient has active disease, or does not have active disease with a specificity of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active disease, or does not have active disease with a specificity of about 60%, about 65%, about 70%, about 75%, about 80%, about 85%, about 90%, about 95%, or about 100%. In certain embodiments, the method classify whether the patient has active disease, or does not have active disease with a specificity of at least about 60%, about 65%, about 70%, about 75%, about 80%, about 85%, about 90%, or about 95%.

The method can classify whether the patient has active disease, or does not have active disease with a positive predictive value of about 80% to about 100%. The method can classify whether the patient has active disease, or does not have active disease with a positive predictive value of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active disease, or does not have active disease with a positive predictive value of about 80%, about 85%, about 90%, about 92%, about 93%, about 94%, about 95%, about 96%, about 97%, about 98%, about 99%, or about 100%. The method can classify whether the patient has active disease, or does not have active disease with a positive predictive value of at least about 80%, about 85%, about 90%, about 92%, about 93%, about 94%, about 95%, about 96%, about 97%, about 98%, or about 99%. In certain embodiments, the method classify whether the patient has active disease, or does not have active disease with a positive predictive value of about 60% to about 100%. In certain embodiments, the method classify whether the patient has active disease, or does not have active disease with a positive predictive value of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active disease, or does not have active disease with a positive predictive value of about 60%, about 65%, about 70%, about 75%, about 80%, about 85%, about 90%, about 95%, or about 100%. In certain embodiments, the method classify whether the patient has active disease, or does not have active disease with a positive predictive value of at least about 60%, about 65%, about 70%, about 75%, about 80%, about 85%, about 90%, or about 95%.

The method can classify whether the patient has active disease, or does not have active disease with a negative predictive value of about 80% to about 100%. The method can classify whether the patient has active disease, or does not have active disease with a negative predictive value of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active disease, or does not have active disease with a negative predictive value of about 80%, about 85%, about 90%, about 92%, about 93%, about 94%, about 95%, about 96%, about 97%, about 98%, about 99%, or about 100%. The method can classify whether the patient has active disease, or does not have active disease with a negative predictive value of at least about 80%, about 85%, about 90%, about 92%, about 93%, about 94%, about 95%, about 96%, about 97%, about 98%, or about 99%. In certain embodiments, the method classify whether the patient has active disease, or does not have active disease with a negative predictive value of about 60% to about 100%. In certain embodiments, the method classify whether the patient has active disease, or does not have active disease with a negative predictive value of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active disease, or does not have active disease with a negative predictive value of about 60%, about 65%, about 70%, about 75%, about 80%, about 85%, about 90%, about 95%, or about 100%. In certain embodiments, the method classify whether the patient has active disease, or does not have active disease with a negative predictive value of at least about 60%, about 65%, about 70%, about 75%, about 80%, about 85%, about 90%, or about 95%.

In some embodiments, a method for predicting the Physician's Global Assessment Score may include receiving, by a first device, PRO data from at least one other device, the PRO data including ePRO data and/or paper-administered data, indicating patient reactions to their health, and/or patient reactions to a method of treatment using a pharmaceutical or biological treatment. The first device may validate the PRO data according to regulatory requirements. The first device may receive documents with patient data from patient visits to a doctor. The first device may input the PRO data and the documents to a first machine learning model, which may generate, based on the PRO data and the documents, a prediction of a Physician's Global Assessment Score indicative of a patient's status or the severity of the patient's reactions. The method may generate, recommend, and/or select, based on the prediction of the Physician's Global Assessment Score, a second method of evaluation or treatment for the patient. In certain embodiments, a second machine learning model or layer of the first machine learning model may generate, recommend, and/or select the second method of treatment for the patient. The method of evaluation and/or treatment for the patient as generated by method may be a different method of evaluation and/or treatment for the patient than the method of evaluation and/or treatment for the patient being evaluated using the PRO data (e.g., if the Physician's Global Assessment Score indicates a low effectiveness or high activity of the patient's disease/condition), or may be the same method of evaluation and/or treatment for the patient as the method of evaluation and/or treatment for the patient being evaluated using the PRO data (e.g., when the Physician's Global Assessment Score indicates a high effectiveness of the current method of evaluation and/or treatment or a low activity of the patient's disease/condition).

Certain embodiments are directed to a method of diagnosis and/or treatment of lupus in a patient. The method may include receiving, by a first device, PRO data from a second device, the PRO data indicative of patient's reactions i) to the patient's health, ii) to a method of evaluation, such as evaluation of lupus of the patient, and/or iii) to a method of treatment, such as treatment of lupus of the patient, using a pharmaceutical or biological treatment. In certain embodiments, the PRO data received is indicative of the patient's reactions to the patient's health. The patient's reactions to the patient's health may indicate patient's perception of their own health. The PRO data can include ePRO data and/or paper-administered data. In certain embodiments, the PRO data comprises ePRO data. The first device may validate the PRO data according to regulatory requirements. The first device may optionally receive data from the patient's visits to a doctor. The first device may optionally receive biometric and/or device data of the patient. Non-limiting examples of biometric data can include one or more of sleep data, breathing data, body temperature data, heart rate data, or the like, of the patient. Non-limiting examples of device data can include accelerometer data, or the like. The first device may input the PRO data and the optional data (if received), to a first machine learning model, which may generate, based on the input data (e.g., the PRO data and the optional data (if received)), i) a score indicative of activity of the patient's disease (e.g., lupus) and reactions, and/or ii) an inference indicative of the lupus disease state of the patient. The optional data can include i) the doctor's data (e.g., the data from the patient's visits to the doctor), ii) biometric data, and/or iii) the device data. In certain embodiment, first machine learning model, generate, based on the input data, such as ePRO data, the inference indicative of the lupus disease state of the patient. The first device can contain a processor, and the processor may receive the PRO data, and the optional data, validate the PRO data, and input the PRO and optional data (e.g., if received) to the first machine learning model. In certain embodiments, the score can be a predicted Systemic Lupus Erythematosus Disease Activity Index (SLEDAI) score for the patient. The first machine learning model may generate the score and/or the inference based on the input data, and if trained, based on training data. The first machine learning model can generate the score and/or inference based on comparing the PRO data with the training data. The first machine learning model can be trained using the training data. The training data can comprise PRO data from a plurality of reference patients, and optionally i) data from the reference patients' visit to doctors, ii) biometric data from the plurality of reference patients, and/or iii) device data from the plurality of reference patients. In certain embodiments, the training data comprises PRO data from the plurality of reference patients. In certain embodiments, the first machine learning model is trained using a method described in the examples of FIG. 5 and/or FIGS. 6A-H. In certain embodiments, the first machine learning model is trained using the training data to generate the inference. The lupus disease state of the patient can be the patient having the active lupus, or not having active lupus. The inference generated can be whether the input data, such as ePRO data, is indicative of the patient having active lupus, or not having active lupus. In certain embodiments, the score can be indicative of the patient having active lupus or not having active lupus. The method can classify a patient as having active lupus, or not having active lupus, based on the score and/or inference. The inference and the score can be a classification parameter, and the method can classify the patient based on the classification parameter, for example the inference generated can be that the input data, such as ePRO data, is indicative of the patient having active lupus, and the method classify the patient having active lupus. In certain embodiments, the score can be compared with a threshold score to classify the patient. A patient classified as having active lupus suggest or indicate that the patient in a lupus pre-flare state, or experiencing lupus flare. A patient classified as not having active lupus suggest or indicate that the patient is not in a pre-flare state, or not experiencing lupus flare. In certain embodiments, patients with active lupus have SLEDAI score ≥6. A patient in a lupus pre-flare state may experience lupus flare in 1 day, 2 days, 3 days, 4 days, 5 days, 6 days, 1 week, 2 weeks, 3 weeks, in a month, or any time or range therebetween. A patient having active lupus may need further evaluation, consultation and/or treatment by a healthcare professional, such as a physician. The method can generate, recommend, and/or select based on the score and/or the inference, an action item, a method of evaluation, and/or a method of treatment for the patient. In certain embodiments, the method generate, recommend, and/or select the action item, the method of evaluation, and/or the method of treatment for the patient, based on the inference. In certain embodiments, a second machine learning model or a layer of the first machine learning model generate, recommend, and/or select the action item, the method of evaluation, and/or the method of treatment for the patient. The method of evaluation for the patient as generated, recommended and/or selected by the method may be a different method of evaluation for the patient than the method of evaluation for the patient being evaluated using the PRO data (e.g., if the score indicates a low effectiveness or high activity of the patient's disease/condition, e.g., patient having active lupus), or may be the same method of evaluation for the patient as the method of evaluation for the patient being evaluated using the PRO data (e.g., when the score indicates a high effectiveness of the current method of evaluation and/or treatment or a low activity of the patient's disease/condition, e.g., patient not having active lupus). The method of treatment for the patient as generated, recommended and/or selected by the method may be a different method of treatment for the patient than the method of treatment for the patient being evaluated using the PRO data (e.g., if the score indicates a low effectiveness or high activity of the patient's disease/condition, e.g., patient having active lupus), or may be the same method of treatment for the patient as the method of treatment for the patient being evaluated using the PRO data (e.g., when the score indicates a high effectiveness of the current method of evaluation and/or treatment or a low activity of the patient's disease/condition, e.g., patient not having the active lupus). In this manner, generating a new/different method of evaluation and/or treatment for the patient may be optional. In some embodiments, in the context of a drug or treatment clinical trial, the classification of the patient having active lupus or not having active lupus may indicate the level of effectiveness of the drug for the particular patient. For example, the classification of the patient having active lupus may indicate that the drug is not effective in treating the lupus in the patient. Conversely, the classification of the patient not having active lupus may indicate that the drug is effective in treating lupus in the patient. In certain embodiments, the action item, the method of evaluation, and/or the method of treatment for the patient, is generated, recommended, and/or selected based on a comparison of the score to a threshold score. In certain embodiments, for a patient classified as having the active lupus, the action item generated, recommended and/or selected, can include scheduling an appointment, visit, and/or consultation with a healthcare professional, such as a physician. The appointment, visit, and/or consultation can be between the healthcare professional, and the patient and/or a party responsible for the patient, and can be in-person, online, telephonic (e.g., telemedicine), and/or the like appointment, visit, and/or consultation. In certain embodiments, for a patient classified as having the active lupus, the action item generated, recommended and/or selected, can include sending the patient's classification, score and/or inference to a healthcare professional, such as a physician, with provisions for requirement of immediate attention.

For a patient classified as having the active lupus, the method of evaluation generated, recommended and/or selected can include a further test of lupus. The further test can include one or more tests, such as one or more genetic, blood, and/or laboratory tests, for determining type of the lupus such as endotype of lupus the patient has; severity of the lupus in the patient; and/or suitable treatment of lupus for the patient. In certain embodiments, the further test can include a genetic test, such as a test for determining endotype of lupus the patient has. In certain embodiments, the further test can include physician measures for calculating SLEDAI and/or PGA scores for the patient. The further test may be performed by a healthcare professional. For a patient classified as having the active lupus, the treatment (e.g., the treatment generated, recommended and/or selected by the method) can be a treatment for lupus. In certain embodiments, method includes performing the action item, performing the method of evaluation, and/or administering the treatment to the patient. In certain embodiments, a healthcare professional based on the score, inference, classification, and/or action item may generate, recommend, select, and/or perform the method of evaluation, and/or the method of treatment. In certain embodiments, the method includes performing the action item. In certain embodiments, method includes performing the method of evaluation. In certain embodiments, method includes administering the treatment (e.g., the treatment generated, recommended and/or selected by the method) to the patient. In certain embodiments, the treatment is administered based on the classification that the patient has active lupus. In certain embodiments, the treatment (e.g., the treatment generated, recommended and/or selected by the method) can include a pharmaceutical composition. The treatment for lupus can treat, reduce severity of, and/or reduce risk of having lupus, such as active lupus. In certain embodiments, the treatment for lupus comprises a neutrophil function inhibitor, a TNF inhibitor, an IL1 inhibitor, a Plasma cell inhibitor, a NK cell inhibitor, a B Cell Inhibitor, or any combination thereof. Non-limiting examples of an IFN inhibitor include Anifrolumab. Non-limiting examples of a Plasma cell inhibitor include Mycophenolate, Bortezomib, Carfilzomib, Ixazomib, Daratumumab, Isatuximab and Elotuzumab. Non-limiting examples of an IL1 inhibitor include Anakinra, and Canakinumab. Non-limiting examples of a TNF inhibitor include Adalimumab, Certolizumab pegol, Etanercept, Golimumab, and Infliximab. Non-limiting examples of a Neutrophil function inhibitor include Dasatinib, Apremilast, and Roflumilast. Non-limiting examples of a NK cell inhibitor include Azathioprine. Non-limiting examples of a B cell inhibitor include Belimumab, Rituximab, Obinutuzumab, and Inebilizumab. In certain embodiments, the treatment for lupus comprises Anifrolumab, Mycophenolate, Bortezomib, Carfilzomib, Ixazomib, Daratumumab, Isatuximab, Elotuzumab, Anakinra, Canakinumab Adalimumab, Certolizumab pegol, Etanercept, Golimumab, Infliximab, Dasatinib, Apremilast, Roflumilast, Azathioprine, Belimumab, Rituximab, Obinutuzumab, Inebilizumab, or any combination thereof. Lupus can be any type of lupus including but not limited to systemic lupus erythematosus (SLE), cutaneous lupus erythematosus, drug-induced lupus, and neonatal lupus. In certain embodiments lupus is SLE. In certain embodiments, the PRO data include one or more of SLAQ data, HRQOL data, Non-HRQOL data, Fatigue VAS data, Pain VAS data, PtGA data, FSS data, FACIT-F data, Morning Stiffness data, PROMIS-29 data, and SF-36 data. PRO data can include patient's response to the PRO questionnaires, for example FSS data includes patient's response to the FSS questionnaires. In certain embodiments, PROMIS-29 data can include Fatigue data, Sleep disturbance data, Depression data, Anxiety data, Pain Intensity data, Pain interference data, Satisfaction with social role data, physical function data, or any combination thereof. In certain embodiments, SF-36 data can include vitality, bodily pain, general health, mental health, physical function, role emotional, role physical, social function or any combination thereof. In certain embodiments, the PRO data comprises one or more of SLAQ data, HRQOL data, Non-HRQOL data, Fatigue VAS data, Pain VAS data, PtGA data, FSS data, FACIT-F data, Morning Stiffness data, Fatigue data, Sleep disturbance data, Depression data, Anxiety data, Pain Intensity data, Pain interference data, Satisfaction with social role data, physical function data, vitality data, bodily pain data, general health data, mental health data, physical function data, role emotional data, role physical data and social function data. In certain embodiments, the PRO data comprises SLAQ data, HRQOL data, Non-HRQOL data, Fatigue VAS data, Pain VAS data, PtGA data, FSS data, FACIT-F data, Morning Stiffness data, Fatigue data, Sleep disturbance data, Depression data, Anxiety data, Pain Intensity data, Pain interference data, Satisfaction with social role data, physical function data, vitality data, bodily pain data, general health data, mental health data, physical function data, role emotional data, role physical data and social function data. In certain embodiments, the PRO data consists of SLAQ data, HRQOL data, Non-HRQOL data, Fatigue VAS data, Pain VAS data, PtGA data, FSS data, FACIT-F data, Morning Stiffness data, Fatigue data, Sleep disturbance data, Depression data, Anxiety data, Pain Intensity data, Pain interference data, Satisfaction with social role data, physical function data, vitality data, bodily pain data, general health data, mental health data, physical function data, role emotional data, role physical data and social function data. Fatigue data, Sleep disturbance data, Depression data, Anxiety data, Pain Intensity data, Pain interference data, Satisfaction with social role data, and physical function data can be obtained from PROMIS-29 data. Vitality data, bodily pain data, general health data, mental health data, physical function data, role emotional data, role physical data and social function data can be obtained from SF-36 data. In certain embodiments, the PRO data comprise Fatigue VAS data, Pain VAS data, PtGA data, FSS data, FACIT-F data, Morning Stiffness data, or any combination thereof. In certain embodiments, the PRO data comprises one or more of PtGA data, Pain Intensity data (e.g., from PROMIS-29), mental health data (e.g., from SF-36), and social function data (e.g., from SF-36). In certain embodiments, the PRO data comprises at least PtGA data, Pain Intensity data, mental health data, and social function data. The Pain Intensity data can comprise patient responses to the Pain Intensity question(s) in the PROMIS-29 questionnaires. The mental health data can comprise patient responses to the mental health question(s) in the SF-36 questionnaires. The social function data can comprise patient responses to the social function question(s) in the SF-36 questionnaires. In certain embodiments, the PRO data comprises PtGA data, Pain Intensity data, mental health data, and social function data. In certain embodiments, the PRO data consists of PtGA data, Pain Intensity data, mental health data, and social function data. In certain embodiments, the PRO data comprises one or more of FACIT-F data, HRQOL data, Anxiety data (e.g., from PROMIS-29), and Bodily pain data (e.g., from SF-36). In certain embodiments, the PRO data comprises at least FACIT-F data, HRQOL data, Anxiety data, and Bodily pain data. The Anxiety data can comprise patient responses to the Anxiety question(s) in the PROMIS-29 questionnaires. The bodily pain data can comprise patient responses to the bodily pain question(s) in the SF-36 questionnaires. In certain embodiments, the PRO data comprises FACIT-F data, HRQOL data, Anxiety data, and Bodily pain data. In certain embodiments, the PRO data consists of FACIT-F data, HRQOL data, Anxiety data, and Bodily pain data. In certain embodiments, the patient has lupus. In certain embodiments, the patient is suspected of having lupus. In certain embodiments, the patient is asymptomatic of lupus. The first machine learning model can generate the score and/or inference using linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof. The algorithm of the first machine learning model can be a machine learning classifier mentioned in this paragraph. The machine learning classifier (e.g., linear regression, LOG, Ridge regression, Lasso regression, EN regression, SVM, GBM, kNN, GLM, NB classifier, neural network, a RF, deep learning algorithm, LDA, DTREE, ADB, CART, and/or hierarchical clustering) can be trained to obtain the first machine learning model. In some embodiments, the first machine learning model, is trained (e.g., obtained by training) using a supervised machine learning algorithm or an unsupervised machine learning algorithm, e.g., the classifier can be a supervised machine learning algorithm or an unsupervised machine learning algorithm. In certain embodiments, the first machine learning model generate the score and/or inference using linear regression. In certain embodiments, the first machine learning model generate the score and/or inference using LOG. In certain embodiments, the first machine learning model generate the score and/or inference using Ridge regression. In certain embodiments, the first machine learning model generate the score and/or inference using Lasso regression. In certain embodiments, the first machine learning model generate the score and/or inference using EN regression. In certain embodiments, the first machine learning model generate the score and/or inference using SVM. In certain embodiments, the first machine learning model generate the score and/or inference using GBM. In certain embodiments, the first machine learning model generate the score and/or inference using kNN. In certain embodiments, the first machine learning model generate the score and/or inference using GLM. In certain embodiments, the first machine learning model generate the score and/or inference using NB classifier. In certain embodiments, the first machine learning model generate the score and/or inference using neural network. In certain embodiments, the first machine learning model generate the score and/or inference using RF. In certain embodiments, the first machine learning model generate the score and/or inference using deep learning algorithm. In certain embodiments, the first machine learning model generate the score and/or inference using LDA. In certain embodiments, the first machine learning model generate the score and/or inference using DTREE. In certain embodiments, the first machine learning model generate the score and/or inference using ADB. In certain embodiments, the first machine learning model generate the score and/or inference using CART. In certain embodiments, the first machine learning model generate the score and/or inference using hierarchical clustering.

The first machine learning model can have a receiver operating characteristic (ROC) curve with an Area-Under-Curve (AUC) of at least about 0.7, at least about 0.75, at least about 0.8, at least about 0.85, at least about 0.9, at least about 0.91, at least about 0.92, at least about 0.93, at least about 0.94, at least about 0.95, at least about 0.96, at least about 0.97, at least about 0.98, at least about 0.99, or more than about 0.99. In certain embodiments, the first machine learning model has a ROC curve with an AUC of about 0.8 to about 1. In certain embodiments, the first machine learning model has a ROC curve with an AUC of about 0.8 to about 0.85, about 0.8 to about 0.9, about 0.8 to about 0.92, about 0.8 to about 0.93, about 0.8 to about 0.94, about 0.8 to about 0.95, about 0.8 to about 0.96, about 0.8 to about 0.97, about 0.8 to about 0.98, about 0.8 to about 0.99, about 0.8 to about 1, about 0.85 to about 0.9, about 0.85 to about 0.92, about 0.85 to about 0.93, about 0.85 to about 0.94, about 0.85 to about 0.95, about 0.85 to about 0.96, about 0.85 to about 0.97, about 0.85 to about 0.98, about 0.85 to about 0.99, about 0.85 to about 1, about 0.9 to about 0.92, about 0.9 to about 0.93, about 0.9 to about 0.94, about 0.9 to about 0.95, about 0.9 to about 0.96, about 0.9 to about 0.97, about 0.9 to about 0.98, about 0.9 to about 0.99, about 0.9 to about 1, about 0.92 to about 0.93, about 0.92 to about 0.94, about 0.92 to about 0.95, about 0.92 to about 0.96, about 0.92 to about 0.97, about 0.92 to about 0.98, about 0.92 to about 0.99, about 0.92 to about 1, about 0.93 to about 0.94, about 0.93 to about 0.95, about 0.93 to about 0.96, about 0.93 to about 0.97, about 0.93 to about 0.98, about 0.93 to about 0.99, about 0.93 to about 1, about 0.94 to about 0.95, about 0.94 to about 0.96, about 0.94 to about 0.97, about 0.94 to about 0.98, about 0.94 to about 0.99, about 0.94 to about 1, about 0.95 to about 0.96, about 0.95 to about 0.97, about 0.95 to about 0.98, about 0.95 to about 0.99, about 0.95 to about 1, about 0.96 to about 0.97, about 0.96 to about 0.98, about 0.96 to about 0.99, about 0.96 to about 1, about 0.97 to about 0.98, about 0.97 to about 0.99, about 0.97 to about 1, about 0.98 to about 0.99, about 0.98 to about 1, or about 0.99 to about 1. In certain embodiments, the first machine learning model has a ROC curve with an AUC of about 0.8, about 0.85, about 0.9, about 0.92, about 0.93, about 0.94, about 0.95, about 0.96, about 0.97, about 0.98, about 0.99, or about 1. In certain embodiments, the first machine learning model has a ROC curve with an AUC of at least about 0.8, about 0.85, about 0.9, about 0.92, about 0.93, about 0.94, about 0.95, about 0.96, about 0.97, about 0.98, or about 0.99. In certain embodiments, the first machine learning model has a ROC curve with an AUC of about 0.6 to about 1. In certain embodiments, the first machine learning model has a ROC curve with an AUC of about 0.6 to about 0.65, about 0.6 to about 0.7, about 0.6 to about 0.75, about 0.6 to about 0.8, about 0.6 to about 0.85, about 0.6 to about 0.9, about 0.6 to about 0.95, about 0.6 to about 1, about 0.65 to about 0.7, about 0.65 to about 0.75, about 0.65 to about 0.8, about 0.65 to about 0.85, about 0.65 to about 0.9, about 0.65 to about 0.95, about 0.65 to about 1, about 0.7 to about 0.75, about 0.7 to about 0.8, about 0.7 to about 0.85, about 0.7 to about 0.9, about 0.7 to about 0.95, about 0.7 to about 1, about 0.75 to about 0.8, about 0.75 to about 0.85, about 0.75 to about 0.9, about 0.75 to about 0.95, about 0.75 to about 1, about 0.8 to about 0.85, about 0.8 to about 0.9, about 0.8 to about 0.95, about 0.8 to about 1, about 0.85 to about 0.9, about 0.85 to about 0.95, about 0.85 to about 1, about 0.9 to about 0.95, about 0.9 to about 1, or about 0.95 to about 1. In certain embodiments, the first machine learning model has a ROC curve with an AUC of about 0.6, about 0.65, about 0.7, about 0.75, about 0.8, about 0.85, about 0.9, about 0.95, or about 1. In certain embodiments, the first machine learning model has a ROC curve with an AUC of at least about 0.6, about 0.65, about 0.7, about 0.75, about 0.8, about 0.85, about 0.9, or about 0.95.

The method can classify whether the patient has active lupus, or does not have active lupus with an accuracy of about 80% to about 100%. The method can classify whether the patient has active lupus, or does not have active lupus with an accuracy of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with an accuracy of about 80%, about 85%, about 90%, about 92%, about 93%, about 94%, about 95%, about 96%, about 97%, about 98%, about 99%, or about 100%. The method can classify whether the patient has active lupus, or does not have active lupus with an accuracy of at least about 80%, about 85%, about 90%, about 92%, about 93%, about 94%, about 95%, about 96%, about 97%, about 98%, or about 99%. In certain embodiments, the method classify whether the patient has active lupus, or does not have active lupus with an accuracy of about 60% to about 100%. In certain embodiments, the method classify whether the patient has active lupus, or does not have active lupus with an accuracy of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active lupus, or does not have active lupus with an accuracy of about 60%, about 65%, about 70%, about 75%, about 80%, about 85%, about 90%, about 95%, or about 100%. In certain embodiments, the method classify whether the patient has active lupus, or does not have active lupus with an accuracy of at least about 60%, about 65%, about 70%, about 75%, about 80%, about 85%, about 90%, or about 95%.

The method can classify whether the patient has active lupus, or does not have active lupus with a sensitivity of about 80% to about 100%. The method can classify whether the patient has active lupus, or does not have active lupus with a sensitivity of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with a sensitivity of about 80%, about 85%, about 90%, about 92%, about 93%, about 94%, about 95%, about 96%, about 97%, about 98%, about 99%, or about 100%. The method can classify whether the patient has active lupus, or does not have active lupus with a sensitivity of at least about 80%, about 85%, about 90%, about 92%, about 93%, about 94%, about 95%, about 96%, about 97%, about 98%, or about 99%. In certain embodiments, the method classify whether the patient has active lupus, or does not have active lupus with a sensitivity of about 60% to about 100%. In certain embodiments, the method classify whether the patient has active lupus, or does not have active lupus with a sensitivity of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active lupus, or does not have active lupus with a sensitivity of about 60%, about 65%, about 70%, about 75%, about 80%, about 85%, about 90%, about 95%, or about 100%. In certain embodiments, the method classify whether the patient has active lupus, or does not have active lupus with a sensitivity of at least about 60%, about 65%, about 70%, about 75%, about 80%, about 85%, about 90%, or about 95%.

The method can classify whether the patient has active lupus, or does not have active lupus with a specificity of about 80% to about 100%. The method can classify whether the patient has active lupus, or does not have active lupus with a specificity of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with a specificity of about 80%, about 85%, about 90%, about 92%, about 93%, about 94%, about 95%, about 96%, about 97%, about 98%, about 99%, or about 100%. The method can classify whether the patient has active lupus, or does not have active lupus with a specificity of at least about 80%, about 85%, about 90%, about 92%, about 93%, about 94%, about 95%, about 96%, about 97%, about 98%, or about 99%. In certain embodiments, the method classify whether the patient has active lupus, or does not have active lupus with a specificity of about 60% to about 100%. In certain embodiments, the method classify whether the patient has active lupus, or does not have active lupus with a specificity of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active lupus, or does not have active lupus with a specificity of about 60%, about 65%, about 70%, about 75%, about 80%, about 85%, about 90%, about 95%, or about 100%. In certain embodiments, the method classify whether the patient has active lupus, or does not have active lupus with a specificity of at least about 60%, about 65%, about 70%, about 75%, about 80%, about 85%, about 90%, or about 95%.

The method can classify whether the patient has active lupus, or does not have active lupus with a positive predictive value of about 80% to about 100%. The method can classify whether the patient has active lupus, or does not have active lupus with a positive predictive value of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with a positive predictive value of about 80%, about 85%, about 90%, about 92%, about 93%, about 94%, about 95%, about 96%, about 97%, about 98%, about 99%, or about 100%. The method can classify whether the patient has active lupus, or does not have active lupus with a positive predictive value of at least about 80%, about 85%, about 90%, about 92%, about 93%, about 94%, about 95%, about 96%, about 97%, about 98%, or about 99%. In certain embodiments, the method classify whether the patient has active lupus, or does not have active lupus with a positive predictive value of about 60% to about 100%. In certain embodiments, the method classify whether the patient has active lupus, or does not have active lupus with a positive predictive value of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active lupus, or does not have active lupus with a positive predictive value of about 60%, about 65%, about 70%, about 75%, about 80%, about 85%, about 90%, about 95%, or about 100%. In certain embodiments, the method classify whether the patient has active lupus, or does not have active lupus with a positive predictive value of at least about 60%, about 65%, about 70%, about 75%, about 80%, about 85%, about 90%, or about 95%.

The method can classify whether the patient has active lupus, or does not have active lupus with a negative predictive value of about 80% to about 100%. The method can classify whether the patient has active lupus, or does not have active lupus with a negative predictive value of about 80% to about 85%, about 80% to about 90%, about 80% to about 92%, about 80% to about 93%, about 80% to about 94%, about 80% to about 95%, about 80% to about 96%, about 80% to about 97%, about 80% to about 98%, about 80% to about 99%, about 80% to about 100%, about 85% to about 90%, about 85% to about 92%, about 85% to about 93%, about 85% to about 94%, about 85% to about 95%, about 85% to about 96%, about 85% to about 97%, about 85% to about 98%, about 85% to about 99%, about 85% to about 100%, about 90% to about 92%, about 90% to about 93%, about 90% to about 94%, about 90% to about 95%, about 90% to about 96%, about 90% to about 97%, about 90% to about 98%, about 90% to about 99%, about 90% to about 100%, about 92% to about 93%, about 92% to about 94%, about 92% to about 95%, about 92% to about 96%, about 92% to about 97%, about 92% to about 98%, about 92% to about 99%, about 92% to about 100%, about 93% to about 94%, about 93% to about 95%, about 93% to about 96%, about 93% to about 97%, about 93% to about 98%, about 93% to about 99%, about 93% to about 100%, about 94% to about 95%, about 94% to about 96%, about 94% to about 97%, about 94% to about 98%, about 94% to about 99%, about 94% to about 100%, about 95% to about 96%, about 95% to about 97%, about 95% to about 98%, about 95% to about 99%, about 95% to about 100%, about 96% to about 97%, about 96% to about 98%, about 96% to about 99%, about 96% to about 100%, about 97% to about 98%, about 97% to about 99%, about 97% to about 100%, about 98% to about 99%, about 98% to about 100%, or about 99% to about 100%. The method can classify whether the patient has active lupus, or does not have active lupus with a negative predictive value of about 80%, about 85%, about 90%, about 92%, about 93%, about 94%, about 95%, about 96%, about 97%, about 98%, about 99%, or about 100%. The method can classify whether the patient has active lupus, or does not have active lupus with a negative predictive value of at least about 80%, about 85%, about 90%, about 92%, about 93%, about 94%, about 95%, about 96%, about 97%, about 98%, or about 99%. In certain embodiments, the method classify whether the patient has active lupus, or does not have active lupus with a negative predictive value of about 60% to about 100%. In certain embodiments, the method classify whether the patient has active lupus, or does not have active lupus with a negative predictive value of about 60% to about 65%, about 60% to about 70%, about 60% to about 75%, about 60% to about 80%, about 60% to about 85%, about 60% to about 90%, about 60% to about 95%, about 60% to about 100%, about 65% to about 70%, about 65% to about 75%, about 65% to about 80%, about 65% to about 85%, about 65% to about 90%, about 65% to about 95%, about 65% to about 100%, about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 100%, about 85% to about 90%, about 85% to about 95%, about 85% to about 100%, about 90% to about 95%, about 90% to about 100%, or about 95% to about 100%. In certain embodiments, the method classify whether the patient has active lupus, or does not have active lupus with a negative predictive value of about 60%, about 65%, about 70%, about 75%, about 80%, about 85%, about 90%, about 95%, or about 100%. In certain embodiments, the method classify whether the patient has active lupus, or does not have active lupus with a negative predictive value of at least about 60%, about 65%, about 70%, about 75%, about 80%, about 85%, about 90%, or about 95%.

In some embodiments, to validate PRO data, a device may verify the equivalence of paper and electronic administration methods for patient surveys. ICC values may be used to verify that the equivalence of paper and electronic administration methods for patient surveys improves as more surveys are taken and provided. Bland-Altman plots may be used to identify any bias between paper and ePRO data. To account for any discrepancies between paper and ePRO data, a machine learning model may adjust weights used to evaluate the paper and ePRO data. For example, a higher weight may be used for data with higher reliability.

In some embodiments, a machine learning model used to generate predicted health scores may be trained to generate the scores so that no adjustment is needed to a current method of treatment a certain percentage of the time (e.g., 80% or some other number). For example, when the health score indicates that a method of treatment should be changed (e.g., the score is above a threshold that indicates severity of a condition or patient's reactions), the machine learning model may adjust the method of treatment. Therefore, the machine learning model may be trained to generate a score that is below the threshold value a certain percentage of the time. For example, if a Physician's Global Assessment Score indicates that 80% of the time the physician would not change the patient's current method of treatment, the training data may train the machine learning model to generate a score that would satisfy the threshold 80% of the time. In this manner, the input data to the machine learning model would result in a score that does not satisfy the threshold 20% of the time. To achieve the adjustment, the machine learning model may adjust the weight of certain input data up or down. For example, to increase the rate at which the score results in a change in treatment, the weights of certain data may be decreased to produce a lower score (or increased to produce a higher score, depending on the thresholds used). In this manner, one patient's data may result in a change in treatment, while another patient's data may not result in a change in treatment, based on the evaluation criteria used by the machine learning model to generate and evaluate a score.

In some embodiments, the correlation between patient reporting modes (e.g., ePRO and paper modes) may indicate a strong or weak relationship between the reporting modes. When the correlation (e.g., ICC) exceeds a threshold value indicative of a strong relationship between the reporting modes for any particular reported data (e.g., physical data, mental data, pain level, etc.), the weight of the reported data, as used by the machine learning model, may be higher for the reported data. In this manner, the machine learning model may adjust its weights based on the correlations reliability of the reported data. Data that is more reliable may be weighted higher than data that is less reliable (e.g., based on the relationship between user-reported modes and a healthcare professional-reported modes).

In some embodiments, the PRO and/or physician data of any patients may be compared to identify patient similarities. For example, a correlation between patients may allow for machine learning to determine whether a patient would respond to a treatment or evaluation plan based on whether other patients with similar data have responded to the treatment or evaluation plan. In this manner, by having a large data set of patient data for multiple patients, the most informative types of data may be used to generate predictive models (e.g., machine learning) to predict how a patient may react to a treatment or evaluation plan, and therefore whether to adjust/select a treatment or evaluation plan for a patient.

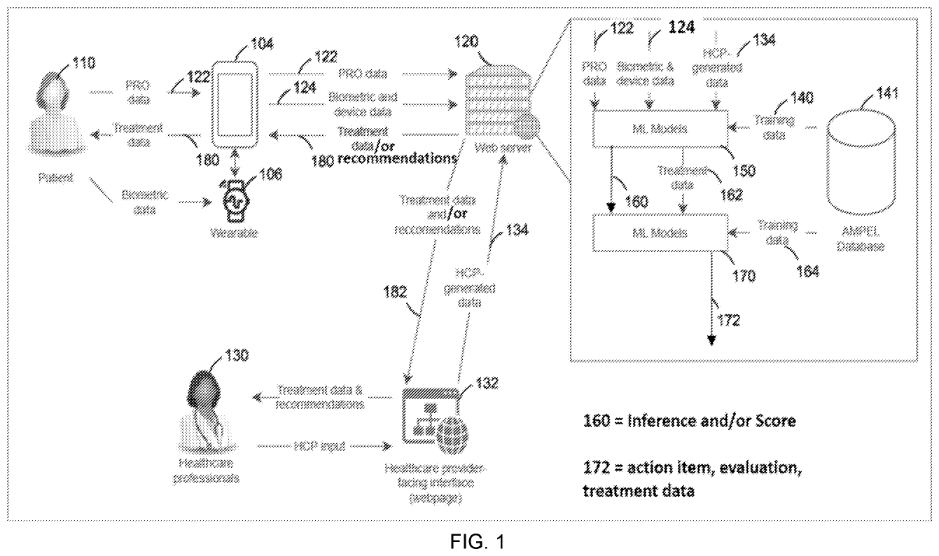

FIG. 1 shows a non-limiting example of a system 100 for analyzing patient-reported outcome (PRO) data according to embodiments described herein.

Referring to FIG. 1, the system 100 may include devices, such as device 104 and device 106, may have one or more executable applications used by one or more patients 110. The devices may be used to provide data (e.g., via the one or more executable applications) to one or more remote devices 120 (e.g., servers, cloud-based devices, etc.). For example, the device 104 may collect PRO data 122 from the one or more patients 110 based on user inputs received during a time period (e.g., hourly data, daily data, weekly data, etc.). The PRO data 122 may represent the perceptions and reactions of the one or more patients 110 to patient's health, a disease evaluation method, and/or a treatment plan (e.g., including treatment using pharmaceutical and/or biologics products). The device 106 may optionally provide a combination of biometric and/or device data 124 (e.g., biometric data including sleep data, breathing data, body temperature data, heart rate data, etc., and device motion data including accelerometer or other data indicative of activity, steps, etc.) to the one or more remote devices 120 for analysis. One or more healthcare professionals 130 may, using devices 132, optionally provide HCP-generated data 134 indicative of observations of the one or more patients 110 provided by the one or more healthcare professionals 130.

Still referring to FIG. 1, the one or more remote devices 120 may input the PRO data 122, the optional biometric and/or device data 124, and opttional the HCP-generated data 134, along with optional training data 140 (e.g., stored in a database 141), to one or more machine learning models 150, which may generate one or more health scores, and/or inference 160 indicative of the activity of a patient's condition/disease and/or the effectiveness of a current treatment and/or severity of the patient's reactions. The health scores 160 may include a Physician's Global Assessment Score, a Systematic Lupus Erythematosus Disease Activity Index (SLEDAI) Score, or other scores predicting how a health professional would score a patient based on the PRO data 122 and/or the optional biometric and/or device data 124. The inference 160 can be whether the input data, e.g., PRO data 122 is indicative to the patient having active or inactive disease. Based on score (e.g., health score) and/or inference 160, an action item, evaluation and/or treatment 172 can be generated, recommended and/or selected. In certain embodiments, the one or more health scores and/or inference 160 may be input into one or more machine learning models 170 (e.g., different from or different layers of the one or more machine learning models 150) optionally along with patient treatment data 162 (e.g., indicating current treatment plans of the one or more patients 110, and the responses of the one or more patients 110 to treatment plans given the PRO data 122 and the biometric and/or device data 124), and optional training data 164 (e.g., stored in the database 141), and the one or more machine learning models 170 may generate the action item, evaluation and/or treatment 172. The treatment 172 which may be to maintain the current patient treatments, or may represent a modified treatment (e.g., based on whether the scores and/or inference 160 indicate that a current treatment or evaluation plan is ineffective and/or based on whether patients with similar data have reacted positively to another treatment or evaluation plan). In some embodiments, the action item, 172, based on the score and/or inference can be scheduling an appointment, visit, and/or consultation for the patient with a healthcare professional, such as a physician. In some embodiments, the action item, 172, based on the score and/or inference, can be sending the patient's score and/or inference to a healthcare professional, such as a physician. In some embodiments, the evaluation, 172 e.g., for a patient classified as having active disease, can be further tests for the device. In certain embodiments, the one or more remote devices 120 may send treatment data and/or recommendation (e.g., action item and/or evaluation) 180 to the devices 102 and may send treatment data and/or recommendation 182 to the one or more devices 132 for presentation and/or action (e.g., to indicate 172).

In some embodiments, the one or more executable applications may be used to collect the PRO 122 data in an electronic format (e.g., ePRO). The one or more executable applications may receive patient inputs that indicate the patient's perception of their own health. In addition, the devices 104 and/or 106 may collect biometric data (e.g., sleep data, breathing data, body temperature data, heart rate data, etc.), device motion data (e.g., indicative of activity, steps, etc.), and/or geo-location data from patients.

In some embodiments, the one or more remote devices 120 may determine, generate recommend, and/or select an action a method of evaluation and treatment of patients, which may include receiving the PRO data 122 from the devices 102, the PRO data 122 including ePRO data and/or paper-administered data, indicating patient reactions to their health, and/or patient reactions to a method of evaluation and/or treatment using a pharmaceutical or biological treatment. The one or more remote devices 120 may validate the PRO data 122 and/or the HCP-generated data 134 according to regulatory requirements.

In some embodiments, to validate PRO data 122 and/or the HCP-generated data 134, the one or more remote devices 120 may verify the equivalence of paper and electronic administration methods for patient surveys. ICC values may be used to verify that the equivalence of paper and electronic administration methods for patient surveys improves as more surveys are taken and provided. Bland-Altman plots may be used to identify any bias between paper and ePRO data. To account for any discrepancies between paper and ePRO data, the one or more machine learning models 150 may adjust weights used to evaluate the HCP-generated data 134 and/or the ePRO data 122. For example, a higher weight may be used for data with higher reliability.

In some embodiments, the one or more machine learning models 150 used to generate predicted health scores 160 and may be trained to generate the scores so that no adjustment is needed to a current method of treatment a certain percentage of the time (e.g., 80% or some other number). For example, when the health score 160 indicates that a method of treatment should be changed (e.g., the score is above a threshold that indicates severity of a condition or patient's reactions), the one or more machine learning models 170 may adjust the method of treatment. Therefore, the one or more machine learning models 150 may be trained to generate a score that is below the threshold value a certain percentage of the time. For example, if a Physician's Global Assessment Score indicates that 80% of the time the physician would not change the patient's current method of treatment, the training data may train the machine learning model to generate a score that would satisfy the threshold 80% of the time. In this manner, the input data to the machine learning model would result in a score that does not satisfy the threshold 20% of the time. To achieve the adjustment, the one or more machine learning models 150 may adjust the weight of certain input data up or down. For example, to increase the rate at which the score results in a change in treatment, the weights of certain data may be decreased to produce a lower score (or increased to produce a higher score, depending on the thresholds used). In this manner, one patient's data may result in a change in treatment, while another patient's data may not result in a change in treatment, based on the evaluation criteria used by the one or more machine learning models 150 and 170 to generate and evaluate a score.

In some embodiments, the correlation between patient reporting modes (e.g., the PRO data 122 and the HCP-generated data 134) may indicate a strong or weak relationship between the reporting modes. When the correlation (e.g., ICC) exceeds a threshold value indicative of a strong relationship between the reporting modes for any particular reported data (e.g., physical data, mental data, pain level, etc.), the weight of the reported data, as used by the one or more machine learning models 150, may be higher for the reported data. In this manner, the one or more machine learning models 150 may adjust weights based on the correlations reliability of the reported data. Data that is more reliable may be weighted higher than data that is less reliable (e.g., based on the relationship between user-reported modes and a healthcare professional-reported modes).

In some embodiments, the one or more remote devices 120 may have access to patient profile data (in accordance with relevant laws and with patient consent). The data may be mined by the one or more remote devices 120 to select the most informative features (e.g., pain, fatigue, mental health, etc.) to develop predictive algorithms used by the one or more machine learning models 150 and/or 170. The one or more machine learning models 150 and/or 170 may use a Random Forest or other method to select features that may be most likely to contribute to a health score (e.g., Physician's Global Assessment Score, SLEDAI score, etc.) and/or inference 160. The one or more machine learning models 150 and/or 170 may predict one or more health scores that a physician may generate based on the PRO data (e.g., a prediction of how the physician would score the patient if presented the same data as the patient provided), and/or the inference. The one or more machine learning models 150 and/or 170 may predict whether a patient will respond to a treatment or evaluation based on available patient data. In this manner, the treatment or evaluation of a patient may be adjusted when the patient's data are similar to data of other patients who responded to a treatment or evaluation. In this manner, the correlation between physician data and PRO data may be used to predict a physician's health score, and the correlation between PRO and/or physician data of patients may be used to predict a patient's reactiveness to a treatment or evaluation plan.

In some embodiments, the one or more machine learning models 150 and/or 170 may classify patients as “active” or “inactive” based on the PRO data 122 and optionally the biometric and/or device data 124 (e.g., location data, etc.). For example, an active patient, such as an active lupus patient may have a SLEDAI score of ≥6, and inactive patient, such as an inactive lupus patient may have a SLEDAI score of <6. Patients with active SLE may experience a variety of clinical manifestations, including flares of varying severity. A determination of whether a patient is active or inactive may be based on generalized linear models, k-nearest neighbors, and/or random forest. The biometric and/or device data 124 may be used to supplement the PRO data 122 to determine whether a patient's disease/condition is active or inactive. For example, if the biometric and/or device data 124 is consistent with data classified as active or inactive, the one or more machine learning models 150 and/or 170 may consider the biometric and/or device data 124 as an indicator of an active or inactive patient.

In some embodiments, the one or more machine learning models 150 and/or 170 may adjust criteria by which the one or more health scores 160 and/or the one or more treatment recommendations 172 are generated. For example, when the one or more health scores 160 result in a change in treatment plan more than a threshold percentage of time, the criteria may be adjusted (e.g., some types of patient data may be weighted higher or lower). In another example, the criteria used to determine the treatment recommendations 172 may be adjusted based on data that shows that physicians adjust patient treatment plans of patient's whose data is similar to the PRO data 122 and the biometric and/or device data 124.

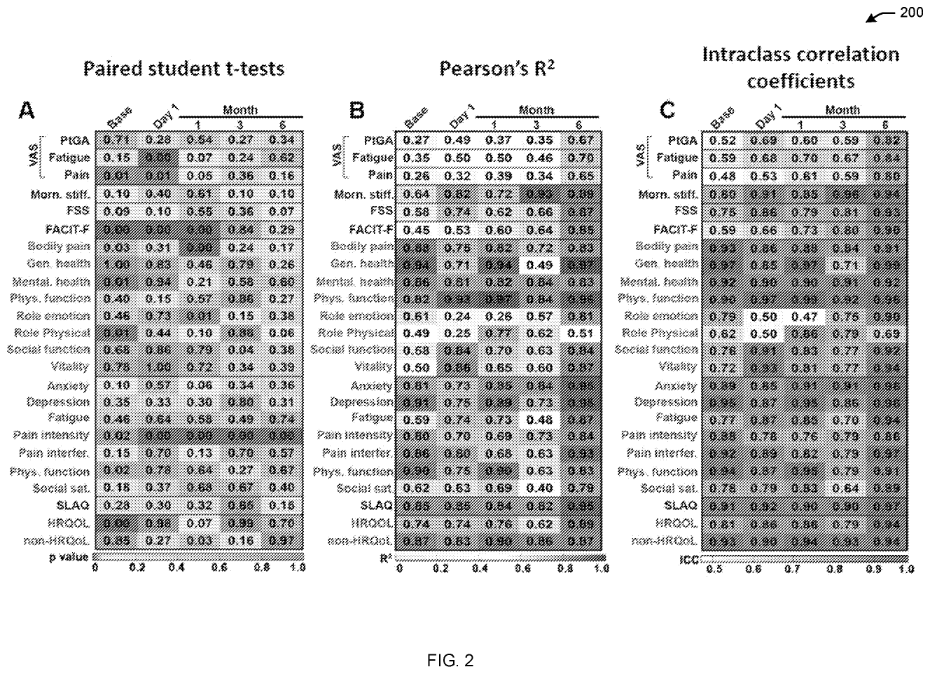

FIG. 2 shows a non-limiting example of application-based PRO data collection 200 using the systems and methods described herein.

Referring to FIG. 2, the application-based PRO data collection 200 may include a variety of data collection methods, such as paired t-tests (example in left panel), Pearson's R² (coefficients of determination, example in center panel), and ICC (example in right panel), and may allow for a variety of patient data, such as fatigue, pain, mental health, physical function, pain intensity, and other data shown. The data shown covers a time period that begins with a base time, a first date, and multiple subsequent months (e.g., 1, 3, 6, etc.). The data and time periods shown are meant to be examples and not exclusive. The data shown may be used to measure the strength of relationships/differences between information collected using different administration methods. As shown, some data is more reliable than other data. Accordingly, the one or more machine learning models 150 of FIG. 1 may assume the more reliable data higher weights than the less reliable data for determining the one or more health scores 160. The one or more machine learning models 150 and/or 170 may evaluate the HCP-generated data 134 to determine the one or more health scores 160 that physicians would generate based on the patient data (e.g., physical, mental, pain level, etc.), and based on any correlation between PRO and physician data that may indicate that some patient data responses are more reliable than others.

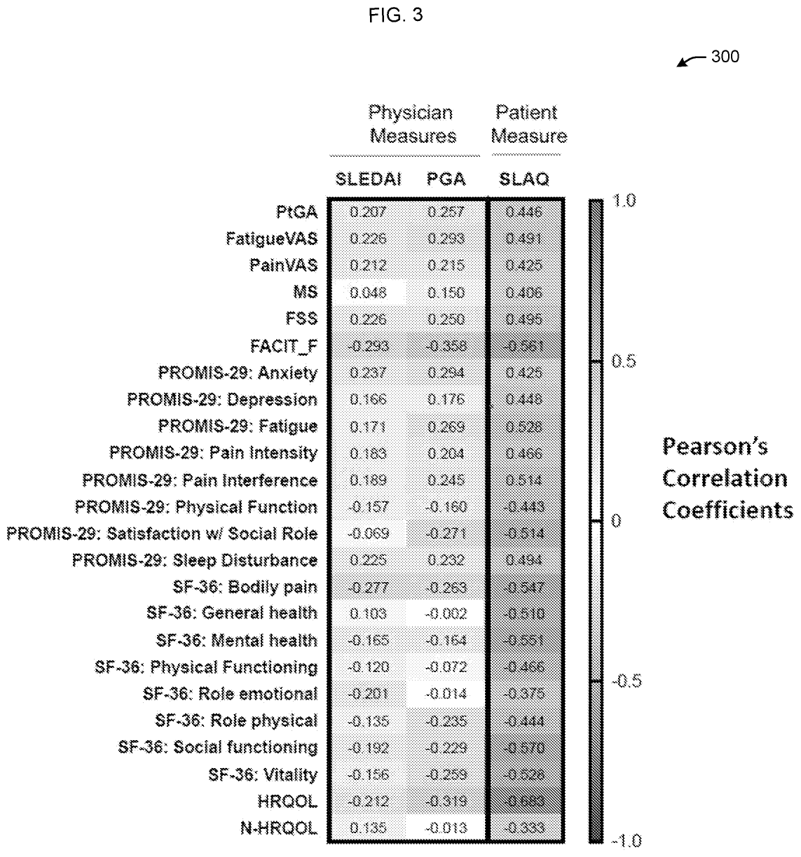

FIG. 3 shows a non-limiting example of PRO data 300 compared to physician measures using the systems and methods described herein.

Referring to FIG. 3, the PRO data 300 may include physician measures (e.g., SLEDAI, PGA, etc.) and patient measures (e.g., systematic lupus activity questionnaire-SLAQ), and Pearson's correlation coefficients for the PRO data 300. Using the PRO data 300, the one or more machine learning models 150 of FIG. 1 may evaluate the relationship between ePRO survey scores and clinician-reported data that evaluates patients. FIG. 3 shows that the correlation between physician evaluations and individual PROs may be weak.

However, the systems and methods presented herein provide an enhancement in that the PROs and machine learning models, the aggregate of PRO information may be able to classify patients as “active” or “inactive” according to the physician's evaluation. By establishing the relation between remote ePROs and clinician-reported data, the systems and methods presented herein may use ePROs to track patients' responses to treatment.

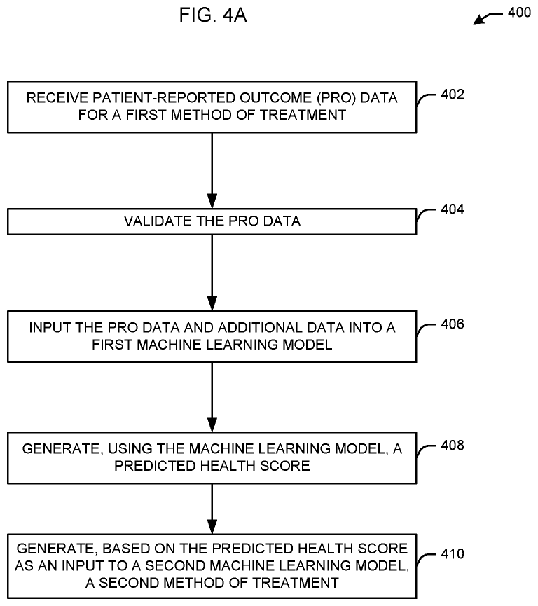

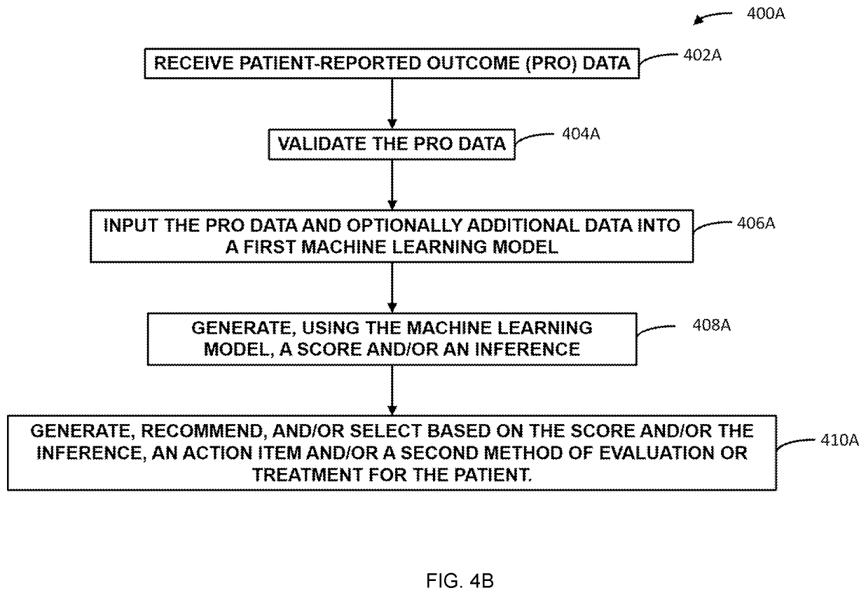

FIGS. 4A and B show non-limiting examples of work flow 400 and 400A respectively for analyzing patient-reported outcome (PRO) data according to embodiments described herein.

Referring FIG. 4A, at block 402, a device (or system, e.g., the one or more remote devices 120 of FIG. 1) may receive PRO data for one or more patients (e.g., the one or more patients 110 of FIG. 1) and additional data, such as biometric and/or device data (e.g., the biometric and/or device data 124 of FIG. 1), paper data (e.g., the HCP-generated data 134 of FIG. 1, such as physician-provided data for patients), treatment data (e.g., the treatment data 162 of FIG. 1) indicating current and past treatments of patients, and the like.

At block 404, the device may validate the PRO data and any other received data. To validate PRO data, the device may verify the equivalence of paper and electronic administration methods for patient surveys. ICC values may be used to verify that the equivalence of paper and electronic administration methods for patient surveys improves as more surveys are taken and provided. Bland-Altman plots may be used to identify any bias between paper and ePRO data. To account for any discrepancies between paper and ePRO data, a machine learning model may adjust weights used to evaluate the paper and ePRO data. For example, a higher weight may be used for data with higher reliability.

At block 406, the device may input the PRO data and any additional data, such as biometric and/or device data, the paper data, and the like, to a first machine learning model (e.g., the one or more machine learning models 150 of FIG. 1). The machine learning model may include a neural network, a deep learning algorithm, a support vector machine, a random forest, a similarity network, a difference network, or another type of machine learning model that may be trained or untrained, and that may analyze the data inputs.

At block 408, the device may generate, using the machine learning model, a predicted health score. The predicted health score may represent a predicted score that would otherwise be generated by a healthcare professional given the data inputs. The predicted health score may represent a Physician's Global Assessment Score, a SLEDAI score, or another score, and may be indicative of a patient's reactions to a disease/condition and/or the effectiveness of a patient's current treatment plan (e.g., using a pharmaceutical or biologics product). For example, the PRO and/or other data may be used to generate a physician's assessment score in an automated fashion, such as by using machine learning that inputs the PRO and/or biometric data, including ePRO and paper-administered PRO data, and generates a score that estimates what a physician would generate if a physician were analyzing the patient's data.

At block 410, the device may generate, based on the predicted health score as an input to a second machine learning model (e.g., the one or more machine learning models 170 of FIG. 1), along with patient treatment data as inputs to the second machine learning model, a method of treatment for a patient that may be the same as or different than the current method of treatment for the patient. The method of treatment for the patient as generated by the second machine learning model may be a different method of evaluation and/or treatment for the patient than the current method of evaluation and/or treatment (e.g., if the score indicates a low effectiveness or high activity of the patient's disease/condition), or may be the same method treatment for the patient as the method of evaluation and/or treatment for the patient being evaluated using the PRO data (e.g., when the score indicates a high effectiveness of the current method of evaluation and/or treatment or a low activity of the patient's disease/condition).

Referring FIG. 4B, at block 402A, a device (or system, e.g., the one or more remote devices 120 of FIG. 1) may receive PRO data for one or more patients (e.g., the one or more patients 110 of FIG. 1) and optionally additional data, such as biometric and/or device data (e.g., the biometric and/or device data 124 of FIG. 1), doctor's data (e.g., the HCP-generated data 134 of FIG. 1, such as physician-provided data for patients), treatment data (e.g., the treatment data 162 of FIG. 1) indicating current and past treatments of patients, and the like. The PRO data (122 of FIG. 1) can include patient(s)' reactions to their health, and/or a first method of evaluation or treatment using a pharmaceutical or biological treatment.

At block 404A, the device may validate the PRO data and any other received data. To validate PRO data, the device may verify the equivalence of paper and electronic administration methods for patient surveys. ICC values may be used to verify that the equivalence of paper and electronic administration methods for patient surveys improves as more surveys are taken and provided. Bland-Altman plots may be used to identify any bias between paper and ePRO data. To account for any discrepancies between paper and ePRO data, a machine learning model may adjust weights used to evaluate the paper and ePRO data. For example, a higher weight may be used for data with higher reliability.

At block 406A, the device may input the PRO data and optionally the additional data, such as biometric and/or device data, the doctor's data, and the like, to a first machine learning model (e.g., the one or more machine learning models 150 of FIG. 1). The machine learning model may include a neural network, a deep learning algorithm, a support vector machine, a random forest, a similarity network, a difference network, or another type of machine learning model that may be trained or untrained, and that may analyze the data inputs.

At block 408A, the device may generate, using the machine learning model, a score (e.g. health score) and/or an inference. The score may represent a predicted score that would otherwise be generated by a healthcare professional given the data inputs. The score may represent a Physician's Global Assessment Score, a SLEDAI score, or another score, and may be indicative of a patient's reactions to a disease/condition and/or the effectiveness of a patient's current treatment plan (e.g., using a pharmaceutical or biologics product). For example, the PRO and/or other data may be used to generate a physician's assessment score in an automated fashion, such as by using machine learning that inputs the PRO and/or biometric data, including ePRO and paper-administered PRO data, and generates a score that estimates what a physician would generate if a physician were analyzing the patient's data. The score and/or inference can be indicative of the disease state of the patient. The disease state of the patient can be the patient having the active disease, or not having active disease. The inference generated can be whether the input data, such as ePRO data, is indicative of the patient having active disease, or not having active disease. In certain embodiments, the score can be indicative of the patient having active disease or not having active disease. The method can classify a patient as having active disease, or not having active disease, based on the score and/or inference. The inference and the score can be a classification parameter, and the method can classify the patient based on the classification parameter.

At block 410A, the device may generate, recommend, and/or select based on the score and/or the inference, an action item and/or a second method of evaluation or treatment for the patient. In certain embodiments, the action item, e.g., for a patient classified as having active disease, can be scheduling an appointment, visit, and/or consultation for the patient with a healthcare professional, such as a physician. The appointment, visit, and/or consultation can be between the healthcare professional, and the patient and/or a party responsible for the patient, and can be in-person, online, telephonic (e.g., telemedicine), and/or the like appointment, visit, and/or consultation. In certain embodiments, the action item, e.g., for a patient classified as having active disease, can include sending the patient's classification, score and/or inference to a healthcare professional, such as a physician, with provisions for requirement of immediate attention. In certain embodiments, the evaluation can be further test of the disease. The method of treatment for a patient that may be the same as or different than the current method of treatment for the patient. The method of treatment for the patient as generated by the second machine learning model may be a different method of evaluation and/or treatment for the patient than the current method of evaluation and/or treatment (e.g., if the score indicates a low effectiveness or high activity of the patient's disease/condition), or may be the same method treatment for the patient as the method of evaluation and/or treatment for the patient being evaluated using the PRO data (e.g., when the score indicates a high effectiveness of the current method of evaluation and/or treatment or a low activity of the patient's disease/condition).

FIG. 5 shows a non-limiting example of a machine learning work flow 500 for analyzing PRO data according to embodiments described herein.

Referring to FIG. 5, a primary/principal component analysis may receive, as input data, PRO data (e.g., the PRO data 122 of FIG. 1) and biometric and device data (e.g., the biometric and/or device data 124 of FIG. 1). The outputs of the primary/principal component analysis may be inputs to a features selection component. The input data may be mined by the primary/principal component analysis to select the most informative features (e.g., pain, fatigue, mental health, etc.) to develop predictive algorithms that allow a feature selection component to select features that may be most likely to contribute to a health score (e.g., Physician's Global Assessment Score, SLEDAI score, etc.). Based on the features selected by the feature selection component, a classification model may classify patients as active or inactive, and may generate an assessment of the patient disease activity (e.g., a health score). Training data, such as PRO data and HCP-generated data, may be input to train an unsupervised clustering model. The output generated by the unsupervised clustering model may be input to a cluster validation and statistical analysis model, which may receive input data from HCPs. The output of the cluster validation and statistical analysis model may be labeled training data that may be input to the classification model to generate patient classifiers (e.g., active and inactive).

FIGS. 6A-H show non-limiting examples of unsupervised clustering 600A-H for analyzing PRO data according to embodiments described herein.

The unsupervised clustering 600A-H may receive as inputs PRO and optionally biometric data, such as steps (e.g., walked/ran) data, sleep data, distance traveled data, Patient Global Assessment (PtGA) data, pain visual analogue scale (pain VAS) data, fatigue VAS data, morning stiffness data, functional status scale (FSS) data, functional assessment of chronic illness therapy-fatigue (FACIT-F) data, short-form (SF-36) data, pain intensity (PROMIS-29) data, health-related quality of life (HRQOL) data, non-HRQOL (NHRQOL) data, the Systemic Lupus Activity Questionnaire (SLAQ) and the like. PROMIS-29 data can include Fatigue data, Sleep disturbance data, Depression data, Anxiety data, Pain Intensity data, Pain interference data, Satisfaction with social role data, physical function data, or any combination thereof. SF-36 data can include vitality, bodily pain, general health, mental health, physical function, role emotional, role physical, social function or any combination thereof. For example, the unsupervised clustering may be performed by the unsupervised clustering model of FIG. 5. The PRO questionnaires and measurement tools, such as PtGA, pain VAS, fatigue VAS are described in Thanou A, et al. Lupus Sei Med 2019;6: e000365; morning stiffness is described in Alten R, et al. Scand.J Rheumatol 2015:44:354-58; FSS is described in Arthritis Rheum 2007;57:1348-57;FACIT-F is described in Kosinski M, et al. Lupus 2013:22:422-30; SF-36 is described in Stoll T. et al. J Rheumatol 1997,24:1608-14; PROMIS-29 is described in Katz P, et, al. Arthritis Care Res (Hoboken) 2017;69: 1312-21; HRQOL, and non-HRQOL are described in Jolly M, et al. Semin Arthrins Rheum 2012:42:56-65; and SLAQ is described in Romero-Diaz J, et al. Arthritis Care Res (Hoboken) 2011;63; each of which are incorporated in full herein by reference, and in Example 1.

Referring to FIG. 6A, in a non-limiting example ePRO data 122 and biometric data 124 from patients 110 including steps (e.g., walked/ran) data, sleep data, distance traveled data, Patient Global Assessment (PtGA) data, pain visual analogue scale (pain VAS) data, fatigue VAS data, morning stiffness data, functional status scale (FSS) data, functional assessment of chronic illness therapy-fatigue (FACIT-F) data, short-form (SF-36) data, pain intensity (PROMIS-29) data, health-related quality of life (HRQOL) data, non-HRQOL (NHRQOL) data was used as inputs for unsupervised clustering of the patients. As shown in FIG. 6A, using the input data patients were clustered into two clusters cluster 1 and cluster 2, by unsupervised clustering and primary/principal component analysis. Further, as shown in FIG. 6A, patients in cluster 2, have high SLEDAI score and are patients with active lupus, and patients in cluster 1, have low high SLEDAI score and are patients without active lupus.

Referring to FIGS. 6B-E in another non-limiting example, the electronic PRO (ePRO) data 122 of patients 110 meeting the American College of Rheumatology (ACR) definition of SLE was collected over a period of 6 months as part of a multicenter clinical trial (NCT03098823). A smartphone application was developed to collect 10 separate PROs according to a schedule, collecting over 70,000 total records. The PROs included SLAQ, HRQOL, Non-HRQOL, Fatigue VAS, Pain VAS, PtGA, FSS, FACIT-F, Morning Stiffness, Fatigue, Sleep disturbance, Depression, Anxiety, Pain Intensity, Pain interference, Satisfaction with social role, physical function (e.g., from PROMIS-29), vitality, bodily pain, general health, mental health, physical function (e.g., from SF-36), role emotional, role physical and social function (Table 1). A dataset containing the mean values of the PROs for each patient across the whole study was prepared. After preprocessing, unsupervised clustering analysis of the whole-study PRO means data was carried out. Three metrics of clustering performance were used to assess the best model and optimal number of clusters between Gaussian mixture modeling with variational inference, k-means clustering and hierarchical clustering. This analysis was repeated on a dataset of monthly PRO means where each mean was calculated from PROs recorded in the month preceding a clinic visit.

Unsupervised clustering of ePRO data 122 identified two patient clusters, cluster 1 and cluster 2, from a diverse sample of 62 total SLE patients that differed in their symptoms as assessed by ePRO questionnaires. Cluster 1 contained 51 patients, whereas cluster 2 contained 11 patients. In general, cluster 1 patients reported significantly milder self-reported symptoms (Table 1), compared to cluster 2. Table 1 shows cluster means, for patient clusters obtained using Bayesian Gaussian mixture model for unsupervised clustering of patients by whole-study means of ePRO data. Notably, cluster 1 and 2 patients manifested significantly different SLEDAI as well as Physician Global Assessment (PGA) scores (FIG. 6B). FIG. 6B shows raincloud plots showing differences in the distributions of physician disease activity measures, e.g., SLEDAI and PGA scores, between patients clusters, obtained by clustering 62 patients using whole-study mean ePRO data as inputs. The results were largely repeated in the clustering analysis of monthly mean ePRO data. 408 samples were calculated from the PROs data of 62 patients collected over a multiple months. Clustering of monthly PRO means reveals two patient clusters, a large, healthier cluster (n=338, cluster 1), cluster 1, and a smaller cluster (n=70, cluster 2) with more intense disease activity was obtained (FIGS. 6C and 6D). FIG. 6D shows hierarchical clustering of monthly PRO means reveals two patient clusters, cluster 1 and 2. FIG. 6C shows raincloud plots showing differences in the distributions of physician disease activity measures, e.g., SLEDAI and PGA scores, between patients clusters (cluster 1, and 2 of FIG. 6D), obtained by clustering 408 patients using monthly mean ePRO data as inputs. As shown by the SLEDAI scores, patients in cluster 2 (of FIGS. 6B-C) have active lupus, and patients in cluster 1 (of FIGS. 6B-C) does have active lupus. In FIGS. 6B-C, the asterisks indicate statistical significance calculated from two-sided Mann Whitney-U tests.

*P<0.05; **P<0.01; ***P<0.001; ****P<0.0001. The two groups of SLE patients (cluster 1 and 2) that differ significantly in physician measures of SLE activity, were described by an unsupervised clustering analysis of ePROs collected using a novel smartphone application. Collection of ePRO data and patient cluster assignment based on those data as inputs of a machine learning model could assist health care providers in monitoring SLE patients and personalizing their treatment. FIG. 6E shows ROC and precision recall curves using the ePROs (Table 1) as features, RF classifier was used. No balancing of dataset performed, but only models able to handle unbalanced datasets employed. Classification parameters for FIG. 6E was, AUC: 0.835526, Accuracy: 0.88, Cohen Kappa: 0.671053, Sensitivity: 0.75, Specificity: 0.921,Precision: 0.75, F1 Score: 0.75. FIG. 6F shows ROC and precision recall curves for catboost classifier. Catboost works on categorical representations of the data. Individual items from each PRO form are treated as features (not composite scores) for 26 total features. FIG.6F was generated using the following PRO features, listed by-PRO name: No. of features contributed: Fatigue VAS: 1; Pain VAS: 1; PTGA: 1; Morning stiffness 1; FSS: 9; and FACIT-F: 13.Classification parameters for FIG. 6F was AUC: 0.799, Accuracy: 0.861, Cohen Kappa: 0.646, Sensitivity: 0.64, Specificity: 0.958, Precision: 0.868, F1 Score: 0.737.

FIGS. 6G and H show analysis of the patient clusters obtained by unsupervised clustering of a smaller set of PRO data according to a non-limiting example of the embodiments described herein. Referring to FIG. 6G, the unsupervised clustering may receive as inputs PRO 122 such as social functioning (e.g., from SF-36), Patient Global Assessment, pain intensity (e.g., from PROMIS-29), and mental health (e.g., from SF-36), from patients 110. In a non-limiting example, classification models were trained to distinguish cluster assignment in the monthly means dataset using a smaller subset of the PROs used as features for clustering. Model performance was assessed by area under the receiver operating characteristic curve (AUC) (FIG. 6G). A Support Vector Machine (SVM) classification model was able to classify patients into Group 1 or 2 reliably using only four PROs as input features, including measures of social functioning, Patient Global Assessment, pain intensity, and mental health. The model trained on these four features had a test set AUC of 0.98. Referring to FIG. 6H, the unsupervised clustering may receive as inputs PRO 122 such as FACIT-F, HRQOL, bodily pain (e.g., from SF-36), and Anxiety (e.g., from PROMIS-29), from patients 110. In a non-limiting example, classification models were trained to distinguish cluster assignment in the monthly means dataset using a smaller subset of the PROs used as features for clustering. Model performance was assessed by area under the receiver operating characteristic curve (AUC) (FIG. 6H). The model trained on these four features had a test set AUC of 0.96. The method of obtaining smaller set of PROs may include removing collinear features. Use of a smaller set of PRO questionnaires, may decrease patient frustrations associated with relatively lengthy and/or redundant nature of certain large set of PRO questionnaires, and may increase patient compliance regarding receiving the PRO data from the patient.

In certain embodiments, a primary component analysis, also known as principal component analysis (e.g., the primary component analysis of FIG. 5) may generate cluster plot (e.g., as shown in FIGS. 6A, and 6D) showing one or more clusters based on the PRO and optionally biometric data. In certain embodiments, using the cluster plot, the unsupervised clustering 600A-H may generate a health score plot, such as a SLEDAI plot, for any one or more of the clusters. As shown in FIGS. 6A-H, methods of the current disclosure can classify whether or not a patient has active lupus based on the PRO data from the patient. Based on the clustering of a patient based on the PRO data from the patient, it can be inferred whether the patient has active lupus, or does not have active lupus. Such clustering can be used for monitoring and personalizing their treatment. For as example, based on cluster assignment of the patient an action item, method of evaluation and method of treatment may be generated, recommended, and/or selected for the patient.

TABLE 1
Numerical summary of patient clusters.

| | | Cluster means | | |
|---|---|---|---|---|
| | | Cluster 1 | Cluster 2 | |
| Survey | Input variables | (n = 51) | (n = 11) | P-value |
| SLAQ | SLAQ | 9.04 | 21.0 | <0.001 |
| LupusPRO | HRQOL | 73.26 | 44.4 | <0.001 |
| | Non-HRQOL | 71.81 | 57.97 | <0.01 |
| VAS | Fatigue VAS | 36.32 | 63.68 | <0.001 |
| | Pain VAS | 28.35 | 59.81 | <0.001 |
| | PtGA | 33.41 | 62.40 | <0.001 |
| FSS | FSS | 4.00 | 5.73 | <0.01 |
| FACIT-F | FACIT-F | 33.72 | 22.49 | <0.001 |
| Morning Stiffness | Morning Stiffness | 1.45 | 2.73 | <0.01 |
| PROMIS-29 | Fatigue | 56.77 | 66.19 | <0.01 |
| | Sleep Disturbance | 53.29 | 61.86 | <0.01 |
| | Depression | 48.71 | 63.71 | <0.001 |
| | Anxiety | 49.07 | 64.72 | <0.001 |
| | Pain Intensity | 31.13 | 62.73 | <0.001 |
| | Pain Interference | 55.37 | 66.79 | <0.001 |
| | Satisfaction w/ | 44.08 | 33.86 | <0.001 |
| | Social Role | | | |
| | Physical Function | 44.00 | 34.34 | <0.001 |
| SF-36 | Vitality | 39.79 | 21.91 | <0.01 |
| | Bodily Pain | 63.99 | 32.39 | <0.001 |
| | General Health | 35.01 | 14.38 | <0.001 |
| | Mental Health | 77.53 | 43.59 | <0.001 |
| | Physical Function | 59.71 | 26.80 | <0.001 |
| | Role Emotional | 76.35 | 8.62 | <0.001 |
| | Role Physical | 27.22 | 0.16 | <0.001 |
| | Social Function | 68.16 | 19.47 | <0.001 |

Note.
Bayesian Gaussian mixture model used for unsupervised clustering of patients by whole-study means of ePRO data. P-values obtained from two-sided Mann-Whitney U tests.

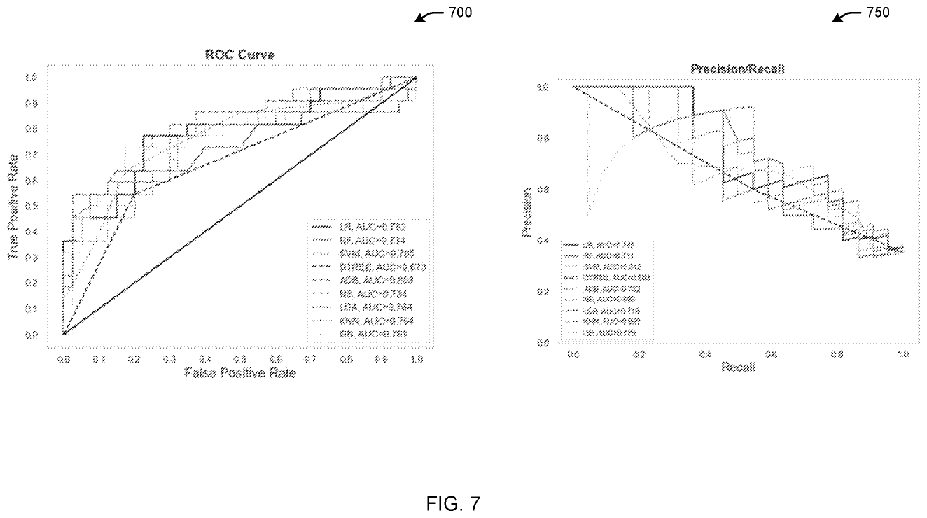

FIG. 7 shows non-limiting examples of curves for analyzing PRO data according to embodiments described herein.

Referring to FIG. 7, a receiver operating characteristic (ROC) curve 700 and a precision/recall curve 750 are shown, and display the capability of PRO surveys to classify whether a patient has active (e.g., SLEDAI≥6) or inactive (e.g., SLEDAI<6) disease activity as measured by a physician. Models may be trained and tested using paper-collected PRO scores as features: Patient Global Assessment (PtGA), pain and fatigue as measured using a visual analogy scale (VAS), morning stiffness, FSS, FACIT-F, SF-36 (8 domain scores), PROMIS-29 (8 domain scores), LupusPRO (2 construct scores) and SLAQ. Nine machine learning classifiers were performed using 10-fold cross validation including Logistic Regression (LR), Random Forest (RF), Support Vector (SVM), Decision Trees (DTREE), Ada Boost (ADB), Naïve Bayes (NB), Linear Discriminant Analysis (LDA), K-Nearest Neighbors (KNN) and Gradient Tree Boosting (GB). RF and GB classifiers both resulted in a sensitivity>0.64, specificity>0.82, and kappa>0.50.

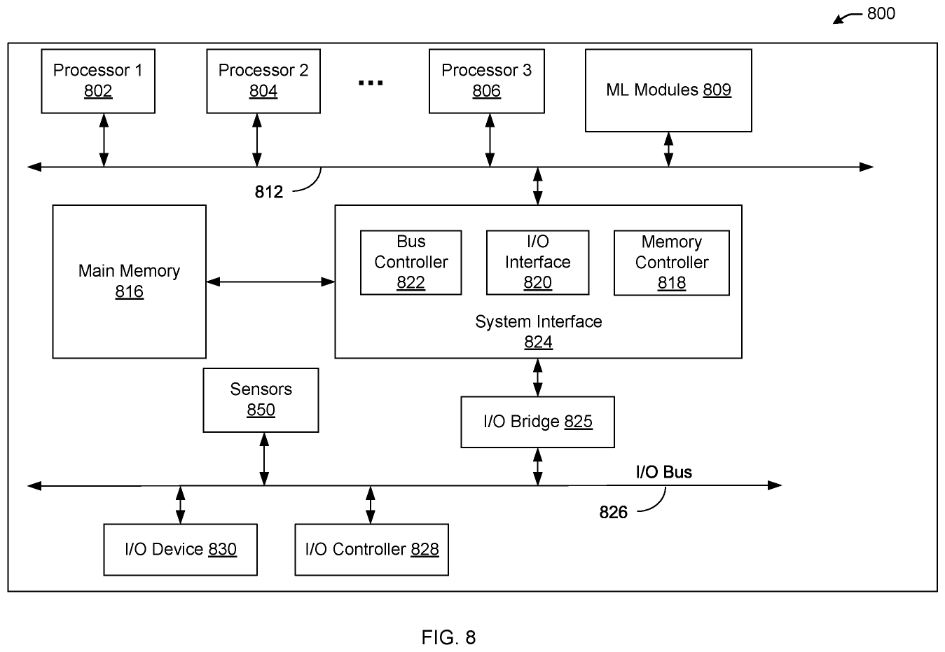

FIG. 8 is a block diagram illustrating an example of a computing device or computer system 800 upon which any of one or more techniques (e.g., methods) may be performed, in accordance with one or more example embodiments of the present disclosure.

For example, the computing system 800 of FIG. 5 may represent the devices 102 and/or the one or more remote devices 120 of FIG. 1. The computer system 800 (system) includes one or more processors 802-806 and one or more machine learning (ML) modules 809 (e.g., capable of performing the processes of FIGS. 4-6 and generating the curves of FIG. 7). Processors 802-806 may include one or more internal levels of cache (not shown) and a bus controller (e.g., bus controller 822) or bus interface (e.g., I/O interface 820) unit to direct interaction with the processor bus 812. According to one embodiment, the processors 802-806 may include tensor processing units (TPUs) and/or other artificial intelligence accelerator application-specific integrated circuits (ASICs) that may allow for neural networking and other machine learning used to perform the enhanced operations described herein (e.g., FIGS. 4-7). The computing system 800 may include one or more applications (e.g., when implemented as the devices 104 and/or 106 of FIG. 1).

Processor bus 512, also known as the host bus or the front side bus, may be used to couple the processors 802-806 with the system interface 824. System interface 824 may be connected to the processor bus 812 to interface other components of the system 500 with the processor bus 812. For example, system interface 824 may include a memory controller 818 for interfacing a main memory 816 with the processor bus 812. The main memory 816 typically includes one or more memory cards and a control circuit (not shown). System interface 824 may also include an input/output (I/O) interface 820 to interface one or more I/O bridges 825 or I/O devices 830 with the processor bus 812. One or more I/O controllers and/or I/O devices may be connected with the I/O bus 826, such as I/O controller 828 and I/O device 830, as illustrated.

I/O device 830 may also include an input device (not shown), such as an alphanumeric input device, including alphanumeric and other keys for communicating information and/or command selections to the processors 802-806. Another type of user input device includes cursor control, such as a mouse, a trackball, or cursor direction keys for communicating direction information and command selections to the processors 802-806 and for controlling cursor movement on the display device.

System 800 may include a dynamic storage device, referred to as main memory 816, or a random access memory (RAM) or other computer-readable devices coupled to the processor bus 812 for storing information and instructions to be executed by the processors 802-806. Main memory 816 also may be used for storing temporary variables or other intermediate information during execution of instructions by the processors 802-806. System 800 may include read-only memory (ROM) and/or other static storage device coupled to the processor bus 512 for storing static information and instructions for the processors 802-806. The system outlined in FIG. 8 is but one possible example of a computer system that may employ or be configured in accordance with aspects of the present disclosure.

According to one embodiment, the above techniques may be performed by computer system 800 in response to processor 804 executing one or more sequences of one or more instructions contained in main memory 816. These instructions may be read into main memory 816 from another machine-readable medium, such as a storage device. In alternative embodiments, circuitry may be used in place of or in combination with the software instructions. Thus, embodiments of the present disclosure may include both hardware and software components.

The computer system 800 may include sensors 850, which may include biometric sensors (e.g., heart rate sensors, breathing sensors, body temperature sensors, and the like) and/or device motion sensors (e.g., accelerometers, magnetometers, etc.).

Various embodiments may be implemented fully or partially in software and/or firmware. This software and/or firmware may take the form of instructions contained in or on a non-transitory computer-readable storage medium. Those instructions may then be read and executed by one or more processors to enable the performance of the operations described herein. The instructions may be in any suitable form, such as, but not limited to, source code, compiled code, interpreted code, executable code, static code, dynamic code, and the like. Such a computer-readable medium may include any tangible non-transitory medium for storing information in a form readable by one or more computers, such as but not limited to read-only memory (ROM); random access memory (RAM); magnetic disk storage media; optical storage media; a flash memory, etc.

A machine-readable medium includes any mechanism for storing or transmitting information in a form (e.g., software, processing application) readable by a machine (e.g., a computer). Such media may take the form of, but is not limited to, non-volatile media and volatile media and may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, solid state devices (SSDs), and the like. The one or more memory devices (not shown) may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in main memory 516, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

The operations and processes described and shown above may be carried out or performed in any suitable order as desired in various implementations. Additionally, in certain implementations, at least a portion of the operations may be carried out in parallel. Furthermore, in certain implementations, less than or more than the operations described may be performed.

The word “exemplary” is used herein to mean “serving as an example, instance, or illustration.” Any embodiment described herein as “exemplary” is not necessarily to be construed as preferred or advantageous over other embodiments.

As used herein, unless otherwise specified, the use of the ordinal adjectives “first,” “second,” “third,” etc., to describe a common object, merely indicates that different instances of like objects are being referred to and are not intended to imply that the objects so described must be in a given sequence, either temporally, spatially, in ranking, or any other manner.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Reference in the specification to “embodiments,” “certain embodiments,” “preferred embodiments,” “specific embodiments,” “some embodiments,” “an embodiment,” “one embodiment” or “other embodiments” mean that a particular feature, structure, or characteristic described in connection with the embodiments is included in at least some embodiments, but not necessarily all embodiments, of the present disclosure.

It is understood that the above descriptions are for purposes of illustration and are not meant to be limiting.

The disclosure includes the following non-limiting embodiments.

Embodiment 1 may include a method for determining a method of evaluation and treatment for patients, the method including: receiving, by at least one processor of a first device, patient-reported outcome data from a second device, the patient-reported outcome data indicative of at least one of a patient's reactions to a disease of the patient or a first method of evaluation or treatment using a pharmaceutical or biological treatment; validating, by the at least one processor, the patient-reported outcome data; inputting, by the at least one processor, the patient-reported outcome data to a first machine learning model; generating, using the first machine learning model, based on the patient-reported outcome data, a score indicative of at least one of an activity of the patient's disease, an effectiveness of current treatment, or severity a of the patient's reactions; and, optionally, generating, using a second machine learning model, based on the score, a second method of evaluation or treatment for the patient.

Embodiment 2 may include the method of embodiment 1 and/or some other embodiment herein, further including training the first machine learning model to generate the score based on a percentage of scores associated with adjusting the first method of evaluation or treatment.

Embodiment 3A may include the method of embodiment 1 and/or some other example herein, wherein generating the second method of evaluation or treatment for the patient is further based on a comparison of the score to a score threshold.

Embodiment 3B may include the method of embodiment 1 and/or some other embodiment herein, wherein the second method of evaluation or treatment is different than the first method of evaluation or treatment.

Embodiment 4 may include the method of embodiment 1 and/or some other embodiment herein, wherein the second method of evaluation or treatment is different than the first method of evaluation or treatment.

Embodiment 5 may include the method of embodiment 1 and/or some other embodiment herein, wherein the second method of evaluation or treatment is the same as the first method of evaluation or treatment.

Embodiment 6 may include the method of embodiment 1 and/or some other embodiment herein, further including receiving at least one of biometric data or device motion data, wherein generating the score is further based on the at least one of the biometric data or the device motion data.

Embodiment 7 may include the method of embodiment 6 and/or some other embodiment herein, wherein the biometric data comprises at least one of sleep data, breathing data, body temperature data, or heart rate data.

Embodiment 8 may include the method of embodiment 6 and/or some other embodiment herein, wherein the device motion data comprises accelerometer data indicative of activity of the patient.

Embodiment 9 may include a method for predicting a Physician's Global Assessment Score, the method including: receiving, by at least one processor of a first device, patient-reported outcome data from a second device, the patient reported outcome data indicative of a patient's disease status or reactions of the patient to a first method of treatment using a pharmaceutical or biological treatment; validating, by the at least one processor, the patient-reported outcome data; receiving, by the at least one processor, documents associated with visits to a doctor; inputting, by the at least one processor, the patient-reported outcome data and the documents to a first machine learning model; generating, using the first machine learning model, based on the patient-reported outcome data and the documents, a prediction of a Physician's Global Assessment Score indicative of a patient's status or a severity of the patient's reactions; and optionally generating, using a second machine learning model and the prediction of the Physician's Global Assessment Score, a second method of treatment for the patient.

Embodiment 10 may include the method of embodiment 9 and/or some other embodiment herein, further including training the first machine learning model to generate the score based on a percentage of scores associated with adjusting the first method of evaluation or treatment.

Embodiment 11 may include the method of embodiment 9 and/or some other embodiment herein, wherein generating the second method of evaluation or treatment for the patient is further based on a comparison of the score to a score threshold.

Embodiment 12 may include the method of embodiment 9 and/or some other embodiment herein, wherein the second method of evaluation or treatment is different than the first method of evaluation or treatment.

Embodiment 13 may include the method of embodiment 9 and/or some other embodiment herein, wherein the second method of evaluation or treatment is the same as the first method of evaluation or treatment.

Embodiment 14 may include the method of embodiment 9 and/or some other embodiment herein, further including receiving at least one of biometric data or device motion data, wherein generating the score is further based on the at least one of the biometric data or the device motion data.

Embodiment 15 may include a method of treatment for patients having Lupus, the method including: receiving, by at least one processor of a first device, patient-reported outcome data from a second device, the patient-reported outcome data indicative of a patient's reactions to a method of evaluation or treatment for Lupus using a pharmaceutical or biological treatment; validating, by the at least one processor, the patient-reported outcome data; receiving, by the at least one processor, documents associated with visits to a doctor; inputting, by the at least one processor, the patient-reported outcome data and the documents to a first machine learning model; generating, using the first machine learning model, based on the patient-reported outcome data and the documents, a prediction of a Systematic Lupus Erythematosus Disease Activity Index (SLEDAI) Score indicative of activity of a patient's disease and the patient's reactions; and optionally adjusting, using a second machine learning model, based on the prediction of the SLEDAI Score, the method of evaluation or treatment for the patient.

Embodiment 16 may include the method of embodiment 15 and/or some other embodiment herein, further including training the first machine learning model to generate the score based on a percentage of scores associated with adjusting the first method of evaluation or treatment.

Embodiment 17 may include the method of embodiment 15 and/or some other embodiment herein, wherein generating the second method of evaluation or treatment for the patient is further based on a comparison of the score to a score threshold.

Embodiment 18 may include the method of embodiment 15 and/or some other embodiment herein, wherein the second method of evaluation or treatment is different than the first method of evaluation or treatment.

Embodiment 19 may include the method of embodiment 15 and/or some other embodiment herein, wherein the second method of evaluation or treatment is the same as the first method of evaluation or treatment.

Embodiment 20 may include the method of embodiment 15 and/or some other embodiment herein, further including receiving at least one of biometric data or device motion data, wherein generating the SLEDAI score is further based on the at least one of the biometric data or the device motion data.

Embodiment 21 may include a method for evaluation and/or treatment of a disease in a patient, the method comprising: receiving, by at least one processor of a first device, patient-reported outcome data from a second device, the patient-reported outcome data indicative of at least one of a patient's reactions to patient's health, or a first method of evaluation or treatment using a pharmaceutical or biological treatment; validating, by the at least one processor, the patient-reported outcome data; inputting, by the at least one processor, the patient-reported outcome data to a first machine learning model; generating, using the first machine learning model, based on the patient-reported outcome data, i) a score indicative of at least one of an activity of the patient's disease, an effectiveness of current treatment, or severity of the patient's reactions, and/or ii) an inference indicative of the disease state of the patient; and generating, recommending, and/or selecting based on the score and/or the inference, an action item and/or a second method of evaluation or treatment for the patient, optionally using a second machine learning model.

Embodiment 22 may include the embodiment of 21, further comprising: training the first machine learning model to generate the score based on a percentage of scores associated with adjusting the first method of evaluation or treatment.

Embodiment 23 may include embodiment 21 or 22, wherein generating, recommending, and/or selecting the action item and/or the second method of evaluation or treatment for the patient is further based on a comparison of the score to a score threshold.

Embodiment 24 may include any one of embodiments 21 to 23, wherein the second method of evaluation or treatment is different than the first method of evaluation or treatment.

Embodiment 25 may include any one of embodiments 21 to 23, wherein the second method of evaluation or treatment is the same as the first method of evaluation or treatment.

Embodiment 26 may include any one of embodiments 21 to 25, further comprising: receiving at least one of biometric data or device motion data, wherein generating the score and/or inference is further based on the at least one of the biometric data or the device motion data.

Embodiment 27 may include embodiment 26, wherein the biometric data comprises at least one of sleep data, breathing data, body temperature data, or heart rate data.

Embodiment 28 may include embodiment 26 or 27, wherein the device motion data comprises accelerometer data indicative of activity of the patient.

Embodiment 29 may include any one of embodiments 21 to 28, wherein the inference is, whether the PRO data is indicative of the patient having active disease, or not having active disease.

Embodiment 30 may include any one of embodiments 21 to 29, wherein the action item comprises scheduling for the patient an appointment, visit, and/or consultation with a healthcare professional.

Embodiment 31 may include any one of embodiments 21 to 30, further comprising performing the action item, performing the second method of evaluation, and/or administering the second treatment to the patient.

Embodiment 32 may include a method for predicting a Physician's Global Assessment Score, the method comprising: receiving, by at least one processor of a first device, patient-reported outcome data from a second device, the patient reported outcome data indicative of a patient's disease status or reactions of the patient to a first method of evaluation or treatment using a pharmaceutical or biological treatment; validating, by the at least one processor, the patient-reported outcome data; receiving, by the at least one processor, documents associated with visits to a doctor; inputting, by the at least one processor, the patient-reported outcome data and the documents to a first machine learning model; generating, using the first machine learning model, based on the patient-reported outcome data and the documents, a prediction of a Physician's Global Assessment Score indicative of a patient's status or a severity of the patient's reactions; and generating, recommending, and/or selecting based on the prediction of the Physician's Global Assessment Score, an action item, and/or a second method of evaluation or treatment for the patient, optionally using a second machine learning model.

Embodiment 33 may include the embodiment 32, further comprising: training the first machine learning model to generate the Physician's Global Assessment Score based on a percentage of scores associated with adjusting the first method of evaluation or treatment.

Embodiment 34 may include any one of embodiments 32 to 33, wherein generating the second method of evaluation or treatment for the patient is further based on a comparison of the Physician's Global Assessment Score to a score threshold.

Embodiment 35 may include any one of embodiments 32 to 34, wherein the second method of evaluation or treatment is different than the first method of evaluation or treatment.

Embodiment 36 may include any one of embodiments 21 to 34, wherein the second method of evaluation or treatment is the same as the first method of evaluation or treatment.

Embodiment 37 may include any one of embodiments 21 to 36, further comprising: receiving at least one of biometric data or device motion data, wherein generating the Physician's Global Assessment Score is further based on the at least one of the biometric data or the device motion data.

Embodiment 38 may include, a method for diagnosis and/or treating lupus in a patient, the method comprising: receiving, by at least one processor of a first device, patient-reported outcome data from a second device, the patient-reported outcome data indicative of a patient's reactions to i) patient's health, ii) to a first method of evaluation and/or ii) to a first method of treatment for Lupus using a pharmaceutical or biological treatment; validating, by the at least one processor, the patient-reported outcome data; optionally receiving, by the at least one processor, a doctor's data comprising data from the patient's visits to a doctor; inputting, by the at least one processor, the patient-reported outcome data and optionally the doctor's data to a first machine learning model; generating, using the first machine learning model, based on the patient-reported outcome data and optionally the doctor's data, i) a score indicative of activity of a patient's disease and the patient's reactions, and/or ii) an inference indicative of the lupus disease state of the patient; and adjusting, generating, recommending, and/or selecting, based on the score and/or the inference, an action item and/or a second method of evaluation or treatment for the patient, optionally using a second machine learning model.

Embodiment 39 may include embodiments 38, further comprising: training the first machine learning model to generate the SLEDAI score based on a percentage of scores associated with adjusting the first method of evaluation or treatment.

Embodiment 40 may include any one of embodiments 38 to 39, wherein generating the second method of evaluation or treatment for the patient is further based on a comparison of the score SLEDAI to a score threshold.

Embodiment 41 may include any one of embodiments 38 to 40, wherein the second method of evaluation or treatment is different than the first method of evaluation or treatment.

Embodiment 42 may include any one of embodiments 38 to 40, wherein the second method of evaluation or treatment is the same as the first method of evaluation or treatment.

Embodiment 43 may include any one of embodiments 38 to 42, further

comprising: receiving at least one of biometric data or device motion data, wherein generating the SLEDAI score and/or the inference is further based on the at least one of the biometric data or the device motion data.

Embodiment 44 may include any one of embodiments 38 to 43, wherein the inference is whether the PRO data from the patient is indicative of the patient having active lupus, or not having active lupus.

Embodiment 45 may include any one of embodiments 38 to 44, wherein the PRO data comprises one or more of SLAQ data, HRQOL data, Non-HRQOL data, Fatigue VAS data, Pain VAS data, PtGA data, FSS data, FACIT-F data, Morning Stiffness data, Fatigue data, Sleep disturbance data, Depression data, Anxiety data, Pain Intensity data, Pain interference data, Satisfaction with social role data, physical function data, vitality data, bodily pain data, general health data, mental health data, physical function data, role emotional data, role physical data and social function data.

Embodiment 46 may include any one of embodiments 38 to 45, wherein the PRO data comprises PtGA data, Pain Intensity data, mental health data, and social function data.

Embodiment 47 may include any one of embodiments 38 to 46, wherein the first machine learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Embodiment 48 may include any one of embodiments 38 to 47, wherein the first machine learning model generate the score and/or the inferenceusing linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

Embodiment 49 may include any one of embodiments 38 to 48, wherein the action item comprises scheduling for the patient an appointment, visit, and/or consultation with a healthcare professional.

Embodiment 50 may include any one of embodiments 38 to 49, further comprising performing the action item, performing the second method of evaluation, and/or administering the second treatment to the patient.

Embodiment 51 may include any one of embodiments 38 to 50, wherein the second treatment is configured to treat active lupus.

Embodiment 52 may include any one of embodiments 38 to 51, wherein the second treatment is configured to treat reduce severity of active lupus.

Embodiment 53 may include any one of embodiments 38 to 52, wherein the second treatment is configured to reduce risk of having active lupus

Embodiment 54 may include any one of embodiments 38 to 53, wherein the patient has lupus.

Embodiment 55 may include an apparatus comprising means for: receiving patient-reported outcome data from a second device, the patient-reported outcome data indicative of at least one of a patient's reactions to i) the patient's health, ii) a disease of the patient and/or iii) a first method of evaluation or treatment using a pharmaceutical or biological treatment; validating the patient-reported outcome data; inputting the patient-reported outcome data to a first machine learning model; generating, using the first machine learning model, based on the patient-reported outcome data, i) a score indicative of at least one of an activity of the patient's disease, an effectiveness of current treatment, or severity a of the patient's reactions, and/or ii) an inference indicative of the disease state of the patient; and, optionally, generating, recommending, and/or selecting, based on the score and/or the inference, an action item and/or a second method of evaluation or treatment for the patient, optionally using a second machine learning model.

Embodiment 56 may include one or more non-transitory computer-readable media comprising instructions to cause an electronic device, upon execution of the instructions by one or more processors of the electronic device, to perform one or more elements of a method described in or related to any of embodiments 1-55, or any other method or process described herein

Embodiment 57 may include an apparatus comprising logic, modules, and/or circuitry to perform one or more elements of a method described in or related to any of embodiments 1-55, or any other method or process described herein.

Embodiment 58 may include a method, technique, or process as described in or related to any of embodiments 1-55, or portions or parts thereof.

Embodiment 59 may include an apparatus comprising: one or more processors and one or more computer readable media comprising instructions that, when executed by the one or more processors, cause the one or more processors to perform the method, techniques, or process as described in or related to any of embodiments 1-55, or portions thereof.

Embodiments according to the disclosure are in particular disclosed in the attached claims directed to a method, a storage medium, a device and a computer program product, wherein any feature mentioned in one claim category, e.g., method, can be claimed in another claim category, e.g., system, as well. The dependencies or references back in the attached claims are chosen for formal reasons only. However, any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Unless otherwise defined, all technical terms used herein have the same meaning as commonly understood by one of ordinary skill in the art to which this invention belongs.

As used herein, the singular forms “a,” “an,” and “the” include plural references unless the context clearly dictates otherwise. Any reference to “or” herein is intended to encompass “and/or” unless otherwise stated.

As used herein, the term “about,” and “substantially” refers to an amount that is near the stated amount by about ±10%, ±5%, or ±1%, including increments therein.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

### EXAMPLES

The following illustrative examples are representative of embodiments of the software applications, systems, and methods described herein and are not meant to be limiting in any way.

### Example 1: Patient-Reported Outcome Information Collected from Lupus Patients Using a Mobile Application: Compliance and Validation

Systemic lupus erythematosus (SLE) is a chronic autoimmune disease characterized by diverse manifestations and clinical heterogeneity (1). Patients with active SLE experience a range of clinical manifestations, and lupus is often complicated by flares of varying severity, followed by periods of clinical quiescence (2). Even during times of lesser inflammatory activity, lupus patients frequently experience varying levels of symptoms such as daily fluctuations in fatigue or pain (1). Consequently, individuals living with SLE face a lifetime of symptomatic burdens, including fatigue, pain, sleep disturbance, and neuropsychiatric manifestations, that impair their ability to carry out normal daily activities and contribute to a reduction in health-related quality of life (HRQOL) (3). The detrimental impacts of SLE on HRQOL are often undervalued in physician assessments of disease activity and damage, causing frequent discordance between physicians' and SLE patients' estimations of disease burden (4). As a result, patient-reported outcomes (PROs) in general consist of structured feedback directly from patients regarding their symptoms, which can be used to supplement other more standard clinical measures such as the physician-reported SLE Disease Activity Index (SLEDAI) (5). PRO instruments capture critical information uniquely known to the patient, such as fatigue, pain, memory loss, emotional well-being, and anxiety level, and have been shown to provide insight regarding treatment effectiveness and mortality prediction in SLE patients (6). Both disease agnostic HRQOL tools (such as the Medical Outcomes Short Form 36 [SF-36]) and SLE-specific PRO instruments (such as the LupusPRO questionnaire) (7) have been developed to evaluate the impact of disease on an individual patient.

One issue with current instruments is that PROs are often recorded intermittently, typically during an in-person clinic visit, and require patients to recall a period of several weeks or months. Consequently, important PRO information may not always be accurate or representative of the complete recall period. Administration of paper-and-pencil questionnaires require data to be collected, recorded, and computerized manually, which limits the ability to perform timely analysis and may lead to secondary data entry errors (8, 9).

Clinically focused mobile health applications (apps) have been developed previously that use ePRO surveys to monitor other inflammatory diseases such as rheumatoid arthritis and report high (79%) median patient adherence (10, 11). We have developed a custom-designed smartphone app for the purpose of dense PRO monitoring to facilitate the analysis of real-time trends in SLE patient self-assessment. The current report includes a relatively long-duration follow-up (6 months) of SLE patients to assess the utility of our app in the remote symptom reporting of various PRO instruments. This study sought to evaluate patient compliance with mobile app PRO completion and determine the variability and/or equivalency of measurements derived from digital measures compared with traditional paper PROs. Taken together, these analyses demonstrate that PRO data collected via the mobile app are reliable and suggest that this information can be used within the context of a clinical trial or in clinical practice as a means to catalog real-time changes in disease status and support timely therapeutic interventions.

### Materials and Methods

PRO measurement tools: Participants were instructed to use a 100-mm visual analog scale (VAS) daily with the app and at every site visit to measure patient global assessment (PtGA), fatigue (Fatigue), and pain (Pain). VASs allow continuous scaling of disease severity, directly grounded in clinical observation at the time of scoring (12).

Subjects were prompted to report the duration of morning stiffness each day by noon via smartphone and at clinic visits on paper. Although this is not a standard PRO measure, the morning stiffness ePRO was created for this trial because morning symptoms are typical of an inflammatory disease and are improved by RAYOS (Horizon Pharma) in rheumatoid arthritis (13). On weekdays and during clinic visits, patients completed the following fatigue assessments: the Fatigue Severity Scale (FSS; 9 questions) (14) and the Functional Assessment of Chronic Illness Therapy-Fatigue Scale (FACIT-F Version 4; 13 questions) (15). The SF-36 (36 questions; 8 scored domains) (16), the Patient Reported Outcome Measurement Information System (PROMIS-29 Profile Version 1.0; 29 questions; 7 scored domains) (17), and the LupusPRO (Version 1.8) survey (49 questions; 12 scored domains; 2 constructs) (7) were also completed once a week and on paper during in-clinic visits to evaluate effect of disease burden on quality of life. The Systemic Lupus Activity Questionnaire (SLAQ) (5), which is not a PRO but rather a personal assessment of SLE disease activity, was also completed weekly via the app and at clinic visits on paper. For FACIT-F, SF-36, and LupusPRO, higher scores indicate better health; for PtGA, Fatigue, Pain, Morning Stiffness, FSS, and SLAQ, higher scores indicate a negative impact on health.

eLuPRO development: The eLuPRO mobile device app was designed with input from both physician and patient focus groups. The mobile PRO app (hereafter referred to as “eLuPRO”) was prepared in JavaScript for use on the android platform. The eLuPRO app was mounted on a Galaxy S7 smartphone (Samsung Electronics), which was provided to each subject for the duration of the study with entries uploaded daily to a secure database. The app data were stored in an independent database contained on a Health Insurance Portability and Accountability Act (HIPAA)-compliant secure cloud-based server. The app was created with content from the validated PRO instruments described subsequently. The individual PRO instruments were reproduced in English identically for the app except that the wording of several PRO instruments (FSS, FACIT-F, SF-36, LupusPRO Version 1.8, and SLAQ) was modified during eLuPRO development to account for the revised recall periods used for this study (ie, “the past 24 hours or the past week”). Questions using the VAS were oriented in landscape when displayed on the phone such that the scale was 100 mm in length as per the standard paper version. For every survey, one question was asked per screen, and a green check mark appeared on the eLuPRO home screen once a PRO was completed. Patients were instructed to bring the smartphone to all activities, including walks, errands, and trips. Daily reminders were set on the smartphone and via a paired Samsung Gear S2 Smartwatch to prompt PRO data entry. The reminder for the morning stiffness questionnaire was sent daily at noon; however, all other PROs were completed in the evening to capture the full day's variations. Patients were able to customize the exact time at which they were reminded to complete evening surveys.

Study design: The eLuPRO app was evaluated as part of an exploratory study conducted within the completed phase 4 RIFLE trial (RAYOS Inhibits Fatigue in Lupus Erthymatosus; www.ClinicalTrials.gov identifier NCT03098823), which was a multicenter, randomized, double-blind, double dummy crossover study comparing the effect of delayed release prednisone (RAYOS) on fatigue in SLE with the effect of immediate-release (IR) prednisone on fatigue in SLE. This study recruited 62 SLE patients aged 18 years or older between Sep. 12, 2017, and May 28, 2019. Participants were required to meet SLE classification criteria defined by either the American College of Rheumatology (ACR) or the Systemic Lupus International Collaborating Clinics Classification (SLICC), to have increased fatigue as assessed by a FACIT-F score of less than 25, and to be on a stable regimen of IR prednisone before screening. All patients were either English-speaking or had a caregiver who spoke English. During the 26-week trial, participants were instructed to use the custom-built mobile app eLuPRO to complete PRO surveys daily, weekly, or 5 days a week according to a provided PRO schedule. eLuPRO tracking additionally included a 14-day lead-in period to establish baseline disease activity and confirm eHealth literacy. Patients unable to use the eLuPRO app during baseline were removed. In-clinic visits occurred at two baseline visits and monthly for the duration of the study, during which patients completed both paper and eLuPRO versions of all PRO instruments separated by a distraction (participants were given lunch). Paper responses were manually entered into an electronic database, and data were securely stored in the study electronic data collection system (iMedNet), whereas PRO responses were entered into a database via the smartphone daily. The study was approved by the Institutional Review Board (IRB) at each clinical site, and patients agreed to participate by signing an IRB-approved informed consent form.

Health literacy was assessed using a validated 3-item measure developed by Chew et al (18). For our analyses, participants who responded “sometimes,” “usually,” or “always” for questions 1 or 2, or “somewhat,” “a little bit,” or “not at all” to question 3, were classified as having limited health literacy, as described in Katz et al (19).

### Statistical Analysis

Compliance: Compliance (completing PROs according to the survey schedule) for all surveys was computed by expressing the number of PROs completed on the specified days as a percentage of how many should have been completed given the subjects' enrollment and completion/withdrawal date. A Friedman's analysis of variance (ANOVA) test was employed to evaluate significant differences in the mean rank of compliance across surveys. Application of the Wilcoxon signed rank test additionally evaluated pairwise significance using the Bonferroni P value adjustment. Compliance of patients who completed the trial was also assessed weekly to determine whether app use fluctuated over time.

Cronbach alpha: For multiquestion PRO tools, Cronbach alpha coefficients were computed as a measure of intersurvey reliability. Alpha coefficients for each PRO tool were calculated separately for the paper and electronic modes using all available data between baseline and trial completion. Results for paper and ePROs were tabulated and compared with previously published Cronbach coefficients for each PRO instrument in order to assess similarity. No direct statistical comparisons of coefficients were made between measurement methods; however, we examined these values to assess whether using eLuPRO distorted the internal consistency of the PROs.

eLuPRO and paper PRO comparability: To assess the equivalence between administration methods, we used data from the 8 clinic visits for which same-day electronic and paper PRO responses were recorded. Domains for each measurement tool were assessed independently, and all comparisons were performed on a by-visit basis as well as were summarized on an overall, combined-visit basis. The strength of association was tested using multiple statistical approaches, including pair-wise Student's t tests, intraclass correlation coefficients (ICCs), Pearson's correlation coefficients, and Bland-Altman plots displaying agreement and bias. Pairwise Student's t tests were performed to examine whether there was a statistically significant difference between the mean score of the two administration methods. Level of agreement was evaluated statistically by ICCs, with the absolute agreement denoted at each of the 8 study visits (20). Box-plots were produced to compare the distribution of PRO scores. Scatterplots with a fitted least squares regression line were created, and Pearson's correlation coefficients were calculated to evaluate the linear relationship between paper-based and mobile-app-based PRO scores. Agreement and bias between collection methods were shown graphically by Bland-Altman plots, which plot the score difference (electronic minus paper) against the mean paper and electronic score for each individual (21). The Bland-Altman plots include horizontal reference lines for the mean of the difference in the modes, the mean plus and minus twice the standard deviation for 95% limits of agreement, and a zero-reference line (21).

### Results

Data collection: PRO data were collected from the 62 SLE patients enrolled in the study from 21 sites across the United States (www.ClinicalTrials.gov identifier NCT03098823). A total of 46 subjects completed the entire 6-month study. Of the 16 subjects withdrawn, 11 did so within the first 3 months of the trial. Over the duration of the study, 58,173 PROs were collected through the eLuPRO app, along with 4,374 paper surveys from all clinic visits. This included 263 instances in which paper and eLuPRO versions were completed at the clinic site separated by a distraction (usually lunch), according to protocol.

Patient demographics: Enrolled subjects included 57 females (91.9%) and 5 males (8.1%) from diverse self-reported ancestral backgrounds, as detailed in Table 2. The subject population had a mean age of 45.7 years and had attained an average of 15.6 years of education where 16 years represents an individual who completed a 4-year college degree. Based on the responses to three validated health literacy questions (18), patients were adequately health literate, with 93.5% (58/62) of patients “rarely” or “never” experiencing problems learning about their condition because of difficulty understanding the information, 91.9% (57/62) of patients “rarely” or “never” receiving assistance reading health plan materials, and 97% (60/62) of patients being “extremely” or “quite a bit” confident in filling out medical forms without further assistance.

TABLE 2
Demographic details of enrolled study participants

| Sex | Female | 57/41 | N/A |
|---|---|---|---|
| | (enrolled/completed) | | |
| | Male | 5/5 | |
| | (enrolled/completed) | | |
| Age at baseline, y | 20-29 | 7 | 45.7 |
| | 30-39 | 13 | |
| | 40-49 | 20 | |
| | 50-59 | 13 | |
| | 60-69 | 8 | |
| | 70-89 | 1 | |
| Ethnicity | Hispanic | 11 | N/A |
| | Non-Hispanic | 51 | |
| Race | American | 0 | N/A |
| | Indian/Alaska native | | |
| | Native | 0 | |
| | Hawaiian/Pacific | | |
| | Islander | | |
| | Asian | 3 | |
| | Black or African | 18 | |
| | ancestry | | |
| | White | 37 | |
| | Mixed race or other | 4 | |
| Education in years | 12 (H.S.) | 8 | 15.6 |
| | 13-16 (college) | 35 | |
| | 17-20 (post-graduate) | 21 | |
| Health literacy | Response | | |
| Question 1: “How often do you have | Always | 0 | N/A |
| problems learning about your medical | Usually | 0 | |
| condition because of difficulty | Sometimes | 4 | |
| understanding written information?” | Rarely* | 8 | |
| | Never* | 50 | |
| Question 2: “How often do you have someone | Always | 0 | N/A |
| like a family member, friend, hospital or clinic | Usually | 1 | |
| worker, or caregiver help you read health | Sometimes | 4 | |
| plan materials, such as written information | Rarely* | 7 | |
| about your health or care you are offered?” | Never* | 50 | |
| Question 3: “How confident are you filling | Not at all | 0 | |
| out medical forms by yourself?” | A little bit | 1 | |
| | Somewhat | 1 | |
| | Quite a bit* | 6 | |
| | Extremely* | 54 | |

Abbreviations: H.S., high school; N/A, not applicable. *Response indicates adequate health literacy.

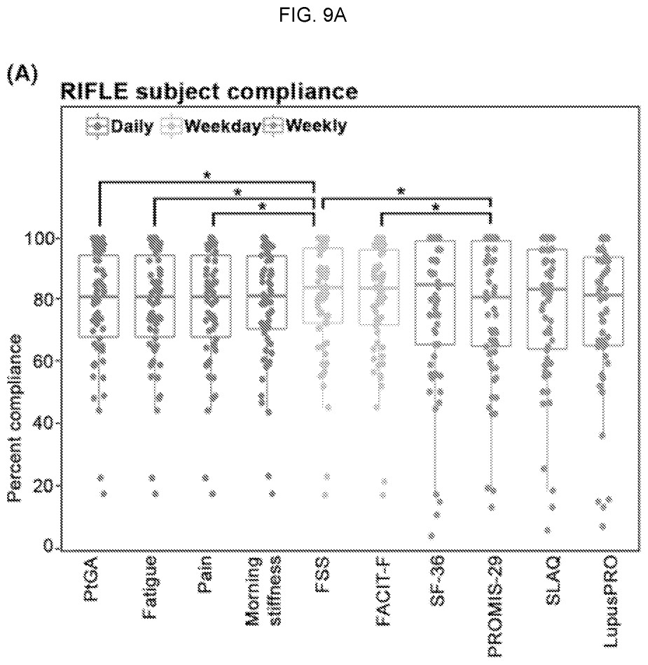

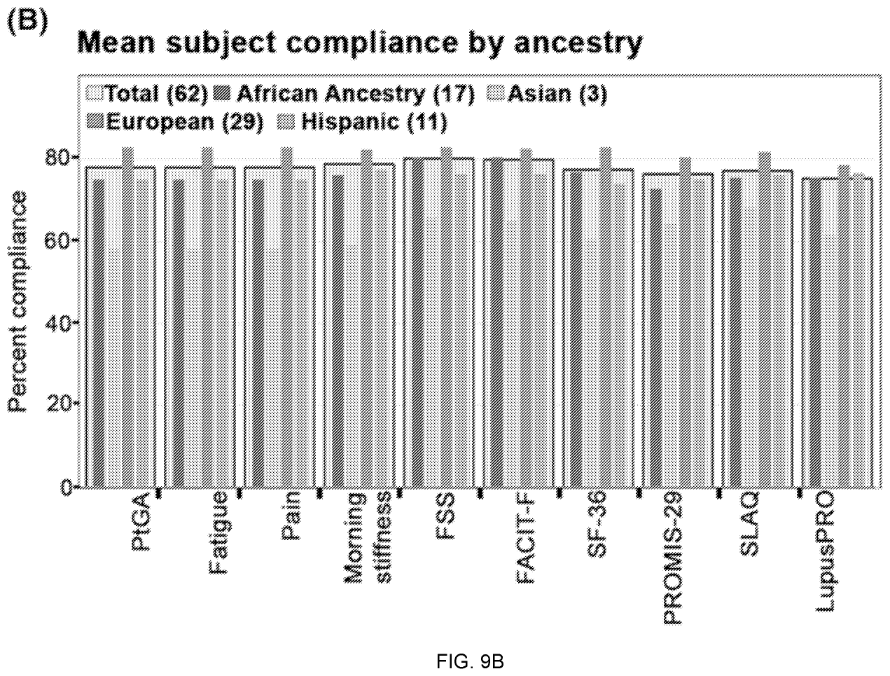

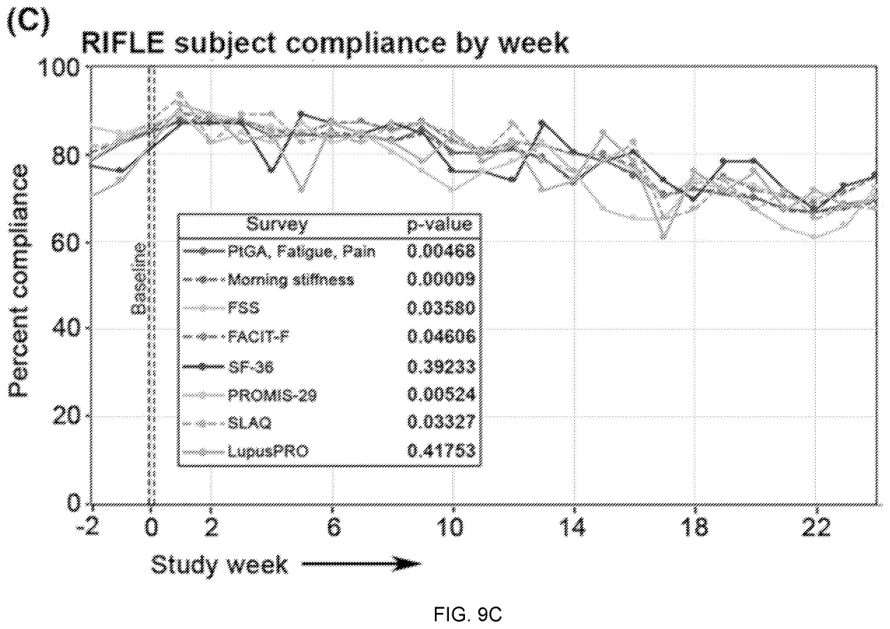

Overall and longitudinal patterns of patient compliance: Aggregate patient compliance was determined as the extent to which the ePRO requirements were fulfilled (ie, surveys were completed on time via cLuPRO). Mean compliance for mobile-app-based PRO completion was high for all surveys (more than 75.4%), with 75% of patients being at least 64.0% compliant with each measurement tool (FIG. 9A). Mean rank of compliance across all study instruments was found to be statistically significant (Friedman's ANOVA; P=0.0071) with significant differences also determined between individual instruments, such as FACIT-F and PROMIS-29 (Wilcoxon signed rank test, P<0.05). Compliance varied slightly across ancestries; however, the difference between the mean ranks across ancestries was not significant (P>0.05) (FIG. 9B). The weekday surveys yielded the highest mean compliance when they included the FSS (80.2%) and FACIT-F (80.1%). Mean subject compliance for all ePRO surveys peaked at week 1 (89.4%), declining to 71.7% by week 24 (FIG. 9C). Notably, the decline was significant for all but the two longest questionnaires (SF-36, LupusPRO). Nevertheless, mean compliance by week for all surveys remained high through trial progression (more than 60%), verifying the utility of mobile-app-based PRO reporting.

Determination of internal consistency: To evaluate the robustness of instrument consistency and survey reliability, Cronbach alpha coefficients were calculated for each multiquestion PRO. Cronbach alpha coefficient computations used all available PRO data, including the 58,173 ePROs and the 4,374 paper PROs. PRO coefficients collected via eLuPRO and the corresponding paper versions ranged from 0.73 to 0.96, suggesting that both measurement methods yielded moderate to high intersurvey reliability in measuring targeted concepts (Table 3). In addition, mobile-app-based PRO and paper PRO alpha coefficients were comparable and, in a few cases, greater than outcomes previously reported in the literature (Table 3) (22, 23, 24, 25). This was particularly noted for the SF-36, for which alpha coefficients for the mental health and social functioning domains were greater than 0.84, whereas the literature reported outcomes were 0.27 and 0.46 for social functioning and mental health, respectively (23). Furthermore, alpha coefficients between the electronic and paper administration methods were highly similar (absolute differences of less than 0.06), indicating that the within-survey question consistency is not lost with the use of the eLuPRO app.

TABLE 3
Cronbach alpha coefficients for ePROs and paper PROs

| | | Cronbach alpha | | |
|---|---|---|---|---|
| Survey | Target concept | ePRO | Paper PRO | Literature review |
| FSS | Fatigue | 0.96 | 0.96 | 0.95 (ref. 20) |
| FACIT-F | Fatigue | 0.93 | 0.88 | 0.88 (ref. 21) |
| SF-36 | Bodily Pain | 0.86 | 0.87 | 0.88 (ref. 21) |
| | General Health | 0.74 | 0.73 | 0.70 (ref. 21) |
| | Mental Health | 0.87 | 0.85 | 0.46 (ref. 21) |
| | Physical | 0.91 | 0.91 | 0.94 (ref. 21) |
| | Functioning | | | |
| | Role Emotion | 0.94 | 0.90 | 0.86 (ref. 21) |
| | Role Physical | 0.91 | 0.88 | 0.92 (ref. 21) |
| | Social | 0.89 | 0.84 | 0.27 (ref. 21) |
| | Functioning | | | |
| | Vitality | 0.79 | 0.77 | 0.70 (ref. 21) |
| PROMIS-29 | Anxiety | 0.93 | 0.93 | 0.92 (ref. 22) |
| | Depression | 0.91 | 0.92 | 0.94 (ref. 22) |
| | Fatigue | 0.91 | 0.92 | 0.95 (ref. 22) |
| | Pain Interference | 0.95 | 0.96 | 0.97(ref. 22) |
| | Physical Function | 0.91 | 0.90 | 0.92 (ref. 22) |
| | Social | 0.95 | 0.95 | 0.97 (ref. 22) |
| | Satisfaction | | | |
| SLAQ | SLE Disease | 0.88 | 0.88 | 0.87 (ref. 23) |
| | Activity | | | |
| LupusPRO | HRQOL | 0.95 | 0.95 | 0.96 (ref. 21) |
| | Non-HRQOL | 0.73 | 0.73 | 0.81 (ref. 21) |

Abbreviations: ePRO, electronic patient-reported outcome; FACIT-F, Functional Assessment of Chronic Illness Therapy-Fatigue Scale; FSS, Fatigue Severity Scale; HRQOL, health-related quality of life; PRO, patient-reported outcome; PROMIS-29, Patient Reported Outcome Measurement Information System; SF-36, Medical Outcomes Short Form 36; SLAQ, Systemic Lupus Activity Questionnaire. Breakdown of the internal consistency of all multiquestion PRO tools quantified by Cronbach alpha coefficients. Cronbach alpha coefficients range from 0 to 1, with higher values indicating greater reliability in measuring the targeted concept (column 2, “Target concept”) of every questionnaire. Alpha coefficients from this study were comparable to those of previously reported coefficients for each survey from a comprehensive literature review.

Phone-app-based patient assessment is comparable to paper administration methods: We next sought to determine whether patient assessment data collected using the eLuPRO app was similar to data collected using traditional paper methods. Pairwise Student's t tests calculated at the monthly clinic visits for every PRO survey suggested insignificant differences (P>0.05) between the app and paper-derived data in 167 of the 192 comparisons, representing 87% of all computations (FIG. 2A; five representative time points are shown). Coefficients of determination (R²) were computed at each clinic visit for every survey in order to measure the strength of the linear relationship between pairwise mobile-app-based PRO and paper PRO scores reported on the same day. Correlation coefficients ranged from 0.24 to 0.97 (FIG. 2B), and 86.5% of the 192 coefficients computed indicated a strong relationship between modes (r>0.70; R²>0.49).

To further assess agreement between survey collection methods, 192 ICCs were calculated for each survey at each clinic visit (FIG. 2C). Of the ICCs computed, 47 were indicative of moderate (0.5-0.75), 77 of good (0.75-0.9), and 64 of excellent (more than 0.90) reliability between measurement methods. All ICCs computed were significant (P<0.001) and ranged from 0.47 to 0.99 with a median ICC of 0.85. Each survey exhibited a different level of variability in ICC values amid each of the 8 site visits, of which 5 are shown. The heatmap in FIG. 2C reveals that Likert-scale PRO surveys appear more reliable between electronic and paper administration than the single-question VAS surveys (PtGA, Fatigue, Pain). The SLAQ survey emerged as the most reliable between methods, yielding ICC values above 0.9 at each site visit. FIGS. 2A-C show assessment of electronic patient-reported outcome (ePRO) versus paper PRO administration methods. FIG. 2A shows heatmap displays paired Student's/test computations for the indicated timepoints. Computations were made between either domain scores (red text for Medical Outcomes Short Form 36 [SF-36], blue text for Patient Reported Outcome Measurement Information System [PROMIS-29]), construct scores (green text for LupusPRO), or global scores (black text). Insignificant (P>0.05) and significant (P<0.05) differences between electronic and paper scores reported on the same day are indicated by color. FIG. 2B shows coefficients of determination (R²) are reported for each survey at each site visit indicated. High R² values (blue) indicate a strong linear relationship between administration methods, whereas low R² values (yellow) suggest more scatter. FIG. 2C shows intraclass correlation coefficients (ICCs) were computed to assess reliability between measurement methods. All ICCs were statistically significant (P<0.001). VAS, visual analog scale.

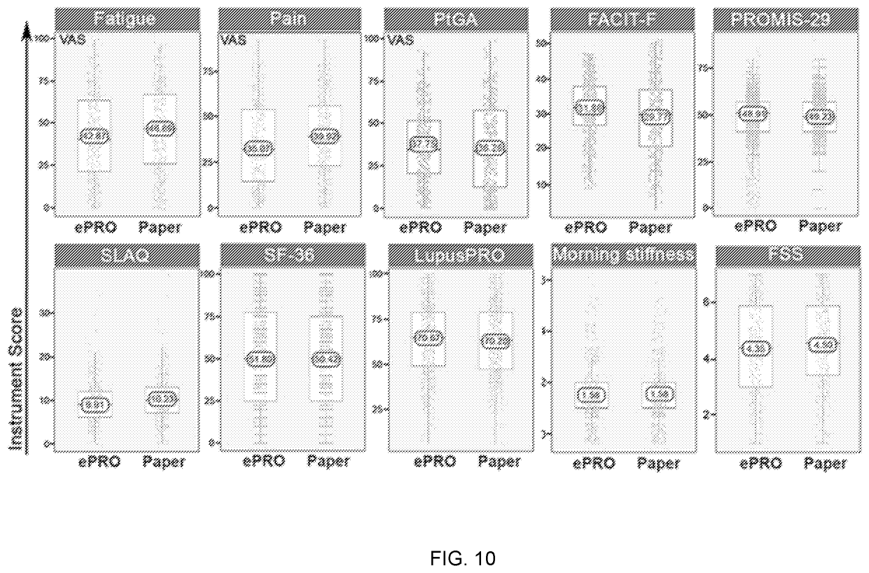

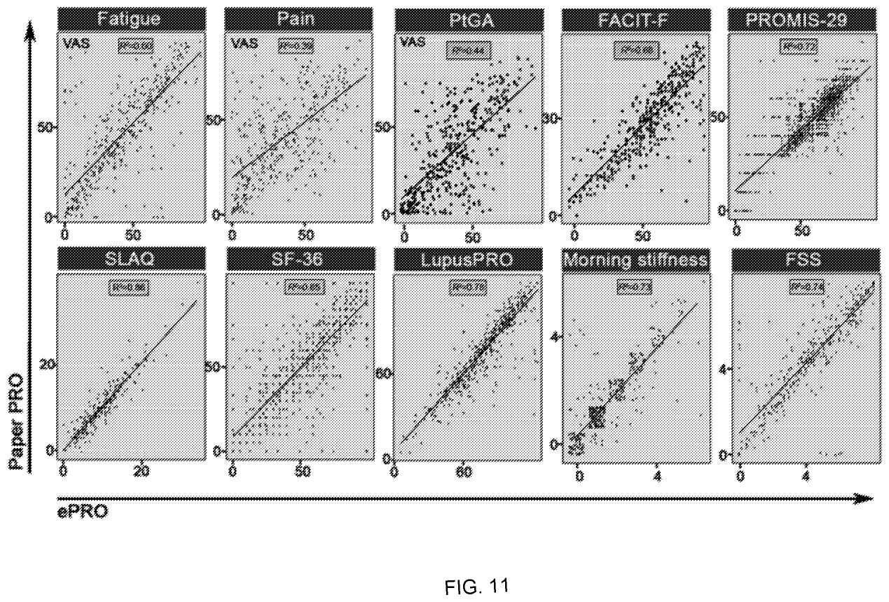

Boxplots confirm the similarity between the distribution of scores and mean responses for each administration method (FIG. 10). Additionally, scatterplots were created for each survey to visualize the correlation between PRO collection methods (FIG. 11). The SLAQ patient estimate of disease activity and LupusPRO survey displayed the strongest overall combined-visit correlation of all measurement tools with Pearson's coefficients of r=0.93 and r=0.89, respectively (FIG. 11).

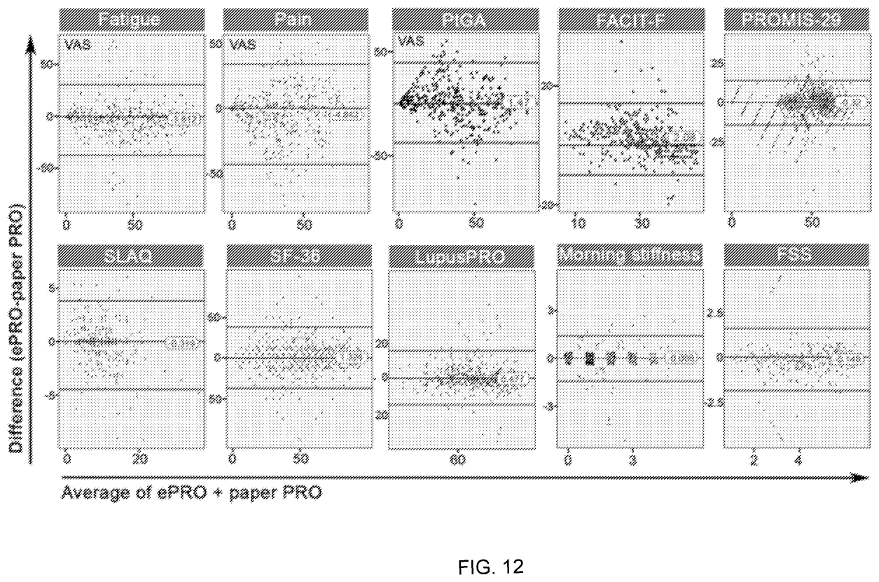

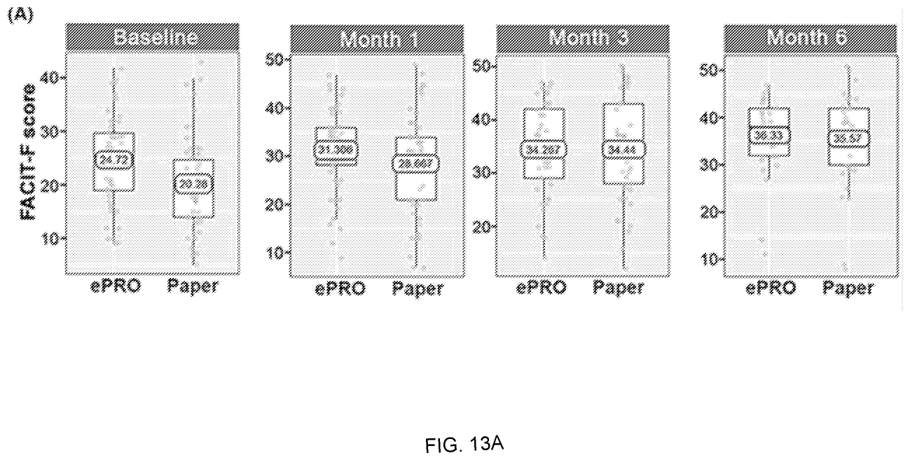

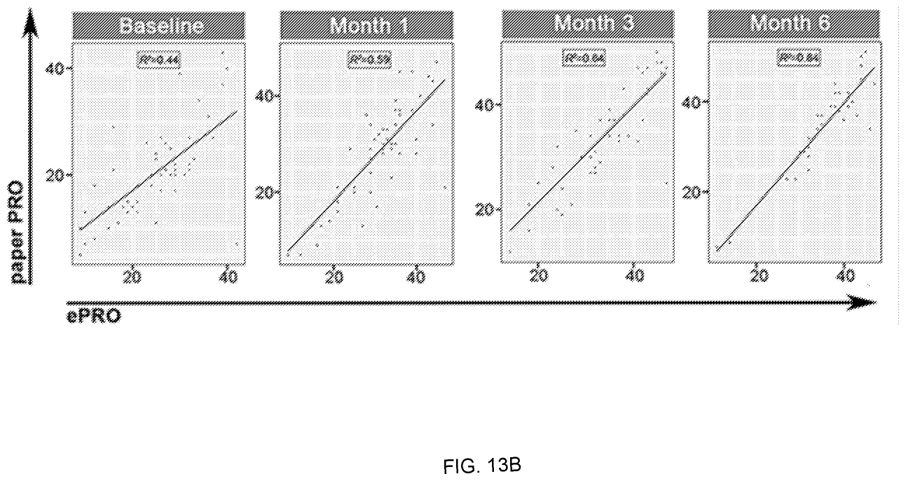

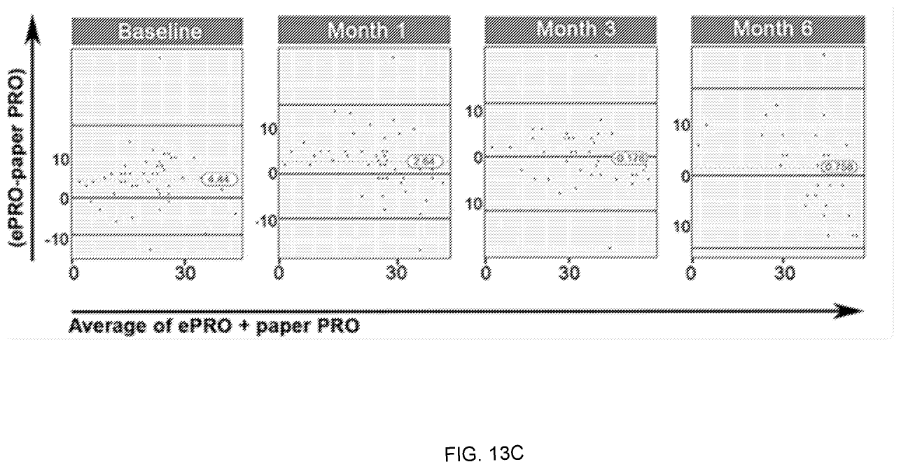

Lastly, Bland-Altman plots assessed agreement between instrument implementation (electric vs. paper) by combining all pairwise data points (FIG. 12). Average differences (biases) between measurement methods as well as confidence interval widths varied at each visit, with some visits having minor positive or negative biases for each PRO. Bland-Altman plots combining all time points revealed slight positive bias between electronic and paper methods in four PROs (PtGA, FACIT-F, SF-36, and LupusPRO; electronic scores were higher) and slight negative bias in six PROs (Fatigue, Pain, Morning Stiffness, FSS, PROMIS-29, and SLAQ; paper scores were higher). Nevertheless, the zero line was always contained within the limits of agreement in all the by-visit and combined-visit Bland-Altman plots created; therefore, there is no evidence to suggest a significant, systematic difference between administration methods. No biases surpassed the minimum clinically important difference for each PRO survey, supporting a high level of agreement between electronic and paper-reported scores. FIGS. 13A-C provides the by-visit boxplots, scatterplots, and Bland-Altman plots generated to compare the ePRO and paper PRO scores for the FACIT-F survey.

### Discussion

SLE is a clinically heterogenous autoimmune disease with a wide array of symptoms that negatively impact an individual's quality of life. The electronic capture of clinical trial source data, including PRO endpoints, is increasingly used to assess the impact of medical treatment or intervention. In general, PROs assess a range of outcomes, including symptoms, functional health and well-being, and psychological issues, to provide a holistic view of daily disease burden from the patient's perspective (3). PRO questionnaires have been used extensively in clinical trials to supplement clinical measures and provide clinicians with more information that may aid in decision-making regarding treatments. For example, changes in PRO outcomes from the RIFLE trial (www.ClinicalTrials.gov identifier NCT03098823) showed that treating rheumatoid arthritis patients with upadacitinib can lead to clinically significant relief from symptoms (26).

Mobile-app-based PRO data collection in clinical trials offers many advantages over traditional paper-based methods: they are not location dependent, they can be conducted in an unsupervised manner, and, most importantly, they allow for accurate and real-time reporting of symptoms. Many other SLE-specific patient-centered apps, such as LupusTracker PRO (ToTheHand, LLC) and My Lupus Log (GlaxoSmithKline), have been developed in order to empower patients in the daily management of their disease and/or in order to reduce the communication gap between SLE patients and their providers (27). In addition, ePRO apps have been developed for other inflammatory diseases, including rheumatoid arthritis. Whereas these and other studies demonstrate app compliance, few, if any, provide validation analyses (ie, the app successfully measures the domain of interest). Here, the eLuPRO phone-based app was developed for a phase 4 clinical trial in order to examine real-time changes in multiple different PRO instruments during a period of therapeutic intervention. In addition to evaluation by PROs, the inclusion of SLAQ for the personal assessment of disease activity within the eLuPRO framework provides an additional tool for patients to judge the benefit of care received. It should be noted that RIFLE was biased toward subjects experiencing increased fatigue (FACIT-F score of more than 25), which may decrease its application to the greater lupus population. Nonetheless, our results indicate that eLuPRO was both functional and widely used by patients throughout the trial, with several subjects continuing to use the eLuPRO tools beyond their enrollment in the trial. Our double baseline approach was useful in that it provided a period of app training and it allowed us to collect multiple data points before initiation of the intervention.

Patient demographics reveal a diverse range of ancestral backgrounds, with over half of enrolled subjects of non-European descent. This is important given that certain ancestral groups experience the disease more severely, such as those of African ancestry, who account for 43% of all SLE subjects yet typically represent a low proportion of trial participants (less than 14%) (28, 29, 30). Overall compliance with app usage was high (more than 75%) for most surveys across demographics, particularly the weekly FSS and FACIT-F surveys with 80% mean compliance, demonstrating the utility of electronic patient-directed data collection.

In order to validate the extensive PRO information collected via eLuPRO, we sought to verify the equivalence of paper and electronic administration methods for all surveys. There was remarkable comparability with a significant difference in only 13% of comparisons using a Student's t test to examine differences in mean scores between methods. Notably, the pain intensity domain of the PROMIS-29 instrument was the only survey that resulted in a consistently significant difference in method score at the in-clinic visits yet ICCs and R² values indicated excellent agreement. One reason for the discrepancy in results is that the t test does not consider patient bias (differences at the level of individual patient), but rather it compares the mean score for each administration method at each time point. Additionally, multiquestion surveys in which one score is reported showed greater correlation between paper and ePRO responses than that of single-question instruments. ICC analysis further revealed that reliability between administration methods was acceptable, and oftentimes high, for all PROs at every in-clinic visit. Administration agreement appears slightly lower in the VAS questions than in the Likert-scaled surveys. Compared with instruments using a Likert scale to obtain ordinal-level measurements, instruments using the 100-mm VAS scale allow for the collection of measurements with more variability. Although this produces more fine-grained responses based on a line continuum, data obtained using VAS are generally more variable because of the “unstructured” nature of the scale; it is therefore not surprising that the VAS questions performed less well. Nonetheless, the ICC values for the VAS PROs show an increasing trend over the course of the trial, suggesting that agreeability may improve as more surveys are taken. All Bland-Altman plots showed points that were roughly scattered evenly around the zero line, suggesting no consistent bias between paper-based and mobile-app-based PRO scores.

Relatively small size of the study, made up for by the large number of PROs collected. In addition, the study inadvertently collected data from patients with high medical literacy in a structured academic setting; it is, therefore, uncertain whether the app will work comparably in general practice with patients of varying health literacy profiles (31). Although construct validity had not been demonstrated at the time this study was carried out, the acceptability of apps can now be assessed with rating scales, such as the Mobile Application Rating Scale (MARS) (32), that could be useful to evaluate the app more fully. However, patient feedback indicated frustration with the redundant nature of the selected PRO instruments, manifesting as “app fatigue” and likely contributing to declining compliance over time. Although there was some manifestation of app fatigue in this trial, in the future this might be mitigated by rewards, simplification, or providing patient access to their personal data. Despite these caveats, this study represents the first successful attempt to validate a wide range of PRO information from lupus patients with a mobile app. We found that collecting data via phone app is both feasible and valid and is likely to detect changes related to treatment and/or spontaneous fluctuations in disease. The collection of dense PRO data permits analysis of real trends rather than intermittent pools of information, allows for assessment in a patient's regular environment, and is resistant to obstructions in data quality and blank entries. Importantly, the use of the eLuPRO app permits real-time decision-making because data collection and entry into the database are automatically reported daily. The data suggest that PRO collection by app could replace that done in the clinic by paper or electronic methodology. Future analyses will expand on these current observations and will focus on identifying those health domains that best correlate with clinical changes in disease activity, reducing both redundancy and response burden.

References (All publications, patents, and patent applications mentioned in this specification are herein incorporated by reference to the same extent as if each individual publication, patent, or patent application was specifically and individually indicated to be incorporated by reference)
1. Kiriakidou M, Ching C. Systemic lupus erythematosus. Ann Intern Med 2020; 172:ITC81-96.
2. Gensous N, Marti A, Barnetche T, Blanco P, Lazaro E, Seneschal J, et al, Predictive biological markers of systemic lupus erythematosus flares: a systematic literature review. Arthritis Res Ther 2017; 19:238.
3. Schmeding A, Schneider M Fatigue, health-related quality of life and other patient-reported outcomes in systemic lupus erythematosus. Best Pract Res Clin Rheumatol 2013,27:363-75
4. Yen J C, Neville C, Fortin P R. Discordance between patients and their physicians in the assessment of lupus disease activity: relevance for clinical trials. 1999;8:660-70.
5. Romero-Diaz J, Isenberg D, Ramsey-Goldman R. Measures of adult systemic lupus erythematosus: updated version of British Isles Lupus Assessment Group (BILAG 2004), European Consensus Lupus Activity Measurements (ECLAM), Systemic Lupus Activity Measure, Revised (SLAM-R), Systemic Lupus Activity Questionnaire for Population Studies (SLAQ), Systemic Lupus Erythematosus Disease Activity Index 2000 (SLEDAI-2K), and Systemic Lupus International Collaborating Clinics/American College of Rheumatology Damage Index (SDI). Arthritis Care Res (Hoboken) 2011;63.
6. Azizoddin D R, Jolly M, Arora S, Yelin E, Katz P. Patient-reported outcomes predict mortality in lupus. Arthritis Care Res (Hoboken) 2019;71:1028-35.
7. Jolly M, Pickard A S, Block J A, Kumar R B, Mikolaitis R A, Wilke C T, et al. Disease-specific patient reported outcome tools for systemic lupus erythematosus. Semin Arthritis Rheum 2012;42:56-65.
8 Rolfson O, Salomonsson R, Dahlberg L E, Garellick G. Internet-based follow-up questionnaire for measuring patient-reported outcome after total hip replacement surgery-reliability and response rate. Value in Health 2011; 14:316-21.
9. Muehlhausen W, Doll H, Quadri N, Fordham B, O'Donohoe P, Dogar N, et al. Equivalence of electronic and paper administration of patient-reported outcome measures: a systematic review and meta-analysis of studies conducted between 2007 and 2013. Health Qual Life Outcomes 2015:13:167.
10. Bingham C O 3rd, Gaich C L, DeLozier A M, Engstrom K D, Naegeli A N, de Bono S, et al. Use of daily electronic patient-reported outcome (PRO) diaries in randomized controlled trials for rheumatoid arthritis: rationale and implementation. Trials 2019;22;20:182 [erratum in: Trials 2019;20:322].
11. Richter J G, Nannen C, Chehab G, Acar H, Becker A, Willers R, et al. Mobile app-based documentation of patient-reported outcomes—3-months results from a proof-of-concept study on modern rheumatology patient management. Arthritis Res Ther 2021; 19;23:121.
12. Thanou A, James J, Arriens C, Aberle T, Chakravarty E, Rawdon J, et al. Scoring systemic lupus erythematosus (SLE) disease activity with simple, rapid outcome measures. Lupus Sci Med 2019;6: e000365.
13. Alten R, Holt R, Grahn A, Rice P, Kent J, Buttgereit F, et al. Morning stiffness response with delayed-release prednisone after ineffective course of immediate-release prednisone. Scand J Rheumatol 2015;44:354-58.
14. Ad Hoc Committee on Systemic Lupus Erythematosus Response Criteria for Fatigue. Measurement of fatigue in systemic lupus erythematosus: a systemic review. Arthritis Rheum 2007:57:1348-57.
15. Kosinski M, Gajria K, Fernandes A W, Cella D. Qualitative validation of the FACIT-fatigue scale in systemic lupus erythematosus. Lupus 2013;22:422-30.
16. Stoll T, Gordon C, Seifert B, Richardson K, Malik J, Bacon P A, et al. Consistency and validity of patient administered assessment of quality of life by the MOS SF-36; its association with disease activity and damage in patients with systemic lupus erythematosus. J Rheumatol 1997;24: 1608-14.
17. Katz P, Pedro S, Michaud K. Performance of the patient-reported outcomes measurement information system 29-item profile in rheumatoid arthritis, osteoarthritis, fibromyalgia, and systemic lupus erythematosus. Arthritis Care Res (Hoboken) 2017;69:1312-21.
18. Chew L D, Bradley K A, Boyko E J. Brief questions to identify patients with inadequate health literacy Fam Med 2004;36:588-94.
19. Katz P, Dall'Era M, Trupin L, Rush S, Murphy L B, Lanata C, et al. Impact of limited health literacy on patient-reported outcomes in systemic lupus erythematosus. Arthritis Care Res (Hoboken) 2021;73:110-19.
20. Mcgraw K O, Wong S P. Forming inferences about some intraclass correlation coefficients. American Psychological Association 1996; 1:30-46.
21. Giavarina D. Understanding Bland Altman analysis. Biochem Med (Zagreb) 2015;25:141-51.
22. Rosti-Otajärvi E, Hämäläinen P, Wiksten A, Hakkarainen T, Ruutiainen J. Validity and reliability of the Fatigue Severity Scale in Finnish multiple sclerosis patients. Brain Behav 2017; 7: e00743.
23. Azizoddin D R, Weinberg S, Gandhi N, Arora S, Block J A, Sequeira W, et al. Validation of the LupusPRO version 1.8: an update to a disease-specific patient-reported outcome tool for systemic lupus erythematosus. Lupus 2018:27:728-37.
24. Schnall R, Liu J, Cho H, Hirshfield S, Siegel K, Olender S. A health-related quality-of-life measure for use in patients with HIV: a validation study. AIDS Patient Care STDs 2017;31:43-8.
25. Yazdany J, Yelin E H, Panopalis P, Trupin L, Julian L, Katz P P. Validation of the Systemic Lupus Erythematosus Activity Questionnaire in a large observational cohort. Arthritis Rheum 2008; 59:136-43.
26. Strand V. Schiff M, Tundia N, Friedman A, Meerwein S, Pangan A, et al. Effects of upadacitinib on patient-reported outcomes: results from SELECT-BEYOND, a phase 3 randomized trial in patients with rheumatoid arthritis and inadequate responses to biologic disease-modifying antirheumatic drugs. Arthritis Res Ther 2019;21:263.
27. Dantas L O, Weber S, Osani M C, Bannuru R R, McAlindon T E, Kasturi S. Mobile health technologies for the management of systemic lupus erythematosus a systematic review. Lupus 2020:29:144-56.
28. Falasinnu T, Chaichian Y, Bass M B, Simard J F. The Representation of gender and race/ethnic groups in randomized clinical trials of individuals with systemic lupus erythematosus. Curr Rheumatol Rep 2018;20:1-11.

29. Williams EM, Bruner L, Adkins A, Vrana C, Logan A, Kamen D, et al. I too, am America: a review of research on systemic lupus erythematosus in African-Americans. Lupus Sci Med 2016;3: e000144
30. Anjorin A, Lipsky P. Engaging African ancestry participants in SLE clinical trials. Lupus Sci 2018;5.

31. Bakker MM, Putrik P, Rademakers J, van de Laar M, Vonkeman H, Kok M R, et al. Addressing health literacy needs in rheumatology-which patient health literacy profiles need the attention of health professionals? Arthritis Care Res (Hoboken) 2020; 73:100-9.
32. Terhorst Y, Philippi P. Sander L, Schultchen D, Paganini S, Bardus M, et al. Validation of the mobile application rating scale. Plos One 2020; 15: e0241480

### Example 2: Unsupervised Clustering of Lupus Patient-Reported Outcome Data Identifies Patient Groups with Differences in SLEDAI and Physician Global Assessment

Systemic lupus erythematosus (SLE) is an autoimmune disease with heterogeneous clinical presentations. Patient-reported outcomes (PROs) can aid in the measurement of the burden of disease. However, PRO information often does not correlate with physician-evaluated disease activity. This study employed unsupervised clustering analysis of PRO information from multiple instruments to identify subsets of patients with different levels of physician-evaluated disease activity.

### Methods

The electronic PRO (ePRO) data of patients meeting the American College of Rheumatology (ACR) definition of SLE were collected over a period of 6 months as part of a multicenter clinical trial (NCT03098823). A smartphone application was developed to collect 10 separate PROs according to a schedule, collecting over 70,000 total records. A dataset containing the mean values of the PROs for each patient across the whole study was prepared. After preprocessing, unsupervised clustering analysis of the whole-study PRO means data was carried out. Three metrics of clustering performance were used to assess the best model and optimal number of clusters between Gaussian mixture modeling with variational inference, k-means clustering and hierarchical clustering. This analysis was repeated on a dataset of monthly PRO means where each mean was calculated from PROs recorded in the month preceding a clinic visit. Classification models were trained to distinguish cluster assignment in the monthly means dataset using a smaller subset of the PROs used as features for clustering. Model performance was assessed by area under the receiver operating characteristic curve (AUC).

### Results

Clustering analysis of PRO information identified two groups from a diverse sample of 62 total SLE patients that differed in their symptoms as assessed by ePRO questionnaires. Cluster 1 contained 51 patients, whereas cluster 2 contained 11 patients; in general, cluster 1 patients reported significantly milder self-reported symptoms (Table 1). Notably, cluster 1 and 2 patients manifested significantly different SLEDAI as well as Physician Global Assessment scores (FIG. 6B). These results were largely repeated in the clustering analysis of patients' monthly PRO means, with a large, healthier cluster (n=338) and a smaller cluster (n=70) with more intense disease activity (FIGS. 6C and D). A Support Vector Machine (SVM) classification model was able to classify patients into Group 1 or 2 reliably using only four PROs as input features, including measures of social functioning, Patient Global Assessment, pain intensity, and mental health. The model trained on these four features had a test set AUC of 0.98 (FIG. 6G).

### Conclusion

Two groups of SLE patients that differ significantly in physician measures of SLE activity were described by an unsupervised clustering analysis of ePROs collected using a novel smartphone application. Collection of ePRO data and patient cluster assignment based on those data could assist health care providers in monitoring SLE patients and personalizing their treatment.

What is claimed is:

1. A method for evaluation and/or treatment of a disease in a patient, the method comprising:
receiving, by at least one processor of a first device, patient-reported outcome data from a second device, the patient-reported outcome data indicative of at least one of a patient's reactions to patient's health, or a first method of evaluation or treatment using a pharmaceutical or biological treatment;
validating, by the at least one processor, the patient-reported outcome data;
inputting, by the at least one processor, the patient-reported outcome data to a first machine learning model;
generating, using the first machine learning model, based on the patient-reported outcome data, i) a score indicative of at least one of an activity of the patient's disease, an effectiveness of current treatment, or severity of the patient's reactions, and/or ii) an inference indicative of the disease state of the patient; and
generating, recommending, and/or selecting based on the score and/or the inference, an action item and/or a second method of evaluation or treatment for the patient, optionally using a second machine learning model.

2. The method of claim 1, further comprising:
training the first machine learning model to generate the score based on a percentage of scores associated with adjusting the first method of evaluation or treatment.

3. The method of claim 1, generating, recommending, and/or selecting the action item and/or the second method of evaluation or treatment for the patient is further based on a comparison of the score to a score threshold.

4. The method of claim 1, wherein the second method of evaluation or treatment is different than the first method of evaluation or treatment.

5. The method of claim 1, wherein the second method of evaluation or treatment is the same as the first method of evaluation or treatment.

6. The method of claim 1, further comprising:
receiving at least one of biometric data or device motion data,
wherein generating the score and/or inference is further based on the at least one of the biometric data or the device motion data.

7. The method of claim 6, wherein the biometric data comprises at least one of sleep data, breathing data, body temperature data, or heart rate data.

8. The method of claim 6, wherein the device motion data comprises accelerometer data indicative of activity of the patient.

9. The method of claim 1, wherein the inference is, whether the PRO data is indicative of the patient having active disease, or not having active disease.

10. The method of claim 1, wherein the action item comprises scheduling an appointment, visit, and/or consultation with a healthcare professional.

11. The method of claim 1, further comprising performing the action item, performing the second method of evaluation, and/or administering the second treatment to the patient.

12. A method for predicting a Physician's Global Assessment Score, the method comprising:
receiving, by at least one processor of a first device, patient-reported outcome data from a second device, the patient reported outcome data indicative of a patient's disease status or reactions of the patient to a first method of evaluation or treatment using a pharmaceutical or biological treatment;
validating, by the at least one processor, the patient-reported outcome data;
receiving, by the at least one processor, documents associated with visits to a doctor;
inputting, by the at least one processor, the patient-reported outcome data and the documents to a first machine learning model;
generating, using the first machine learning model, based on the patient-reported outcome data and the documents, a prediction of a Physician's Global Assessment Score indicative of a patient's status or a severity of the patient's reactions; and
generating, recommending, and/or selecting based on the prediction of the Physician's Global Assessment Score, an action item, and/or a second method of evaluation or treatment for the patient, optionally using a second machine learning model.

13. The method of claim 12, further comprising:
training the first machine learning model to generate the Physician's Global Assessment Score based on a percentage of scores associated with adjusting the first method of evaluation or treatment.

14. The method of claim 12, wherein generating the second method of evaluation or treatment for the patient is further based on a comparison of the Physician's Global Assessment Score to a score threshold.

15. The method of claim 12, wherein the second method of evaluation or treatment is different than the first method of evaluation or treatment.

16. The method of claim 12, wherein the second method of evaluation or treatment is the same as the first method of evaluation or treatment.

17. The method of claim 12, further comprising:
receiving at least one of biometric data or device motion data,
wherein generating the Physician's Global Assessment Score is further based on the at least one of the biometric data or the device motion data.

18. A method for diagnosis and/or treatment of lupus in a patient, the method comprising:
receiving, by at least one processor of a first device, patient-reported outcome data from a second device, the patient-reported outcome data indicative of a patient's reactions to i) patient's health, ii) to a first method of evaluation and/or ii) to a first method of treatment for Lupus using a pharmaceutical or biological treatment;
validating, by the at least one processor, the patient-reported outcome data;
optionally receiving, by the at least one processor, a doctor's data comprising data from the patient's visits to a doctor;
inputting, by the at least one processor, the patient-reported outcome data and optionally the doctor's data to a first machine learning model;
generating, using the first machine learning model, based on the patient-reported outcome data and optionally the doctor's data, i) a score indicative of activity of a patient's disease and the patient's reactions, and/or ii) an inference indicative of the lupus disease state of the patient; and
adjusting, generating, recommending, and/or selecting, based on the score and/or inference, an action item and/or a second method of evaluation or treatment for the patient, optionally using a second machine learning model.

19. The method of claim 18, further comprising:
training the first machine learning model to generate the SLEDAI score based on a percentage of scores associated with adjusting the first method of evaluation or treatment.

20. The method of claim 18, wherein generating the second method of evaluation or treatment for the patient is further based on a comparison of the score SLEDAI to a score threshold.

21. The method of claim 18, wherein the second method of evaluation or treatment is different than the first method of evaluation or treatment.

22. The method of claim 18, wherein the second method of evaluation or treatment is the same as the first method of evaluation or treatment.

23. The method of claim 18, further comprising:
receiving at least one of biometric data or device motion data,
wherein generating the SLEDAI score and/or the inference is further based on the at least one of the biometric data or the device motion data.

24. The method of claim 18, wherein the inference is whether the PRO data from the patient is indicative of the patient having active lupus, or not having active lupus.

25. The method of claim 18, wherein the PRO data comprises one or more of SLAQ data, HRQOL data, Non-HRQOL data, Fatigue VAS data, Pain VAS data, PtGA data, FSS data, FACIT-F data, Morning Stiffness data, Fatigue data, Sleep disturbance data, Depression data, Anxiety data, Pain Intensity data, Pain interference data, Satisfaction with social role data, physical function data, vitality data, bodily pain data, general health data, mental health data, physical function data, role emotional data, role physical data and social function data.

26. The method of claim 18, wherein the PRO data comprises PtGA data, Pain Intensity data, mental health data, and social function data.

27. The method of claim 18, wherein the first machine learning model has a receiver operating characteristic (ROC) curve with an Area-Under-Curve (AUC) of at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

28. The method of claim 18, wherein the first machine learning model generate the score and/or the inference using linear regression, logistic regression (LOG), Ridge regression, Lasso regression, an elastic net (EN) regression, support vector machine (SVM), gradient boosted machine (GBM), k nearest neighbors (kNN), generalized linear model (GLM), naïve Bayes (NB) classifier, neural network, Random Forest (RF), deep learning algorithm, linear discriminant analysis (LDA), decision tree learning (DTREE), adaptive boosting (ADB), Classification and Regression Tree (CART), hierarchical clustering, or any combination thereof.

29. The method of claim 18, further comprising performing the action item, performing the second method of evaluation, and/or administering the second treatment to the patient.

30. The method of claim 18, wherein the second treatment is configured to treat active lupus.

31. The method of claim 18, wherein the second treatment is configured to treat reduce severity of active lupus.

32. The method of claim 18, wherein the second treatment is configured to reduce risk of having active lupus.

33. The method of claim 18, wherein the patient has lupus.
